SEQUENCE LISTING (14 sequences)

SEQ ID NO: 1 - DNA, 2957 bp, Homo sapiens
gccagtcaccttcagtttctggagctggccgtcaacatgtcctttcctaaggcgcccttgaaacgattcaatgacccttctggttgtgcaccatctccaggtgcttatgatgttaaaactttagaagtattgaaaggaccagtatcctttcagaaatcacaaagatttaaacaacaaaaagaatctaaacaaaatcttaatgttgacaaagatactaccttgcctgcttcagctagaaaagttaagtcttcggaatcaaagattcgtgttcttctacaggaacgtggtgcccaggacagccggatccaggatctggaaactgagttggaaaagatggaagcaaggctaaatgctgcactaagggaaaaaacatctctctctgcaaataatgctacactggaaaaacaacttattgaattgaccaggactaatgaactactaaaatctaagttttctgaaaatggtaaccagaagaatttgagaattctaagcttggagttgatgaaacttagaaacaaaagagaaacaaagatgaggggtatgatggctaagcaagaaggcatggagatgaagctgcaggtcacccaaaggagtctcgaagagtctcaagggaaaatagcccaactggagggaaaacttgtttcaatagagaaagaaaagattgatgaaaaatctgaaacagaaaaactcttggaatacatcgaagaaattagttgtgcttcagatcaagtggaaaaatacaagctagatattgcccagttagaagaaaatttgaaagagaagaatgatgaaattttaagccttaagcagtctcttgaggagaatattgttatattatctaaacaagtagaagatctaaatgtgaaatgtcagctgcttgaaaaagaaaaagaagaccatgtcaacaggaatagagaacacaacgaaaatctaaatgcagagatgcaaaacttaaaacagaagtttattcttgaacaacaggaacgtgaaaagcttcaacaaaaagaattacaaattgattcacttctgcaacaagagaaagaattatcttcgagtcttcatcagaagctctgttcttttcaagaggaaatggttaaagagaagaatctgtttgaggaagaattaaagcaaacactggatgagcttgataaattacagcaaaaggaggaacaagctgaaaggctggtcaagcaattggaagaggaagcaaaatctagagctgaagaattaaaactcctagaagaaaagctgaaagggaaggaggctgaactggagaaaagtagtgctgctcatacccaggccaccctgcttttgcaggaaaagtatgacagtatggtgcaaagccttgaagatgttactgctcaatttgaaagctataaagcgttaacagccagtgagatagaagatcttaagctggagaactcatcattacaggaaaaagcggccaaggctgggaaaaatgcagaggatgttcagcatcagattttggcaactgagagctcaaatcaagaatatgtaaggatgcttctagatctgcagaccaagtcagcactaaaggaaacagaaattaaagaaatcacagtttcttttcttcaaaaaataactgatttgcagaaccaactcaagcaacaggaggaagactttagaaaacagctggaagatgaagaaggaagaaaagctgaaaaagaaaatacaacagcagaattaactgaagaaattaacaagtggcgtctcctctatgaagaactatataataaaacaaaaccttttcagctacaactagatgcttttgaagtagaaaaacaggcattgttgaatgaacatggtgcagctcaggaacagctaaataaaataagagattcatatgctaaattattgggtcatcagaatttgaaacaaaaaatcaagcatgttgtgaagttgaaagatgaaaatagccaactcaaatcggaagtatcaaaactccgctgtcagcttgctaaaaaaaaacaaagtgagacaaaacttcaagaggaattgaataaagttctaggtatcaaacactttgatccttcaaaggcttttcatcatgaaagtaaagaaaattttgccctgaagaccccattaaaagaaggcaatacaaactgttaccgagctcctatggagtgtcaagaatcatggaagtaaacatctgagaaacctgttgaagattatttcattcgtcttgttgttattgatgttgctgttattatatttgacatgggtattttataatgttgtatttaattttaactgccaatccttaaatatgtgaaaggaacattttttaccaaagtgtcttttgacattttattttttcttgcaaatacctcctccctaatgctcacctttatcacctcattctgaaccctttcgctggctttccagcttagaatgcatctcatcaacttaaaagtcagtatcatattattatcctcctgttctgaaaccttagtttcaagagtctaaaccccagattcttcagcttgatcctggaggtcttttctagtctgagcttctttagctaggctaaaacaccttggcttgttattgcctctactttgattctgataatgctcacttggtcctacctattatccttctacttgtccagttcaaataagaaataaggacaagcctaacttcatagaaacctctctatttttaatcagttgtttaataatttacaggttcttaggctccatcctgtttgtatgaaattataatctgtggattggcctttaagcctgcattcttaacaaactcttcagttaattcttagatacactaaaaatctgagaaactctacatgtaactatttcttcagagtttgtcatatactgcttgtcatctgcatgtctactcagcatttgattaacatttgtgtaatatgaaataaaattacacagtaagtcatttaaccaaaaaaaaaaaaaaaa

SEQ ID NO: 2 - protein, 709 aa, Homo sapiens
MSFPKAPLKRFNDPSGCAPSPGAYDVKTLEVLKGPVSFQKSQRFKQQKESKQNLNVDKDTTLPASARKVKSSESKIRVLLQERGAQDSRIQDLETELEKMEARLNAALREKTSLSANNATLEKQLIELTRTNELLKSKFSENGNQKNLRILSLELMKLRNKRETKMRGMMAKQEGMEMKLQVTQRSLEESQGKIAQLEGKLVSIEKEKIDEKSETEKLLEYIEEISCASDQVEKYKLDIAQLEENLKEKNDEILSLKQSLEENIVILSKQVEDLNVKCQLLEKEKEDHVNRNREHNENLNAEMQNLKQKFILEQQEREKLQQKELQIDSLLQQEKELSSSLHQKLCSFQEEMVKEKNLFEEELKQTLDELDKLQQKEEQAERLVKQLEEEAKSRAEELKLLEEKLKGKEAELEKSSAAHTQATLLLQEKYDSMVQSLEDVTAQFESYKALTASEIEDLKLENSSLQEKAAKAGKNAEDVQHQILATESSNQEYVRMLLDLQTKSALKETEIKEITVSFLQKITDLQNQLKQQEEDFRKQLEDEEGRKAEKENTTAELTEEINKWRLLYEELYNKTKPFQLQLDAFEVEKQALLNEHGAAQEQLNKIRDSYAKLLGHQNLKQKIKHVVKLKDENSQLKSEVSKLRCQLAKKKQSETKLQEELNKVLGIKHFDPSKAFHHESKENFALKTPLKEGNTNCYRAPMECQESWK

SEQ ID NO: 3 - DNA, 3002 bp, Homo sapiens
gccagtcaccttcagtttctggagctggccgtcaacatgtcctttcctaaggcgcccttgaaacgattcaatgacccttctggttgtgcaccatctccaggtgcttatgatgttaaaactttagaagtattgaaaggaccagtatcctttcagaaatcacaaagatttaaacaacaaaaagaatctaaacaaaatcttaatgttgacaaagatactaccttgcctgcttcagctagaaaagttaagtcttcggaatcaaaggaatctcaaaagaatgataaagatttgaagatattagagaaagagattcgtgttcttctacaggaacgtggtgcccaggacagccggatccaggatctggaaactgagttggaaaagatggaagcaaggctaaatgctgcactaagggaaaaaacatctctctctgcaaataatgctacactggaaaaacaacttattgaattgaccaggactaatgaactactaaaatctaagttttctgaaaatggtaaccagaagaatttgagaattctaagcttggagttgatgaaacttagaaacaaaagagaaacaaagatgaggggtatgatggctaagcaagaaggcatggagatgaagctgcaggtcacccaaaggagtctcgaagagtctcaagggaaaatagcccaactggagggaaaacttgtttcaatagagaaagaaaagattgatgaaaaatctgaaacagaaaaactcttggaatacatcgaagaaattagttgtgcttcagatcaagtggaaaaatacaagctagatattgcccagttagaagaaaatttgaaagagaagaatgatgaaattttaagccttaagcagtctcttgaggagaatattgttatattatctaaacaagtagaagatctaaatgtgaaatgtcagctgcttgaaaaagaaaaagaagaccatgtcaacaggaatagagaacacaacgaaaatctaaatgcagagatgcaaaacttaaaacagaagtttattcttgaacaacaggaacgtgaaaagcttcaacaaaaagaattacaaattgattcacttctgcaacaagagaaagaattatcttcgagtcttcatcagaagctctgttcttttcaagaggaaatggttaaagagaagaatctgtttgaggaagaattaaagcaaacactggatgagcttgataaattacagcaaaaggaggaacaagctgaaaggctggtcaagcaattggaagaggaagcaaaatctagagctgaagaattaaaactcctagaagaaaagctgaaagggaaggaggctgaactggagaaaagtagtgctgctcatacccaggccaccctgcttttgcaggaaaagtatgacagtatggtgcaaagccttgaagatgttactgctcaatttgaaagctataaagcgttaacagccagtgagatagaagatcttaagctggagaactcatcattacaggaaaaagcggccaaggctgggaaaaatgcagaggatgttcagcatcagattttggcaactgagagctcaaatcaagaatatgtaaggatgcttctagatctgcagaccaagtcagcactaaaggaaacagaaattaaagaaatcacagtttcttttcttcaaaaaataactgatttgcagaaccaactcaagcaacaggaggaagactttagaaaacagctggaagatgaagaaggaagaaaagctgaaaaagaaaatacaacagcagaattaactgaagaaattaacaagtggcgtctcctctatgaagaactatataataaaacaaaaccttttcagctacaactagatgcttttgaagtagaaaaacaggcattgttgaatgaacatggtgcagctcaggaacagctaaataaaataagagattcatatgctaaattattgggtcatcagaatttgaaacaaaaaatcaagcatgttgtgaagttgaaagatgaaaatagccaactcaaatcggaagtatcaaaactccgctgtcagcttgctaaaaaaaaacaaagtgagacaaaacttcaagaggaattgaataaagttctaggtatcaaacactttgatccttcaaaggcttttcatcatgaaagtaaagaaaattttgccctgaagaccccattaaaagaaggcaatacaaactgttaccgagctcctatggagtgtcaagaatcatggaagtaaacatctgagaaacctgttgaagattatttcattcgtcttgttgttattgatgttgctgttattatatttgacatgggtattttataatgttgtatttaattttaactgccaatccttaaatatgtgaaaggaacattttttaccaaagtgtcttttgacattttattttttcttgcaaatacctcctccctaatgctcacctttatcacctcattctgaaccctttcgctggctttccagcttagaatgcatctcatcaacttaaaagtcagtatcatattattatcctcctgttctgaaaccttagtttcaagagtctaaaccccagattcttcagcttgatcctggaggtcttttctagtctgagcttctttagctaggctaaaacaccttggcttgttattgcctctactttgattctgataatgctcacttggtcctacctattatccttctacttgtccagttcaaataagaaataaggacaagcctaacttcatagaaacctctctatttttaatcagttgtttaataatttacaggttcttaggctccatcctgtttgtatgaaattataatctgtggattggcctttaagcctgcattcttaacaaactcttcagttaattcttagatacactaaaaatctgagaaactctacatgtaactatttcttcagagtttgtcatatactgcttgtcatctgcatgtctactcagcatttgattaacatttgtgtaatatgaaataaaattacacagtaagtcatttaaccaaaaaaaaaaaaaaaa

SEQ ID NO: 4 - protein, 724 aa, Homo sapiens
MSFPKAPLKRFNDPSGCAPSPGAYDVKTLEVLKGPVSFQKSQRFKQQKESKQNLNVDKDTTLPASARKVKSSESKESQKNDKDLKILEKEIRVLLQERGAQDSRIQDLETELEKMEARLNAALREKTSLSANNATLEKQLIELTRTNELLKSKFSENGNQKNLRILSLELMKLRNKRETKMRGMMAKQEGMEMKLQVTQRSLEESQGKIAQLEGKLVSIEKEKIDEKSETEKLLEYIEEISCASDQVEKYKLDIAQLEENLKEKNDEILSLKQSLEENIVILSKQVEDLNVKCQLLEKEKEDHVNRNREHNENLNAEMQNLKQKFILEQQEREKLQQKELQIDSLLQQEKELSSSLHQKLCSFQEEMVKEKNLFEEELKQTLDELDKLQQKEEQAERLVKQLEEEAKSRAEELKLLEEKLKGKEAELEKSSAAHTQATLLLQEKYDSMVQSLEDVTAQFESYKALTASEIEDLKLENSSLQEKAAKAGKNAEDVQHQILATESSNQEYVRMLLDLQTKSALKETEIKEITVSFLQKITDLQNQLKQQEEDFRKQLEDEEGRKAEKENTTAELTEEINKWRLLYEELYNKTKPFQLQLDAFEVEKQALLNEHGAAQEQLNKIRDSYAKLLGHQNLKQKIKHVVKLKDENSQLKSEVSKLRCQLAKKKQSETKLQEELNKVLGIKHFDPSKAFHHESKENFALKTPLKEGNTNCYRAPMECQESWK

SEQ ID NO: 5 - protein, 14 aa, Artificial Sequence
  Synthetic Rhamm peptide mimetic that binds to hyaluronan
STMMRSHKTRSHHV

SEQ ID NO: 6 - protein, 9 aa, Homo sapiens
KIKHVVKLK

SEQ ID NO: 7 - protein, 8 aa, Artificial sequence
  Synthetic hyaluronan mimicking peptide 1
YDSEYESE

SEQ ID NO: 8 - protein, 8 aa, Artificial Sequence
  Synthetic Hyaluronan mimicking peptide 2
YDSEYESE

SEQ ID NO: 9 - protein, 8 aa, Artificial Sequence
  Synthetic Hyaluronan mimicking peptide 3
YDSEYESE

SEQ ID NO: 10 - protein, 12 aa, Artificial Sequence
  Synthetic anti-Rhamm Ab1 sequence
KSKFSENGNQKN

SEQ ID NO: 11 - protein, 12 aa, Artificial Sequence
  Synthetic anti-Rhamm Ab2 sequence
VSIEKEKIDEKS

SEQ ID NO: 12 - protein, 10 aa, Artificial Sequence
  Synthetic anti-Rhamm Ab3 sequence
QLRQQDEDFR

SEQ ID NO: 13 - protein, 12 aa, Artificial Sequence
  Synthetic hyaluronan scrambled control peptide
YLKQKKVKKHIV

SEQ ID NO: 14 - protein, 12 aa, artificial sequence
  Synthetic hyaluronan binding peptide
YKQKIKHVVKLK

(19) United States
(12) Patent Application Publication
(10) Pub. No.: US 2010/0062000 A1
(43) Pub. Date: Mar. 11, 2010

(54) Rhamm, a Co-Receptor and Its Interactions with Other Receptors in Cancer Cell Motility and the Identification of Cancer Prognitor Cell Populations

(75) Inventors: Eva A. Turley, London (CA); Mina J. Bissell, Berkeley, CA (US); Francoise Winnik, Westmount (CA)

Correspondence Address: LAWRENCE BERKELEY NATIONAL LABORATORY, Technology Transfer & Intellectual Propery Managem, One Cyolotron Road MS 56A-120, BERKELEY, CA, 94720, US

(73) Assignee: THE REGENTS OF THE UNIVERSITY OF CALIFORNIA, Oakland, CA (US)

(21) Appl. No.: 12/470,453

(22) Filed: May 21, 2009

Related U.S. Application Data
(63) Continuation-in-part of application No. PCT/US2007/085462, filed on Nov. 21, 2007.
(60) Provisional application No. 60/860,607, filed on Nov. 21, 2006.

(51) Int. Cl. A61K 39/395; C12Q 1/04; G01N 33/574; C12N 5/00; A61K 38/02; A61K 31/7052; C12Q 1/02; C12Q 1/68; A61P 35/00

(52) U.S. Cl. 424/172.1; 435/34; 435/7.23; 435/375; 514/2; 514/44 A; 435/29; 435/6

(57) ABSTRACT

CD44 is an integral hyaluronan receptor that can either promote or inhibit motogenic signaling in tumor cells. Rhamm is a non-integral cell surface (CD168) and intracellular hyaluronan binding protein that promotes cell motility in vitro and whose expression is strongly upregulated in aggressive tumors. The present invention describes compositions and methods for the prognosis and diagnosis of cancer by the detection of CD44/Rhamm complexes. The use of labeled Rhamm-binding agents in culture and in vivo to identify tumorigenic progenitor cells that exhibit an aggressive phenotype characterized by high Rhamm and CD44 expression is further described. Specific methods include using hyaluronan to target imaging or therapeutic agents to these progenitor tumor subsets in breast and likely other cancers.

### CROSS-REFERENCE TO RELATED APPLICATIONS

This application is a continuation-in-part of International Patent Application PCT/US2007/085462 filed on Nov. 21, 2007, which claims priority to U.S. Provisional Patent Application No. 60/860,607, filed on Nov. 21, 2006, and both of which are hereby incorporated by reference in its entirety. This application is also related to co-pending U.S. patent application Ser. No. 12/515,405 entitled “Modulation of Rhamm (CD168) for Selective Adipose Tissue Development,” which was filed 18 May 2009, which claims priority to International Patent Application PCT/PCT/US2007/085463, filed on 21 Nov. 2007, which claims priority to US. Provisional Patent application No. 60/860,606, all of which are hereby incorporated by reference in their entirety for all purposes.

### STATEMENT OF GOVERNMENTAL SUPPORT

This invention was made with government support under Grant number MOP-57694 awarded by the Canadian Institutes of Health Research (CIHR), under the Breast Cancer Society of Canada Translational Breast Cancer Research Unit, under Grant number DOD-PC050959 awarded by the U.S. Department of Defense, and under Contract No. DE-AC02-05CH11231 awarded by the U.S. Department of Energy. The government has certain rights in this invention.

### REFERENCE TO ATTACHED SEQUENCE LISTING

This application refers to and incorporates by reference the attached sequence listing found in paper and computer readable form.

### BACKGROUND OF THE INVENTION

1. Field of the Invention

The present invention relates to cancer prognosis and metastasis, more specifically it relates to hyaluronan binding protein interactions that affect cancer cell motility. The present invention further relates to the identification of cancer progenitor cell populations in the prognosis and diagnosis of cancer and other related diseases.

### RELATED ART

Cancer invasion and progression involves a motile/invasive cell phenotype, under complex regulation by growth factors/cytokines and extracellular matrix (ECM) components within the tumor microenvironment (Eccles, S. A., C. Box, and W. Court, Cell migration/invasion assays and their application in cancer drug discovery. Biotechnol Annu Rev, 2005. 11: p. 391-421; Entschladen, F., et al., Tumour-cell migration, invasion, and metastasis: navigation by neurotransmitters. Lancet Oncol, 2004. 5(4): p. 254-8; and Kenny, P. A., G. Y. Lee, and M. J. Bissell, Targeting the tumor microenvironment. Front Biosci, 2007. 12: p. 3468-74). Motogenic/invasion signaling in tumor cells can be stimulated by both paracrine and autocrine factors: the latter decrease the requirement of invasive carcinomas for stromal support (Keen, J. C. and N. E. Davidson, The biology of breast carcinoma. Cancer, 2003. 97(3 Suppl): p. 825-33; Muraoka-Cook, R. S., N. Dumont, and C. L. Arteaga, Dual role of transforming growth factor beta in mammary tumorigenesis and metastatic progression. Clin Cancer Res, 2005. 11(2 Pt 2): p. 937s-43s; Wells, A., Tumor invasion: role of growth factor-induced cell motility. Adv Cancer Res, 2000. 78: p. 31-101; Karnoub, A. E., et al., Mesenchymal stem cells within tumour stroma promote breast cancer metastasis. Nature, 2007. 449(7162): p. 557-63 and others).

Hyaluronan (HA, an anionic polymer of repeating units of glucuronic acid and N-acetylglucosamine) is one ECM component of stroma, that is associated with cancer (i.e. breast) progression: increased accumulation of tumor HA is prognostic of poor outcome in cancer patients (Edward, M., et al., Tumour regulation of fibroblast hyaluronan expression: a mechanism to facilitate tumour growth and invasion. Carcinogenesis, 2005. 26(7): p. 1215-23; Tammi, M. I., A. J. Day, and E. A. Turley, Hyaluronan and homeostasis: a balancing act. J Biol Chem, 2002. 277(7): p. 4581-4; Gotte, M. and G. W. Yip, Heparanase, hyaluronan, and CD44 in cancers: a breast carcinoma perspective. Cancer Res, 2006. 66(21): p. 10233-7). HA stimulates cancer cell motility in vitro, suggesting its importance in cancer cell invasion in vivo. Two HA receptors that have been implicated in cancer progression are CD44 and RHAMM (receptor for HA-mediated motility).

CD44 is a broadly expressed, type I integral cell surface membrane glycoprotein that participates in cell-cell and cell-matrix adhesions (Gotte, M. and G. W. Yip, Heparanase, hyaluronan, and CD44 in cancers: a breast carcinoma perspective. Cancer Res, 2006. 66(21): p. 10233-7; Sales, K. M., M. C. Winslet, and A. M. Seifalian, Stem Cells and Cancer: An Overview. Stem Cell Rev, 2007; Naor, D., et al., CD44 in cancer. Crit. Rev Clin Lab Sci, 2002. 39(6): p. 527-79). It is encoded as a single gene but exists as multiple isoforms that are generated by alternative splicing of 10 variable exons, as well as through post translational modifications (Naor, D., et al., CD44 in cancer. Crit. Rev Clin Lab Sci, 2002. 39(6): p. 527-79). The most commonly expressed CD44 isoform (the standard form or “CD44s”) is an 85 kDa protein that contains none of the variable exons. Originally described as the principal cell surface receptor for HA (Aruffo, A., et al., CD44 is the principal cell surface receptor for hyaluronate. Cell, 1990. 61(7): p. 1303-13), CD44 has since been shown to bind multiple ligands including fibronectin (Jalkanen, S, and M. Jalkanen, Lymphocyte CD44 binds the COOH-terminal heparin-binding domain of fibronectin. J Cell Biol, 1992. 116(3): p. 817-25) and osteopontin (Weber, G. F., S. Ashkar, and H. Cantor, Interaction between CD44 and osteopontin as a potential basis for metastasis formation. Proc Assoc Am Physicians, 1997. 109(1): p. 1-9). While the ability of each of the isoforms to bind HA and the downstream consequences of CD44/HA binding is incompletely understood, a role for CD44 as a receptor associated with motogenic functions of HA in breast cancer cell lines has been established in vitro (Tammi, M. I., A. J. Day, and E. A. Turley, Hyaluronan and homeostasis: a balancing act. J Biol Chem, 2002. 277(7): p. 4581-4.; Turley, E. A., P. W. Noble, and L. Y. Bourguignon, Signaling properties of hyaluronan receptors. J Biol Chem, 2002. 277(7): p. 4589-92; Toole, B. P., Hyaluronan: from extracellular glue to pericellular cue. Nat Rev Cancer, 2004. 4(7): p. 528-39; Adamia, S., C. A. Maxwell, and L. M. Pilarski, Hyaluronan and hyaluronan synthases: potential therapeutic targets in cancer. Curr Drug Targets Cardiovasc Haematol Disord, 2005. 5(1): p. 3-14). CD44 binds HA via an extracellular domain and its cytoplasmic tail activates intracellular signaling pathways that modify the cortical actin cytoskeleton (Naor, D., et al., CD44 involvement in autoimmune inflammations: the lesson to be learned from CD44-targeting by antibody or from knockout mice. Ann N Y Acad Sci, 2007. 1110: p. 233-47; Bourguignon, L. Y., CD44-mediated oncogenic signaling and cytoskeleton activation during mammary tumor progression. J Mammary Gland Biol Neoplasia, 2001. 6(3): p. 287-97; Marhaba, R. and M. Zoller, CD44 in cancer progression: adhesion, migration and growth regulation. J Mol Histol, 2004. 35(3): p. 211-31). Elevated CD44 has been correlated with both poor and good patient outcome suggesting that CD44 could enhance or suppress tumor growth and metastasis [e.g. (Watanabe, O., et al., Expression of a CD44 variant and VEGF-C and the implications for lymphatic metastasis and long-term prognosis of human breast cancer. J Exp Clin Cancer Res, 2005. 24(1): p. 75-82; Diaz, L. K., et al., CD44 expression is associated with increased survival in node-negative invasive breast carcinoma. Clin Cancer Res, 2005. 11(9): p. 3309-14)]. The basis for CD44 association with different outcomes in breast cancer patients is not completely understood but has been suggested to result from differential expression/function of CD44 isoforms in tumor cell subsets (Weber, G. F., et al., Absence of the CD44 gene prevents sarcoma metastasis. Cancer Res, 2002. 62(8): p. 2281-6; Abraham, B. K., et al., Prevalence of CD44+/CD24−/low cells in breast cancer may not be associated with clinical outcome but may favor distant metastasis. Clin Cancer Res, 2005. 11(3): p. 1154-9). The recent animal model evidence for a role of CD44 in tumor metastasis also suggests it this ECM receptor plays specific functions during different stages of tumorigenesis (Wicha, M. S., S. Liu, and G. Dontu, Cancer stem cells: an old idea—a paradigm shift. Cancer Res, 2006. 66(4): p. 1883-90; discussion 1895-6; Auvinen, P., et al., Expression of CD 44 s, CD 44 v 3 and CD 44 v 6 in benign and malignant breast lesions: correlation and colocalization with hyaluronan. Histopathology, 2005. 47(4): p. 420-8). Evidence for high expression of CD44 in tumor progenitor cells (also called tumor initiating cells) has suggested a new mechanism by which this receptor may promote tumor aggression. For example, CD44 expression in breast and other tumor cell subsets is associated with poor clinical outcome and the gene signature of tumor cells sorted for their high CD44 expression is also predictive of poor clinical outcome in breast and other cancers (Liu R, et al., 2007 N. Engl. J. Med 356, 217-26, Shipitsin M, et al., 2007, Cancer Cell, 11: 259-73). Since CD44 also facilitates signaling through other tumor cell-associated transmembrane receptors such as c-met and EGFR (Ponta, H., L. Sherman, and P. A. Herrlich, CD44: from adhesion molecules to signalling regulators. Nat Rev Mol Cell Biol, 2003. 4(1): p. 33-45; Florquin, S, and K. M. Rouschop, Reciprocal functions of hepatocyte growth factor and transforming growth factor-beta1 in the progression of renal diseases: a role for CD44? Kidney Int Suppl, 2003 (86): p. S15-20), the consequence of CD44 for tumor invasion and progression may also depend upon the proteins it associates with that modify its signaling properties, and vice versa.

Cell surface Rhamm (CD168, gene name Hmmr) is HA-binding protein/receptor that is not highly expressed in normal tissues but is commonly over-expressed in advanced cancers (Turley, E. A., P. W. Noble, and L. Y. Bourguignon, Signaling properties of hyaluronan receptors. J Biol Chem, 2002. 277(7): p. 4589-92; Toole, B. P., Hyaluronan: from extracellular glue to pericellular cue. Nat Rev Cancer, 2004. 4(7): p. 528-39), including breast cancer (Wang, C., et al., The overexpression of RHAMM, a hyaluronan-binding protein that regulates ras signaling, correlates with overexpression of mitogen-activated protein kinase and is a significant parameter in breast cancer progression. Clin Cancer Res, 1998. 4(3): p. 567-76; Assmann, V., et al., The pattern of expression of the microtubule-binding protein RHAMM/IHABP in mammary carcinoma suggests a role in the invasive behaviour of tumour cells. J Pathol, 2001. 195(2): p. 191-6; Pujana, M. A., et al., Network modeling links breast cancer susceptibility and centrosome dysfunction. Nat Genet, 2007. 39(11): p. 1338-1349). Rhamm hyperexpression or polymorphisms have been linked to poor outcome in many types of human tumors. (Ibid). For example, Rhamm mRNA hyperexpression is prognostic of poor outcome in breast cancer (Wang et al., 1998, Clin Cancer Res, 1998. 4(3): p. 567-76; Pujana et al., 2007, Nat Genet, 2007. 39(11): p. 1338-1349) while Rhamm polymorphisms are associated with susceptibility to this neoplastic disease (Pujana et al., 2007, Nat Genet, 2007. 39(11): p. 1338-1349). Rhamm hyperexpression in tumor cell subsets is not only associated with poor clinical outcome but significantly linked to increased metastatic spread (Wang et al., 1998, Cancer Res, 1998. 4(3): p. 567-76). Although Rhamm is expressed both as an intracellular and a cell surface protein [Turley, E. A., P. W. Noble, and L. Y. Bourguignon, Signaling properties of hyaluronan receptors. J Biol Chem, 2002. 277(7): p. 4589-92, Toole, B. P., Hyaluronan: from extracellular glue to pericellular cue. Nat Rev Cancer, 2004. 4(7): p. 528-39; Adamia, S., C. A. Maxwell, and L. M. Pilarski, Hyaluronan and hyaluronan synthases: potential therapeutic targets in cancer. Curr Drug Targets Cardiovasc Haematol Disord, 2005. 5(1): p. 3-14; Evanko, S. P., et al., Hyaluronan-dependent pericellular matrix. Adv Drug Deliv Rev, 2007. 59(13): p. 1351-65], its extracellular location has been most strongly linked with tumor progression [See Turley, E. A., P. W. Noble, and L. Y. Bourguignon, Signaling properties of hyaluronan receptors. J Biol Chem, 2002. 277(7): p. 4589-92,]. For example, in human AML, myelodysplastic syndrome and multiple myeloma [Schmitt, M., et al., RHAMM-R3 peptide vaccination in patients with acute myeloid leukemia, myelodysplastic syndrome and multiple myeloma elicits immunological and clinical responses. Blood, 2007, hereby incorporated by reference], Rhamm peptide vaccination reduces aggressive disease. A similar result was found in a mouse model of glioma [Amano, T., et al., Antitumor effects of vaccination with dendritic cells transfected with modified receptor for hyaluronan-mediated motility mRNA in a mouse glioma model. J Neurosurg, 2007. 106(4): p. 638-45]. Successful immune therapy such as reported in these studies requires de facto, display of Rhamm at the tumor cell surface. Collectively these results suggest that CD44 and Rhamm complexes are excellent therapeutic targets to control aggressive neoplastic disease, which are most likely contributed to by tumor progenitor cells.

Rhamm was first identified as an HA-dependent motility cell surface receptor (Hardwick, C., et al., Molecular cloning of a novel hyaluronan receptor that mediates tumor cell motility. J Cell Biol, 1992. 117(6): p. 1343-50) but is also an intracellular protein that interacts with interphase microtubules and the mitotic spindle, suggesting that it functions in multiple cell compartments (Maxwell, C. A., et al., RHAMM is a centrosomal protein that interacts with dynein and maintains spindle pole stability. Mol Biol Cell, 2003. 14(6): p. 2262-76; Assmann, V., et al., The intracellular hyaluronan receptor RHAMM/IHABP interacts with microtubules and actin filaments. J Cell Sci, 1999. 112 (Pt 22): p. 3943-54). Cell surface Rhamm promotes motility by affecting pp 60-c-src signal transduction (Hall, C. L., et al., pp 60(c-src) is required for cell locomotion regulated by the hyaluronanreceptor RHAMM. Oncogene, 1996. 13(10): p. 2213-24; Hall, C. L., F. S. Wang, and E. Turley, Src−/− fibroblasts are defective in their ability to disassemble focal adhesions in response to phorbol ester/hyaluronan treatment. Cell Commun Adhes, 2002. 9(5-6): p. 273-83) but is also involved in sustaining ERK1,2 activation by growth factors (Zhang, S., et al., The hyaluronan receptor RHAMM regulates extracellular-regulated kinase. J Biol Chem, 1998. 273(18): p. 11342-8; Hamilton, S. R., et al., The hyaluronan receptors CD44 and Rhamm (CD168) form complexes with ERK1,2 that sustain high basal motility in breast cancer cells. J Biol Chem, 2007. 282(22): p. 16667-80; Tolg, C., et al., Rhamm−/− fibroblasts are defective in CD44-mediated ERK1,2 motogenic signaling, leading to defective skin wound repair. J Cell Biol, 2006. 175(6): p. 1017-28). Thus, Rhamm may regulate the intensity and duration of growth, motility, and survival signals. We have recently shown that Rhamm can substitute for CD44 in promoting cell migration ansion in vitro and in vivo (Nedvetzki, S., et al., RHAMM, a receptor for hyaluronan-mediated motility, compensates for CD44 in inflamed CD44-knockout mice: a different interpretation of redundancy. Proc Natl Acad Sci USA, 2004. 101(52): p. 18081-6), suggesting that these two HA-binding proteins are functionally linked under certain conditions.

In addition to cell-autonomous tumor progression events, cancer cells are sensitive to exogenous factors in their microenvironment (Kenny, P. A., G. Y. Lee, and M. J. Bissell, Targeting the tumor microenvironment. Front Biosci, 2007. 12: p. 3468-74), including cytokines/growth factors and extracellular matrix components such as HA (Hall, C. L., et al., Hyaluronan and the hyaluronan receptor RHAMM promote focal adhesion turnover and transient tyrosine kinase activity. J Cell Biol, 1994. 126(2): p. 575-88; Sohara, Y., et al., Hyaluronan activates cell motility of v-Src-transformed cells via Ras-mitogen-activated protein kinase and phosphoinositide 3-kinase-Akt in a tumor-specific manner. Mol Biol Cell, 2001. 12(6): p. 1859-68) that regulate intracellular signaling important to development of malignant characteristics. These factors act coordinately with activating mutations in critical signal transduction pathways to modify tumor cell behavior (Wang, F., et al., Phenotypic reversion or death of cancer cells by altering signaling pathways in three-dimensional contexts. J Natl Cancer Inst, 2002. 94(19): p. 1494-503). A pathway that regulates migration and invasion, including that of breast cancer cells, is the Ras/Raf/MEK1,2/ERK1,2 cascade (Roberts, P. J. and C. J. Der, Targeting the Raf-MEK-ERK mitogen-activated protein kinase cascade for the treatment of cancer. Oncogene, 2007. 26(22): p. 3291-310; Johnston, S. R., Targeting downstream effectors of epidermal growth factor receptor/HER2 in breast cancer with either farnesyltransferase inhibitors or mTOR antagonists. Int J Gynecol Cancer, 2006. 16 Suppl 2: p. 543-8). ERK1,2 are MAP kinases activated in pathways induced by growth factor and ECM receptor signaling, and are often constitutively active in human tumors including breast cancers (Reddy, K. B., S. M. Nabha, and N. Atanaskova, Role of MAP kinase in tumor progression and invasion. Cancer Metastasis Rev, 2003. 22(4): p. 395-403; Ferrer-Soler, L., et al., An update of the mechanisms of resistance to EGFR-tyrosine kinase inhibitors in breast cancer: Gefitinib (Iressa)-induced changes in the expression and nucleo-cytoplasmic trafficking of HER-ligands (Review). Int J Mol Med, 2007. 20(1): p. 3-10). Invasive breast cancer cell lines (including MDA-MB-231) have high basal ERK1,2 activity in contrast to lower levels in less invasive cell lines (including MCF7). Sustained ERK1,2 activity is also required for enhanced tumor cell motility and invasion in vitro (Hamilton, S. R., et al., The hyaluronan receptors CD44 and Rhamm (CD168) form complexes with ERK1,2 that sustain high basal motility in breast cancer cells. J Biol Chem, 2007. 282(22): p. 16667-80). The mechanism behind sustained ERK1,2 activation and its link with downstream tumor cell motility events are poorly understood (Pouyssegur, J., V. Volmat, and P. Lenormand, Fidelity and spatio-temporal control in MAP kinase (ERKs) signalling. Biochem Pharmacol, 2002. 64(5-6): p. 755-63; Torii, S., et al., Regulatory mechanisms and function of ERK MAP kinases. J Biochem (Tokyo), 2004. 136(5): p. 557-61; Kermorgant, S, and P. J. Parker, c-Met signalling: spatio-temporal decisions. Cell Cycle, 2005. 4(3): p. 352-5). The concept that tumors arise from progenitor cells, e.g. breast tumors, is a developing field that has not yet been incorporated into clinical treatment regimes. In particular the notion that only a small subset of neoplastic cells within a primary tumor has tumorigenic potential (e.g. can give rise to another tumor when transplanted to another animal or tissue) is a new concept for cancer, particularly for breast cancer.

Recent evidence suggests that the majority of “tumor initiating activity” in breast cancer resides in a small population (1-2%) of progenitor cells characterized by high expression of the HA receptor CD44 amongst other surface characteristics (Al-Hajj, M., M. S. Wicha, A. Benito-Hernandez, S. J. Morrison, and M. F. Clarke, 2003. Prospective identification of tumorigenic breast cancer cells. Proc Natl Acad Sci USA. 100(7): p. 3983-8). We have reported that the presence of a similarly small subset of breast cancer cells that overexpress another HA (HA) receptor, Rhamm, is prognostic of poor patient outcome and associated with enhanced peripheral metastases (Wang, C., A. D. Thor, D. H. Moore, 2nd, Y. Zhao, R. Kerschmann, R. Stern, P. H. Watson, and E. A. Turley, 1998. The overexpression of RHAMM, a hyaluronan-binding protein that regulates ras signaling, correlates with overexpression of mitogen-activated protein kinase and is a significant parameter in breast cancer progression. Clin Cancer Res. 4(3): p. 567-76). See also Collis, L., C. Hall, L. Lange, M. Ziebell, R. Prestwich, and E. A. Turley, 1998. Rapid hyaluronan uptake is associated with enhanced motility: implications for an intracellular mode of action. FEBS Lett. 440(3): p. 444-9.

In addition, a new area of research suggests that bone marrow stem cells are promoted to traffic in response to cytokine/growth factor mixtures produced by primary tumors and that the specific combinations of these tumor-derived stimulatory factors direct the normal host stem cells to tissue compartments. These cells then modify the microniche of the tissue so as to favor neoplastic colonization of this tissue (Psaila, B, Kaplan R N, Port E R, and Lyden D. 2006. Priming the “soil” for breast cancer metastasis: the premetastatic niche. Breast Dis. 26: 65-74). Rhamm has recently been reported to be expressed by both embryonic stem cells and bone marrow stem cells and that this expression is both required for trafficking and stem cell division (Choudhary, M., et al., Putative Role of Hyaluronan and its related genes, HAS2 and RHAMM in Human Early Preimplantation Embryogenesis and Embryonic Stem Cell Characterisation. Stem Cells, 2007.). Thus tumor progenitor cells and host stem cells likely coordinate tumor spread to distant tissues.

Thus, there is a need for methods of assessing the importance of malignant progenitors and their collaboration with normal stem cells to patient outcome; this would allow clinicians to sort patients with primary cancers into those at risk for metastases and those not or less likely to metastasize. In turn this sorting would permit appropriate tailoring of therapeutic intervention for each patient based upon their risk for metastases. Furthermore, methods for identifying tumor progenitor cells will ultimately lead to specific intervention methods and imaging methods that permit tracking response to treatment.

### SUMMARY OF THE INVENTION

The present invention provides a method for identifying cancer progenitor cells, which comprises providing a sample and detecting the presence of CD44/Rhamm complexes, wherein if the sample contains the CD44/Rhamm complexes, this indicates that the sample contains cancer progenitor cells.

In one embodiment, the presence of CD44/Rhamm complexes is detected by contacting the sample with a probe (e.g. hyaluronan, peptide fragment or Rhamm-binding peptide mimetics or small chemical mimics) that specifically binds to the CD44/Rhamm complex. Optionally, in some embodiments, this probe may be labeled with a detectable marker to allow detection of the location of the cancer progenitor cell. The probe can include a small molecule, a monoclonal antibody, recombinant proteins, antisense nucleic acids, peptides and peptide mimetics.

In other embodiments, the presence of CD44/Rhamm complexes is detected by contacting the sample with a first probe that specifically binds to CD44 or a Rhamm mimic (e.g. peptide mimetic or small chemical mimetic, hyaluronan or hyaluronan fragment) and a second probe that binds to Rhamm or a CD44 mimic (e.g. peptide mimetic or small chemical mimetic, hyaluronan or hyaluronan fragment). The complex is then imaged, whereby the detection of CD44/Rhamm complexes indicates a tumor-progenitor cell.

In one embodiment, a method for prognosing cancer in vivo comprising detecting in a subject the presence of CD44/Rhamm complexes by administering a probe (e.g. hyaluronan, peptide fragment or Rhamm mimetic peptides, or small molecules) that specifically binds to CD44/Rhamm complexes to indicate the presence of a cancer progenitor cell. Optionally, in some in vivo embodiments, the probe may be labeled with a detectable marker (e.g. Gadolinium, gold and other metals, I¹²³, near far red fluorochromes) to allow imaging and detection of the in vivo location of cancer progenitor cells.

In other embodiments, the presence of CD44/Rhamm complexes is detected by contacting a sample with a first antibody or Rhamm mimic (e.g. peptide mimetic or small chemical mimetic) that specifically binds to CD44 and a second antibody or CD44 mimic (e.g. peptide mimetic or small chemical mimetic) that binds to Rhamm.

The present invention also provides a method for detecting cancer comprising providing a cell or tissue sample and detecting the presence or absence of CD44/Rhamm complexes, whereby the presence of the CD44/Rhamm complexes indicates the presence of an aggressively metastatic cancer cell.

The present invention further provides a method for inhibiting cancer cell metastasis comprising contacting a cancer cell with a compound that inhibits formation of CD44/Rhamm complex.

In some embodiments, the sample is taken from a mammal. In a preferred embodiment the mammal is a human. In some preferred embodiments, the sample is a tissue sample suspected to be contain breast cancer, colon cancer, gastric cancer, gliomas, and other parenchymal tumors; leukemias, multiple myeloma and other immune-cell related tumors; desmoid and other mesenchymal related tumors and skin cancers such as basal cell carcinoma and melanoma.

In some embodiments, the compound that inhibits formation of CD44/Rhamm complexes is an antibody, a small molecule, a peptide, a mimetic, a siRNA, an antisense oligo, or an aptamer. In other embodiments, the compound that inhibits formation of CD44/Rhamm complex is an antibody that specifically binds CD44 or an antibody that specifically binds Rhamm.

Additionally, the present invention provides a method for identifying compounds that inhibit cancer cell metastasis. The method comprises contacting a cancer cell with a compound suspected of inhibiting metastasis of cancer cells and detecting CD44/Rhamm complexes, whereby a reduction in the amount of CD44/Rhamm complexes identifies the compound as an inhibitor of cancer cell metastasis.

In some embodiments, the compound suspected of being an inhibitor is an antibody, a small molecule, a peptide, a mimetic, a siRNA, an antisense oligo, or an aptamer.

### BRIEF DESCRIPTION OF THE FIGURES

FIG. 1. MDA-MB-231 cells produce higher levels of HA than MCF7 cells and rely upon HA to promote high basal motility. A: MDA-MB-231 cells produced significantly higher levels of HA than MCF7 cells. Values represent the Mean and S.E.M., N=2 from 1 of 3 similar experiments. B: A 12-mer HA binding peptide significantly reduced the motility of the MDA-MB-231 cells when compared to controls, where as it had no effect on the motility of the MCF7 cells. Values represent the Mean and S.E.M., N=30 cells from 1 of 3 similar experiments. C: Exogenous HA (25-50 μg/ml) was significantly stimulated the motility of serum-starved MDA-MB-231 cells when compared to MCF7 cells, which did not respond to HA. Values represent the Mean and S.E.M., N=30 cells from 1 of 3 similar experiments.

FIG. 2. Cell surface and total cellular CD44 protein expression is higher in the aggressive MDA-MB-231 breast cancer cell line than in the less aggressive MCF7 cell line. A: Western blot analysis and densitometric quantification (normalized to total cellular protein) of total cellular CD44s protein levels in breast cancer cell lines. Constitutive expression of CD44s protein was significantly higher in MDA-MB-231 cells when compared to MCF7. Values represent the Mean and S.E.M., N=3 experiments. B: Flow cytometry analysis of cell surface CD44s expression in invasive (MDA-MB-231) and non-invasive (MCF7) breast cancer cell lines. MDA-MB-231 cells had higher levels of cell surface CD44 when compared to MCF7 cells. Similar trends were observed for Ras-MCF10A and parental MCF10A cells (data not shown). Values represent 1 of 3 similar experiments.

FIG. 3. Total cellular Rhamm protein expression is higher in aggressive breast cancer cell lines than in less aggressive cell lines. A: Diagram of Rhamm protein forms outlining areas of reactivity for the different Rhamm antibodies. B: Constitutive expression of total cellular Rhamm protein was higher in MDA-MB-231 cells compared to MCF7 cells. Rhamm isoforms expressed in MCF7 cells consisted primarily of the 85kD (full-length) and 43kD isoforms (Ab-2) where as MDA-MB-231 cells expressed an additional isoform of 63 kD (Ab-2). The quantification of Rhamm protein expression was determined by calculating the densitometric ratios of each of the protein isoforms/total Rhamm protein (obtained by totaling the densitometric values of each Rhamm immunoreactive band recognized by Ab-2). Values represent the Mean and S.E.M., N=3 experiments.

FIG. 4. Cell surface Rhamm expression is higher in aggressive breast cancer cell lines than in less aggressive cell lines. Flow cytometry analysis of cell surface Rhamm expression in invasive (MDA-MB-231) and non-invasive (MCF7) breast cancer cell lines. MDA-MB-231 cells had higher levels of cell surface Rhamm when compared to MCF7 cells. Further, MDA-MB-231 cells has all Rhamm isoforms (43, 63, and 85 kDa) on their cell surface. Similar trends were observed for Ras-MCF10A and parental MCF10A cells (data not shown). Values represent 1 of 3 similar experiments.

FIG. 5. Subcellular distribution of CD44 and Rhamm in breast cancer cell lines. A: Confocal analysis shows that MCF7 cells expressed high levels of Rhamm as a cytoplasmic protein (red fluorescence, a, arrow, c) but no detectable levels of CD44 protein (green fluorescence, b,c) when compared to IgG control (d). In contrast, in the MDA-MB-231 cells CD44 (f,g) and Rhamm (e,g) co-localized (yellow, white enhancement in inset panels) primarily within perinuclear vesicular structures (g, arrow) when compared to IgG control (h). DAPI (blue stain) was used to detect the nuclei. Representative micrographs from 1 of 4 similar experiments are shown. B: Anti-Rhamm antibodies immunoprecipitated two CD44 isoforms (approximately 85 and 120 kD) in MDA-MB-231 and Ras-MCF10A cells but not in MCF7 and parental MCF10A cells. N=3 experiments. C: Anti-CD44 antibodies immunoprecipitated Rhamm in all of the cell lines tested. Specifically, the 63 and 85 kDa isoforms were immunoprecipitated in the aggressive MDA-MB-231 and Ras-MCF10A cell lines. They were also both immunoprecipitated in the less aggressive MCF7 cells, though to a much lesser extent that in the MDA-MB-231 cells. Only the 85 kDa Rhamm isoform was immunoprecipitated in the parental MCF10A cells. N=3 experiments. D: Recombinant Rhamm-GST (63 kDa isoform) protein pulled down CD44s and a variant form from MDA-MB-231 lysates. GST recombinant protein was used as control. N=4 experiments.

FIG. 6. MDA-MB-231 and Ras-MCF10A tumor cells exhibit rapid motility that is CD44 and Rhamm-dependent A: Basal motility rates of MDA-MB-231 and Ras-MCF10A were significantly higher than that of MCF7 and parental MCF10A cells. Values represent the Mean and S.E.M., N=30 cells from 1 of 3 similar experiments. B: Anti-Rhamm or anti-CD44 antibodies significantly reduced the motility of the MDA-MB-231 and Ras-MCF10A cells, while they had no effect on the basal motility rates of the MCF7 or MCF10A cells (data not shown). The addition of both blocking antibodies in combination did not have an additive effect. Values represent the Mean and S.E.M., N=30 cells from 1 of 3 similar experiments.

FIG. 7. ERK1,2 expression and activation in breast tumor cell lines. A: Constitutive expression of total ERK1,2 protein was significantly higher in MDA-MB-231 and Ras-MCF10A cells when compared to MCF7 and parental MCF10A cells. Values represent the Mean and S.E.M., N=3 experiments. B: Basal levels of phospho-ERK1,2 were significantly higher in MDA-MB-231 cells when compared to MCF7 cells (0 minutes). Levels of phospho-ERK1,2 did not significantly change in MDA-MB-231 cells in response to 20 ng/mL EGF stimulation where as MCF7 cells did respond with maximum activation at 10 minutes and a return to baseline by 30 minutes post-stimulation. Values represent the Mean and S.E.M., N=3 from 1 of 3 similar experiments.

FIG. 8. CD44 and Rhamm co-localize and co-immunoprecipitate with active-ERK1,2. A: Confocal analysis: MCF7 cells expressed no detectable CD44 (a,c) and only low levels of phospho-ERK1,2 (b,c) when compared to the IgG control (d). In contrast, in the MDA-MB-231 cells CD44 (e,g) and phospho-ERK1,2 (f,g) co-localized (yellow, white enhancement in inset panels) as vesicular structures in the perinucleus (g, arrow) and, to a more limited extent, in the nucleus (g) when compared to the IgG control (h). DAPI (blue stain) was used to detect the nuclei. Representative micrographs from 1 of 4 similar experiments are shown. B: Anti-Rhamm antibodies immunoprecipitated ERK1,2 in all cell lines (the invasive MDA-MB-231 and Ras-MCF10A cells and non-invasive MCF7 and parental MCF10A cells). N=3 experiments. C: Anti-ERK1,2 antibodies immunoprecipitated the 63 kD Rhamm isoform in all cell lines (the invasive MDA-MB-231 and Ras-MCF10A cells and non-invasive MCF7 and parental MCF10A cells). The 85 kD full-length Rhamm form was not immunoprecipitated. N=3 experiments. D: Recombinant Rhamm-GST protein (63 kDa and 43 kDa isoforms) were able to pull down ERK1,2 protein from MDA-MB-231 lysates. GST recombinant protein was used as control. N=3 experiments.

FIG. 9. Anti-Rhamm and anti-CD44 antibodies reduce ERK1,2 activity and ERK1,2-dependent motility of MDA-MB-231 cells. A: Anti-Rhamm or anti-CD44 antibodies reduced the levels of phospho-ERK1,2 in MDA-MB-231 while they had no effect on phospho-ERK1,2 levels in MCF7 (data not shown). The addition of both blocking antibodies in combination did not have an additive effect. Values represent the Mean and S.E.M., N=3 experiments. B: The addition of the MEK1 inhibitor, PD098059 significantly reduced the motility of the MDA-MB-231 cells while it had no effect on the motility of the MCF7 cells. The combination of PD098059 and anti-Rhamm antibodies had no additive effect on MDA-MB-231 cell motility. Values represent the Mean and S.E.M., N=30 cells from 1 of 3 similar experiments.

FIG. 10. Total cellular CD44 and Rhamm protein expression is higher in the aggressive Ras-MCF10A breast cancer cell line than in the less aggressive, parental MCF10A cell line. A: Western blot analysis and densitometric quantification (normalized to total cellular protein) of total cellular CD44s protein levels in breast cancer cell lines. Constitutive expression of CD44s protein was significantly higher in Ras-MCF10A cells when compared to the parental MCF10A cells. Values represent the Mean and S.E.M., N=3 experiments. B: Constitutive expression of total Rhamm protein was higher in Ras-MCF10A cells when compared to parental MCF10A cells. However, similar to the MDA-MB-231 cells, Ras-MCF10A cells expressed three predominant Rhamm isoforms (85, 63, and 43 kD) whereas the parental MCF10A cells expressed much lower levels of all three isoforms. Values represent the Mean and S.E.M., N=3 experiments.

FIG. 11. High expression of Rhamm and CD44 are associated with rapid uptake of HA. A. confocal images and heat maps of Texas-red HA. B. Quantification of uptake.

FIG. 12A: CD44 antibodies block uptake of Texas Red-HA A. confocal analysis of HA-uptake with (a,b) and without (c,d) CD44 antibodies. B and C: Graphs showing the mean fluorescence intensity of Rhamm transfected using confocal analysis. Uptake of HA is blocked by CD44 antibody and expression of Rhamm mutants versus a control.

FIG. 14. Gadolinium-labelled HA for use in MRI imaging. A: Schematic for modification of HA with gadolinium. B: Graph showing comparison of GD-HA uptake in MDA-MB-231 cells (RHAMM/CD44 high) versus MCF-7 cells (RHAMM/CD44 low). C: graph showing the differential uptake of GD-HA in xenografts of MDA-MB-231 and MCF-7 breast tumor cells. D: Visualization of MDA-MD-231 xenografts (tumor) in nude mice which express HA receptors CD44 and Rhamm. Liver expresses HA receptor HARE, and is therefore also positive.

FIG. 15A, B: Examples of Rhamm binding and hyaluronan peptide mimetics.

FIG. 16. Serum hyaluronan levels following I.V. injection of 10 mg/kg. Values are μg/ml).

FIG. 17. Size of unlabeled HA (4-12 mers and full length) required to block uptake of full length Texas red HA by CD44++/Rhamm++ expressing cells. A. confocal analysis of Texas red HA uptake and quantification of this uptake. B. Uptake of Texas Red alone (a), Texas-Red HA Depolymerized with hyaluronidase (b) and cells photographed with no treatment (c, control).

FIG. 18. Specific sizes of HA are required for rapid uptake into cells and these sizes promote bridging between CD44 and Rhamm. Upper panel: Sized Texas Red-labeled-HA fragments (960 Da, 2.8 kDa, 8.4 kDa, 14 kDa, 50 kDa and 220 kDa) were added to cultured breast cancer cells to determine the smallest HA fragment required for uptake into aggressive tumor cells. The most rapid uptake occurred between 8.4 kDa and 50 kDa. Lower panel: The ability of these fragments to bind to or bridge both Rhamm and CD44 was assessed using sized HA fragments linked to sepharose beads, which were used to “pull-down” Rhamm and CD44 expressed by MDA-MB-231 breast tumor cells. Small fragments bind to Rhamm only but not to CD44 while the 8.4 (not shown) or 50 kDa HA (shown) binds to and bridges both receptors. These results show that the HA fragments, which are most rapidly internalized, bind to both CD44 and Rhamm.

FIG. 19. HA mimetics that bind to Rhamm are more rapidly internalized in progenitor-like MDA-MB-231 than in MCF7 breast tumor cells. An HA mimetic peptide was labeled with Texas Red and added (10 ug/ml) to sparse cultures of breast tumor cells. The peptide was taken up more rapidly and to a greater extent in the progenitor-like MDA-MB-231 tumor cells relative to MCF-7 breast tumor cells.

FIG. 20. GD-HA uptake was greater in MDA-MB-231 than in MCF tumor cell aggregates.

FIG. 21. FE-HA uptake into MDA-MB-231 tumor xenografts.

FIG. 22. Au-HA uptake into tumor cells.

FIG. 23. Flow cytometry of HA uptake into breast tumor cells.

FIG. 24. Cy5.5-HA uptake into calcein green marked MDA-MB-231 cells (arrows).

FIG. 25. Microsphere-HA bind in a linear manner to recombinant Rhamm.

FIG. 26. Soluble HA competes with microsphere HA for binding to recombinant RHAMM.

FIG. 27. Quantification of CD44, CD24 and HA.

FIG. 28. The relationship amongst CD44 expression, tumor subtype, CD24−/CD44 phenotype and Cy5.5-HA uptake.

FIG. 29. Uptake HA into breast cancer cell lines representing different breast cancer molecular subtypes.

FIG. 30. Flow cytometry showing subpopulations with different HA uptake.

FIG. 31. Uptake of Texas Red HA was found to be saturable (data not shown), and HA is size dependent and is competed for by soluble unlabeled HA (FIGS. 31A and B) indicating that it is receptor mediated.

FIGS. 32 and 33. Serum levels of hyaluronan after i.v. or subcutaneous injection or oral gavage.

FIGS. 34 and 35 show FACS analysis of MDA-mb-231 cells.

### DETAILED DESCRIPTION OF THE INVENTION

### I. Introduction

The present invention provides a method for identifying cancer progenitor subsets. The present invention also provides a method for prognosing cancer. Further, a method for inhibiting cancer cell metastasis by regulating cancer cell motility has been provided. Additionally, the present invention provides a method for identifying compounds that inhibit cell metastasis.

The present invention is based on the surprising discovery of the relationship of Rhamm as a co-receptor and its interactions with other cell surface receptors. Specifically, disclosed for the first time herein is an association between Rhamm and CD44. We obtained evidence that Rhamm and CD44 associate as indicated by co-immunoprecipitation and co-localization data. Both Rhamm and CD44 have binding affinity for a natural ligand, hyaluronan, a polysaccharide with a repearting disaccharide. Thus, we describe an autocrine mechanism by which invasive human breast cancer cell lines sustain high basal motility. This mechanism requires endogenous hyaluronan synthesis and the formation of Rhamm/CD44/ERK1,2 complexes.

Highly motile and invasive MDA-MB-231 and Ras-MCF10A cells produce higher levels of endogenous hyaluronan, CD44 and cell surface Rhamm, and exhibit higher basal activation of ERK1,2 than less migratory MCF7 and MCF10A breast cell lines. The enhanced motility of the more invasive cell lines depends on the interaction of hyaluronan with cells, the activation of ERK1,2 and the participation of both CD44 and cell surface Rhamm. Furthermore, MDA-MB-231 tumor cells have been shown to exhibit a Basal “B’ or breast tumor progenitor phenotype while MCF-7 breast tumor cells have been classified as an example of a luminal epithelial tumor phenotype (Neve R M, et al., 2006 Cancer Cell 10: 515-527).

Combinations of anti-CD44, anti-Rhamm antibodies and a MEK1 inhibitor (PD098059) have less-than-additive blocking effects, suggesting action of all three proteins on a common motogenic signaling pathway. Rhamm, CD44 and ERK1,2 proteins uniquely co-immunoprecipitate and colocalize in vesicular structures in the perinuclear region of highly motile breast cancer cell lines. CD44/Rhamm complexes are not evident in less invasive cell lines, suggesting that the effect of CD44 on tumor cell motility may depend in part on its ability to partner with other hyaluronan receptors like Rhamm.

Collectively, these results suggest that cell surface Rhamm and CD44 act together in a hyaluronan dependent mechanism to coordinate sustained signaling through ERK1,2 leading to high basal rates of motility of invasive breast cancer cells.

### II. Abbreviations

“2D” 2-dimensional culture

“3D” 3-dimensional culture

“bFGF-2” Basic fibroblast growth factor-2

“CD44” is an integral hyaluronan receptor that can either promote or inhibit motogenic signaling in tumor cells.

“CD168” another name for Rhamm

“ECM” Extracellular matrix

“ERK1,2” Extracellular regulated kinase 1,2

“FAK” Focal adhesion kinase

“HA” Hyaluronic acid/Hyaluronan

“Matrigel” Basement membrane matrix

“MEFs” Mouse embryonic fibroblasts

“Mek1” Mitogen activated kinase kinase 1

“MMPs” Matrix metalloproteinases

“Motogen,” a factor that increases the motility of cancer cells.

“MW” Molecular weight

“MW_{avg}” Average molecular weight

“PDGF-BB” Platelet derived growth factor-BB

“PDGFR” Platelet derived growth factor receptor

“PMNs” polymorphonuclear cells

“Rhamm” Receptor for Hyaluronic Acid Mediated Motility, also known as CD168. Rhamm is a non-integral cell surface (CD168) and intracellular hyaluronan binding protein that promotes cell motility in vitro and whose expression is strongly upregulated in aggressive tumors.

“Rh^{Fl}” Full-length Rhamm

“Rh−/−” Rhamm−/−

“TE” Tris-EDTA

“TGF-β” Transforming growth factor-β

“TGF-βR” Transforming growth factor-β receptor

“Wt” Wild-type

### III. Description of Rhamm and its Co-Factors

HA: Hyaluronan (“HA”) was originally proposed to be an autocrine motility factor for fibrosarcoma tumor cells (Turley, E. A., Molecular mechanisms of cell motility. Cancer Metastasis Rev, 1992. 11(1): p. 1-3) and high endogenous production of HA has since been shown to provide autocrine motility signals in embryonic cells (Choudhary, M., et al., Putative Role of Hyaluronan and its related genes, HAS2 and RHAMM in Human Early Preimplantation Embryogenesis and Embryonic Stem Cell Characterisation. Stem Cells, 2007; Camenisch, T. D., et al., Disruption of hyaluronan synthase-2 abrogates normal cardiac morphogenesis and hyaluronan-mediated transformation of epithelium to mesenchyme. J Clin Invest, 2000. 106(3): p. 349-60; Bakkers, J., et al., Has2 is required upstream of Rac1 to govern dorsal migration of lateral cells during zebrafish gastrulation. Development, 2004. 131(3): p. 525-37), hematopoietic progenitor cells (Pilarski, L. M., et al., Potential role for hyaluronan and the hyaluronan receptor RHAMM in mobilization and trafficking of hematopoietic progenitor cells. Blood, 1999. 93(9): p. 2918-27), and a variety of human tumor cells (Sales, K. M., M. C. Winslet, and A. M. Seifalian, Stem Cells and Cancer: An Overview. Stem Cell Rev, 2007; Naor, D., et al., CD44 in cancer. Crit. Rev Clin Lab Sci, 2002. 39(6): p. 527-79). Specifically, HA as an autocrine motility factor that is produced by, and required for, motility of aggressive breast cancer cells is attractive given the close relationship of HA production/HAS expression (Tammi, M. I., A. J. Day, and E. A. Turley, Hyaluronan and homeostasis: a balancing act. J Biol Chem, 2002. 277(7): p. 4581-4; Toole, B. P., Hyaluronan: from extracellular glue to pericellular cue. Nat Rev Cancer, 2004. 4(7): p. 528-39; Udabage, L., et al., The over-expression of HAS2, Hyal-2 and CD44 is implicated in the invasiveness of breast cancer. Exp Cell Res, 2005. 310(1): p. 205-17) and co-localization of HA with CD44 in later stages of breast and other cancers (Auvinen, P., et al., Expression of CD 44 s, CD 44 v 3 and CD 44 v 6 in benign and malignant breast lesions: correlation and colocalization with hyaluronan. Histopathology, 2005. 47(4): p. 420-8), and the prognostic value of elevated HA in peri-tumor stroma or tumors themselves, as a marker for poor outcome in this disease (Toole, B. P., T. N. Wight, and M. I. Tammi, Hyaluronan-cell interactions in cancer and vascular disease. J Biol Chem, 2002. 277(7): p. 4593-6). HA has consistently been demonstrated to activate motogenic signaling through Ras (Camenisch, T. D., et al., Disruption of hyaluronan synthase-2 abrogates normal cardiac morphogenesis and hyaluronan-mediated transformation of epithelium to mesenchyme. J Clin Invest, 2000. 106(3): p. 349-60; Bakkers, J., et al., Has2 is required upstream of Rac1 to govern dorsal migration of lateral cells during zebrafish gastrulation. Development, 2004. 131(3): p. 525-37; Hall, C. L., et al., Overexpression of the hyaluronan receptor RHAMM is transforming and is also required for H-ras transformation. Cell, 1995. 82(1): p. 19-26) and to require activation of ERK1,2 and PI 3-kinase/AKT to promote motility (Toole, B. P., Hyaluronan: from extracellular glue to pericellular cue. Nat Rev Cancer, 2004. 4(7): p. 528-39; Sohara, Y., et al., Hyaluronan activates cell motility of v-Src-transformed cells via Ras-mitogen-activated protein kinase and phosphoinositide 3-kinase-Akt in a tumor-specific manner. Mol Biol Cell, 2001. 12(6): p. 1859-68; Goueffic, Y., et al., Hyaluronan induces vascular smooth muscle cell migration through RHAMM-mediated PI3K-dependent Rac activation. Cardiovasc Res, 2006. 72(2): p. 339-48).

CD44: CD44 is an integral hyaluronan receptor that can either promote or inhibit motogenic signaling in tumor cells. Although the HA receptors CD44 and cell surface Rhamm (CD168) have been shown to mediate HA regulation of a Ras-controlled motogenic pathway, the majority of studies have focused on the exclusive role of CD44. An overwhelming number support a major role for CD44 in promoting aggressive breast cancer behavior, including cell motility in vitro and in experimental tumor models as demonstrated by use of CD44 antibodies, blocking CD44 or HA fragments, and genetic deletion or knockdown of this HA receptor (Weber, G. F., S. Ashkar, and H. Cantor, Interaction between CD44 and osteopontin as a potential basis for metastasis formation. Proc Assoc Am Physicians, 1997. 109(1): p. 1-9; Toole, B. P., Hyaluronan: from extracellular glue to pericellular cue. Nat Rev Cancer, 2004. 4(7): p. 528-39; Udabage, L., et al., The over-expression of HAS2, Hyal-2 and CD44 is implicated in the invasiveness of breast cancer. Exp Cell Res, 2005. 310(1): p. 205-17). Nevertheless, reports have also documented that increased motility or invasion of breast and other tumor cells is associated with either shedding of CD44 [e.g. (Goueffic, Y., et al., Hyaluronan induces vascular smooth muscle cell migration through RHAMM-mediated PI3K-dependent Rac activation. Cardiovasc Res, 2006. 72(2): p. 339-48)], genetic loss or blocking of CD44 functions (Goueffic, Y., et al., Hyaluronan induces vascular smooth muscle cell migration through RHAMM-mediated PI3K-dependent Rac activation. Cardiovasc Res, 2006. 72(2): p. 339-48; Lopez, J. I., et al., CD44 attenuates metastatic invasion during breast cancer progression. Cancer Res, 2005. 65(15): p. 6755-63). These experimental discrepancies that reveal a capacity of CD44 for both promoting and inhibiting tumor cell behavior such as motility and invasion, mirror the dual relationship of CD44 expression to clinical outcome of breast cancer patients. In breast cancers, over-expression of the standard or specific variant forms have not been consistently demonstrated to relate to outcome parameters (Ma, W., Y. Deng, and L. Zhou, The prognostic value of adhesion molecule CD44v6 in women with primary breast carcinoma: a clinicopathologic study. Clin Oncol (R Coll Radiol), 2005. 17(4): p. 258-63; Agnantis, N. J., et al., Tumor markers in cancer patients. an update of their prognostic significance. Part II. In Vivo, 2004. 18(4): p. 481-8). One conclusion from these studies is that CD44 variant forms, which are expressed at different stages of breast tumor progression than the standard CD44 form (Auvinen, P., et al., Expression of CD 44 s, CD 44 v 3 and CD 44 v 6 in benign and malignant breast lesions: correlation and colocalization with hyaluronan. Histopathology, 2005. 47(4): p. 420-8; Naor, D., et al., CD44 in cancer. Crit. Rev Clin Lab Sci, 2002. 39(6): p. 527-79) perform different functions in tumor progression, and that different CD44 protein forms act as tumor suppressor during early stages of breast cancer but as enhancers of metastases during later stages (Abraham, B. K., et al., Prevalence of CD44+/CD24−/low cells in breast cancer may not be associated with clinical outcome but may favor distant metastasis. Clin Cancer Res, 2005. 11(3): p. 1154-9).

Rhamm (CD 168): Rhamm is a hyaluronan receptor that is structurally unrelated to CD44. Despite these structural dissimilarities, Rhamm performs many similar functions to CD44, among which is the ability to mediate motogenic signaling by HA. Furthermore, cell surface Rhamm can substitute for CD44 in some of its motogenic functions in vivo (34), and its high expression has consistently been linked with aggressive tumors. In particular, its over-expression in breast cancer cell subsets correlates with high expression of ERK1,2 and is an independent prognostic indicator of poor outcome and increased peripheral metastasis (Wang, C., et al., The overexpression of RHAMM, a hyaluronan-binding protein that regulates ras signaling, correlates with overexpression of mitogen-activated protein kinase and is a significant parameter in breast cancer progression. Clin Cancer Res, 1998. 4(3): p. 567-76). Unlike CD44, cell surface Rhamm is not an integral protein but soluble forms of this protein bind to the cell surface via previously unidentified cell surface co-receptors. Rhamm also occurs in intracellular compartments and some of the functions of these intracellular Rhamm isoforms may be distinct from its cell surface counterparts in motogenic signaling (Turley, E. A., P. W. Noble, and L. Y. Bourguignon, Signaling properties of hyaluronan receptors. J Biol Chem, 2002. 277(7): p. 4589-92; Toole, B. P., Hyaluronan: from extracellular glue to pericellular cue. Nat Rev Cancer, 2004. 4(7): p. 528-39; Adamia, S., C. A. Maxwell, and L. M. Pilarski, Hyaluronan and hyaluronan synthases: potential therapeutic targets in cancer. Curr Drug Targets Cardiovasc Haematol Disord, 2005. 5(1): p. 3-14). However, a scenario where intracellular Rhamm forms bind to ERK1 and also to CD44 while cell surface forms of Rhamm bind to the extracellular sequence of CD44 would also be consistent with our present and previous studies. For example, previous studies have suggested that the presence of cell surface Rhamm is required for HA-mediated activation of protein tyrosine kinase cascades and of ERK1,2 in endothelial cells (Lokeshwar, V. B. and M. G. Selzer, Differences in hyaluronic acid-mediated functions and signaling in arterial, microvessel, and vein-derived human endothelial cells. J Biol Chem, 2000. 275(36): p. 27641-9) and fibroblasts (Zhang, S., et al., The hyaluronan receptor RHAMM regulates extracellular-regulated kinase. J Biol Chem, 1998. 273(18): p. 11342-8; Hamilton, S. R., et al., The hyaluronan receptors CD44 and Rhamm (CD168) form complexes with ERK1,2 that sustain high basal motility in breast cancer cells. J Biol Chem, 2007. 282(22): p. 16667-80), and for the motility and invasion of endothelial cells (Lokeshwar, V. B. and M. G. Selzer, Differences in hyaluronic acid-mediated functions and signaling in arterial, microvessel, and vein-derived human endothelial cells. J Biol Chem, 2000. 275(36): p. 27641-9) when CD44 is co-expressed. We have also demonstrated that genetic deletion of Rhamm ablates a motogenic response of dermal fibroblasts to HA even though these cells retain expression of CD44 protein (unpublished data). These previous studies, along with the data in the current study, provide the context for our conclusions that Rhamm is required to sustain motility of aggressive breast cancer cells. We also propose that Rhamm complexes with CD44 (as an integral membrane protein with conflicting prognostic value in breast cancer) and ERK1,2 to enhance basal ERK1,2 activity and sustain motogenic signaling in invasive cancer cells. Although the mechanisms responsible for these effects are not yet defined, the ability of specific protein partners, including non-integral proteins, to modify responses of integral receptors to their ligands is well documented. For example, the ability of UPA/UPAR interactions to promote mitogenesis depends upon the presence of EGFR, which complexes with UPAR and thus modifies signaling output (Jo, M., et al., Dynamic assembly of the urokinase-type plasminogen activator signaling receptor complex determines the mitogenic activity of urokinase-type plasminogen activator. J Biol Chem, 2005. 280(17): p. 17449-57).

ERK1,2: ERK1,2 are ubiquitous and highly homologous MAP kinases that mediate proliferation, differentiation and motility via growth factor and ECM receptor activation. Over-expression and elevated activation of these kinases are common in human tumors. In particular, the importance of increased ERK1,2 activity in breast cancer is demonstrated by the anti-tumor effects of a specific inhibitor (PD184352) of the upstream kinase activators MEK1,2 in breast cancer patients (Allen, L. F., J. Sebolt-Leopold, and M. B. Meyer, CI-1040 (PD184352), a targeted signal transduction inhibitor of MEK (MAPKK). Semin Oncol, 2003. 30(5 Suppl 16): p. 105-16). Those anti-tumor effects correlate with inhibition of ERK1,2 activation (Ibid). However, reports differ as to the usefulness of ERK1,2 expression or activity as a prognostic factor in this neoplastic disease (e.g. (Wang, Z., et al., Expression of extracellular signal-regulated kinase and its relationship with clinicopathological characteristics of breast cancer. Zhonghua Zhong Liu Za Zhi, 2002. 24(4): p. 360-3; Milde-Langosch, K., et al., Expression and prognostic relevance of activated extracellular-regulated kinases (ERK1/2) in breast cancer. Br J Cancer, 2005. 92(12): p. 2206-15; Hagan, S., et al., Reduction of Raf-1 kinase inhibitor protein expression correlates with breast cancer metastasis. Clin Cancer Res, 2005. 11(20): p. 7392-7)]. The discrepancy between the effectiveness of anti-ERK1,2 therapy in breast cancer and variable usefulness as a prognostic factor may result from the complex manner in which ERK1,2 activity regulates specific and often opposing cell functions (i.e. differentiation vs motility). MAP kinases are among the most common effectors in growth factor and ECM-regulated signaling pathways, but a variety of temporal, spatial and quantitative cues determine whether or not activation of these MAP kinases results, for example, in proliferation or motility/invasion (Kuida, K. and D. M. Boucher, Functions of MAP kinases: insights from gene-targeting studies. J Biochem (Tokyo), 2004. 135(6): p. 653-6; Boldt, S, and W. Kolch, Targeting MAPK signalling: Prometheus' fire or Pandora's box? Curr Pharm Des, 2004. 10(16): p. 1885-905). Thus, sustained activation of ERK1,2 is required to initiate motility of breast cancer cells (Boldt, S, and W. Kolch, Targeting MAPK signalling: Prometheus' fire or Pandora's box? Curr Pharm Des, 2004. 10(16): p. 1885-905).

ERK1,2 activity is determined by many factors including concentration of the stimulus, receptor dimerization, presence of co-receptors that modify the rate of receptor internalization, and intracellular scaffolding/accessory proteins (Kuida, K. and D. M. Boucher, Functions of MAP kinases: insights from gene-targeting studies. J Biochem (Tokyo), 2004. 135(6): p. 653-6; Boldt, S, and W. Kolch, Targeting MAPK signalling: Prometheus' fire or Pandora's box? Curr Pharm Des, 2004. 10(16): p. 1885-905). The mechanisms by which CD44 and Rhamm sustain high basal ERK1,2 activity remains unclear but confocal analysis shows that these HA receptors colocalize with phospho-ERK1,2, predominantly in the perinuclear area of cells where these proteins appear as vesicles. Although internalization of some receptors can inhibit ERK1,2 activity, internalization of others (i.e. Protease-activated Receptor-2 [PAR-2]) sustains ERK1,2 activation (Ge, L., et al., Constitutive protease-activated receptor-2-mediated migration of MDA MB-231 breast cancer cells requires both beta-arrestin-1 and -2. J Biol Chem, 2004. 279(53): p. 55419-24). Since both CD44 and Rhamm associate with ERK1,2 (Zhang, S., et al., The hyaluronan receptor RHAMM regulates extracellular-regulated kinase. J Biol Chem, 1998. 273(18): p. 11342-8; Bourguignon, L. Y., et al., Hyaluronan-CD44 interaction with IQGAP1 promotes Cdc42 and ERK signaling, leading to actin binding, Elk-1/estrogen receptor transcriptional activation, and ovarian cancer progression. J Biol Chem, 2005. 280(12): p. 11961-72), we propose that internalization of these receptors and their trafficking to the perinuclear area as vesicles constitute either recycling endosomes containing key adhesion proteins required for cycles of attachment and detachment during motility and/or a specific class of “signalosome” that could promote sustained ERK1,2 activity in the cytoplasm. By this type of mechanism, active ERK1,2 would therefore be available to traffic to key cytoplasmic compartments such as focal adhesions or the nucleus, sites where ERK1,2 activity is known to be required for cell motility/invasion (Reddy, K. B., S. M. Nabha, and N. Atanaskova, Role of MAP kinase in tumor progression and invasion. Cancer Metastasis Rev, 2003. 22(4): p. 395-403; Viala, E. and J. Pouyssegur, Regulation of tumor cell motility by ERK mitogen-activated protein kinases. Ann N Y Acad Sci, 2004. 1030: p. 208-18).

The term “MDA-MB-231” refers to an aggressive breast cancer cell line that expresses a mutant K-Ras and activated H-Ras and that exhibit progenitor characteristics in that these cells can differentiate into ductal epithelium and express high levels of CD44/Rhamm and low levels of CD24.

The term “MCF-7” refers to a breast cell line that does not harbour any Ras mutations and does not exhibit the progenitor phenotype as herein described.

The term “MCF10A” refers to an immortalized normal breast epithelial cell line.

The term “antibody” refers to an immunoglobulin, which specifically binds to and is thereby defined as complementary with a particular spatial and polar organization of another molecule. The antibody can be monoclonal or polyclonal and can be prepared by techniques that are well known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab[prime])2, Fab[prime], and the like. In addition, aggregates, polymers, and conjugates of immunoglobulins or their fragments can be used where appropriate so long as binding affinity for a particular molecule is maintained.

A “label” or “detectable label” is a composition detectable by spectroscopic, photochemical, biochemical, immunochemical, or chemical means. For example, useful labels include radioisotopes (e.g., ³H, ³⁵S, ³²P, ⁵¹Cr, or ¹²⁵I), fluorescent dyes, electron-dense reagents, enzymes (e.g., alkaline phosphatase, horseradish peroxidase, or others commonly used in an ELISA), biotin, digoxigenin, or haptens and proteins for which antisera or monoclonal antibodies are available (e.g., the polypeptide encoded by SEQ ID NO: 5 can be made detectable, e.g., by incorporating a radiolabel into the peptide, and used to detect antibodies specifically reactive with the peptide).

The terms “semiconductor nanocrystal,” “quantum dot,” and “qdot” are used herein to refer to luminescent semiconductor nanocrystals, i.e., nanoparticles comprising a core and a shell and capable of emitting electromagnetic radiation (i.e., a signal) upon excitation by an energy source.

### IV. Interaction and Presence of Rhamm/CD44 Complexes

Since HA-binding by CD44 is associated with tumor cell motility and invasion, we hypothesized the possibility that co-expression of other HA-binding proteins (i.e., Rhamm) could modify the effects of high CD44 expression on breast tumor cell behavior. In the Examples, we showed that invasive breast cancer cells (MDA-MB-231 and Ras-MCF10A) sustain high levels of ERK1,2 activation upon growth factor/motogenic stimulation when cell surface CD44 and Rhamm are co-expressed. By contrast, less invasive lines (MCF7 and MCF10A) have predominant expression of only one of these HA receptors and only transiently activate ERK1,2 in response to growth factor stimulation. We have demonstrated that CD44 and Rhamm co-associate with ERK1,2 in complexes and that formation of these complexes correlate with invasiveness. Finally, we showed that CD44/Rhamm/ERK1,2 complexes are required for basal motility of the more invasive cell lines but are not involved in basal motility of the less invasive cell lines. The results are consistent with a model in which CD44, Rhamm and activated ERK1,2 (linked physically and functionally) contribute to motility, invasion, and malignant progression in breast cancer.

We identified an autocrine motility mechanism by which aggressive breast cancer cell lines such as MDA-MB-231 and Ras-MCF10A cells maintain high basal rates of motility. This mechanism requires HA production, ERK1,2 activity, and the HA receptors CD44 and Rhamm (CD168) and is associated with the formation of signaling complexes composed of cell surface CD44, Rhamm, and active ERK1,2 that are exclusive to the aggressive, highly motile breast cancer cell lines. Although previous reports have demonstrated that either CD44 or cell surface Rhamm are required for HA-mediated motility of tumor cells including MDA-MB-231 cells, this is the first report documenting both a functional and physical interaction between these two HA receptors and demonstrating the coupling of this complex to a motogenic signaling pathway through ERK1,2. Previous reports have noted that CD44 interacts with ERK2 via IQGAP1 (Bourguignon, L. Y., et al., Hyaluronan-CD44 interaction with IQGAP1 promotes Cdc42 and ERK signaling, leading to actin binding, Elk-1/estrogen receptor transcriptional activation, and ovarian cancer progression. J Biol Chem, 2005. 280(12): p. 11961-72.) while Rhamm (likely intracellular forms) associates with ERK1 Z(hang, S., et al., The hyaluronan receptor RHAMM regulates extracellular-regulated kinase. J Biol Chem, 1998. 273(18): p. 11342-8; Tolg, C., et al., Rhamm−/− fibroblasts are defective in CD44-mediated ERK1,2 motogenic signaling, leading to defective skin wound repair. J Cell Biol, 2006. 175(6): p. 1017-28). Therefore, our results imply that one function for the formation of Rhamm/CD44 complexes is to compartmentalize and link both MAP kinases to an HA motogenic pathway. An increasing number of reports indicate that ERK1 and ERK2 perform distinct functions during cellular processes such as proliferation and differentiation (Lips, D. J., et al., MEK1-ERK2 signaling pathway protects myocardium from ischemic injury in vivo. Circulation, 2004. 109(16): p. 1938-41; Nekrasova, T., et al., ERK1-deficient mice show normal T cell effector function and are highly susceptible to experimental autoimmune encephalomyelitis. J Immunol, 2005. 175(4): p. 2374-80; Pages, G. and J. Pouyssegur, Study of MARK signaling using knockout mice. Methods Mol Biol, 2004. 250: p. 155-66), which raise the possibilities that these MAP kinases also perform distinct functions during motility (Providence, K. M. and P. J. Higgins, PAI-1 expression is required for epithelial cell migration in two distinct phases of in vitro wound repair. J Cell Physiol, 2004. 200(2): p. 297-308) and that both are required for sustaining high basal motility rates in transformed cells. Our results indicate that the association of Rhamm with CD44 and the subsequent association of this complex with ERK1,2 may modify tumor suppression by CD44 to favor latent tumor promoter functions, resulting in an increased tendency of breast tumor cells to metastasize during clinical progression. This is consistent with the strong association amongst elevated HA accumulation, ERK1,2 activity and Rhamm expression with aggressive forms of breast carcinoma (Liu, R., et al., The prognostic role of a gene signature from tumorigenic breast-cancer cells. N Engl J Med, 2007. 356(3): p. 217-26).

Increased expression of full length Rhamm (85 kDa isoform) was shown in both MDA-MB-231 and Ras-MCF10A cells compared to MCF7 and parental MCF10A cells, consistent with previous reports that full-length Rhamm is not expressed (or is expressed at very low levels) in normal cells or tissues. Also, shorter Rhamm isoforms (i.e. 63 and 43 kDa) and CD44s levels were increased in both MDA-MB-231 and Ras-MCF10A cells. It is currently unclear how these shorter Rhamm isoforms are generated, although they have been observed in a number of human cancer cell lines, including breast cancer and melanoma cell lines (Hofmann, M., et al., Identification of IHABP, a 95 kDa intracellular hyaluronate binding protein. J Cell Sci, 1998. 111 (Pt 12): p. 1673-84; Ahrens, T., et al., CD44 is the principal mediator of hyaluronic-acid-induced melanoma cell proliferation. J Invest Dermatol, 2001. 116(1): p. 93-101), and a protein form similar to the 63 kD can be transforming in murine cells. The apparently preferential association of the 63 kD isoform with ERK1,2, raises the possibility that each isoform has distinct binding functions. Differences in protein interaction of the Rhamm isoforms could be a major contributing factor to Rhamm mediated constitutive activation of the ERK1,2 pathway.

Constitutive activation of the ERK1,2 pathway is associated with both poor outcome in breast cancer [100] and with a progenitor (basal cell) phenotype [Laakso, M., et al., Cytokeratin 5/14-positive breast cancer: true basal phenotype confined to BRCA1 tumors. Mod Pathol, 2005. 18(10): p. 1321-8; Tanner, M., et al., Characterization of a novel cell line established from a patient with Herceptin-resistant breast cancer. Mol Cancer Ther, 2004. 3(12): p. 1585-92]. Furthermore, Rhamm protein levels in tumor subsets correlate with ERK1,2 protein levels. Collectively these results link CD44, Rhamm and ERK1,2 to breast tumor progenitor cells and to poor outcome associated with aggressive neoplastic disease.

These results provide that rapid HA uptake is a marker for detecting a breast tumor progenitor phenotype. Furthermore, our data show that we can use labeled hyaluronan in culture and in vivo to identify highly tumorigenic cell populations that exhibit an aggressive phenotype characterized by high Rhamm and CD44 expression.

### V. Identifying Cancer Progenitor Cells

In one embodiment, the present invention provides a method that permits identification of whether or not tumor (e.g., breast tumor) biopsies contain highly tumorigenic progenitor subsets that can put the cancer patient at risk for developing metastases. The method comprises providing a sample and detecting the presence of CD44/Rhamm complexes. Where the sample contains the CD44/Rhamm complexes, this indicates that the sample contains cancer progenitor cells. In another embodiment, a method for identifying cancer progenitor cells in a patient is described herein. By identifying cancer progenitor cells, the present method allows for the development of novel therapeutics to selectively kill or force terminal differentiation of these progenitor cells. For example, this technology can be used for in vivo imaging of tumors that contain highly tumorigenic progenitor cell subsets which allows for selective treatment of patients at risk for metastasis vs. those not at risk. Most importantly, this technology can be used to deliver anti-cancer drugs to the highly tumorigenic progenitor subsets present in primary breast cancers. It is important to note also that although hyaluronan can be used as the delivery vehicle/ligand, so could antibodies to CD44 or RHAMM as well as mimetics of HA including peptide or small molecule mimetics as well as peptide or small molecule mimetics of either CD44 or Rhamm.

In another embodiment, a method to determine the potential efficacy of using HA-metal nanoparticles to image highly tumorigenic cells with this phenotype in vivo with the longer-term goal of utilizing the present methods to better image and diagnose tumours in patients. Subsets of the highly tumorgenic CD44⁺/CD24^{-/low}/lineage⁻/ESA⁺ breast tumor cells should also selectively express high levels of Rhamm protein and that as a consequence of this surface phenotype, these tumor cells subsets will likely internalize HA more rapidly than surrounding normal/less tumorigenic cells. Therefore, the use of HA-metal conjugates should permit detection of these highly tumorigenic subsets.

Another embodiment of the invention provides a method for prognosing cancer. The method contemplates a use for assessing malignant progenitors correlated to patient outcome, and allows clinicians to sort patients with primary cancers and identify those patients at risk for metastases and those patients whose cancer is not or less likely to metastasize. In turn this sorting permits appropriate tailoring of therapeutic intervention for each patient based upon their risk for metastases. This is an important development in the art, as currently breast cancer patients that present with tumors are often channeled through the same therapeutic regime as currently the best indicator of poor outcome is the very crude measure of primary tumor size.

Thus, one embodiment of the invention provides that the methods for prognosing cancer comprises providing a cancer cell and detecting the presence or absence of CD44/Rhamm complexes, whereby the presence of the CD44/Rhamm complexes indicates that an aggressively metastatic cancer cell.

The sample provided is typically a cell, tissue sample or biopsy from a patient suspected of having breast cancer, colon cancer, gastric cancer, gliomas, and other parenchymal tumors; leukemias, multiple myeloma and other immune-cell related tumors; desmoid and other mesenchymal related tumors and skin cancers such as basal cell carcinoma and melanoma. In one embodiment, the cell detected is a breast cancer cell.

In another embodiment, the methods for prognosing cancer are performed in vivo by imaging the CD44/Rhamm complexes in a subject. Thus, the present invention also described compositions and methods for imaging CD44/Rhamm complexes to image tumorigenic cell populations in vivo in a subject.

In a preferred embodiment, detection of CD44/Rhamm complexes is carried out by a probe, comprising a targeting component and an imaging component. In one embodiment, the presence of CD44/Rhamm complexes is detected by contacting the sample with a probe that specifically binds to the CD44/Rhamm complex. Optionally, this probe may be labeled with a detectable marker to allow detection of the location of the cancer progenitor cell. Further, the detectable label allows the movement and development of the progenitor cell subsets.

Methods of preparing probes are well known to those of skill in the art (see, e.g. Sambrook et al., Molecular Cloning: A Laboratory Manual (2nd ed.), Vols. 1-3, Cold Spring Harbor Laboratory, (1989) or Current Protocols in Molecular Biology, F. Ausubel et al., ed. Greene Publishing and Wiley-Interscience, New York (1987)), which are hereby incorporated by reference.

Targeting Component. The CD44/Rhamm complex targeting component may be a small molecule, monoclonal or polyclonal antibodies, recombinant proteins and other expression products, antisense oligonucleotides, siRNA, aptamers, peptides or peptidomimetics.

Small molecule compounds. In one embodiment, the targeting component is a small molecule, preferably of MW of 200-800 Daltons. In one embodiment, high throughput screening (HTS) methods are used to identify small molecule compounds that target CD44/Rhamm complexes. HTS methods involve providing a combinatorial chemical or peptide library containing a large number of potential therapeutic compounds (i.e., compounds that target CD44/Rhamm complexes). Such “libraries” are then screened in one or more assays, as described herein, to identify those library members (particular peptides, chemical species or subclasses) that display the desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, 5,288,514, and the like).

Devices for the preparation of combinatorial libraries are commercially available (see, e.g., ECIS TM, Applied BioPhysics Inc., Troy, N.Y., MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

Antibodies. In another embodiment, the targeting component is an antibody. In this embodiment, the presence of CD44/Rhamm complexes is detected by contacting the sample with an antibody that specifically binds to the CD44/Rhamm complex. Optionally, this antibody may be labeled with a detectable marker to allow detection of the location of the cancer progenitor cell. Further, the detectable label allows the movement and development of the progenitor cell subsets.

For preparation of antibodies, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3.sup.rd ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The phrase “specifically binds to, or “selectively binds to” or “specifically immunoreactive with,” or “selectively binds to” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies raised to a Rhamm protein, polymorphic variants, alleles, orthologs, and conservatively modified variants, or splice variants, or portions thereof, can be selected to obtain only those polyclonal antibodies that are specifically immunoreactive with Rhamm proteins and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

Rhamm specific antibodies can be made by a number of methods known in the art. In one embodiment, specific Rhamm antibodies are generated by first amplifying and cloning cDNA fragments of SEQ ID NOS: 1 or 3. A cDNA sequence such as SEQ ID NO: 1 is amplified and cloned, and then expressed peptide fragments of Rhamm from the cloned cDNAs are obtained.

In another embodiment, peptide fragments are synthesized to generate peptide fragments. These peptide fragments may include portions of the Rhamm 60 aa isoform insertion and may contain the adjacent Rhamm amino acid sequence.

Since synthesized peptides are not always immunogenic on their own, the peptides are conjugated to a carrier protein before use. Appropriate carrier proteins include, but are not limited to, Keyhole limpet hemacyanin (KLH), bovine serum albumin (BSA) and ovalbumin (OVA). The conjugated peptides should then be mixed with adjuvant and injected into a mammal, preferably a rabbit through intradermal injection, to elicit an immunogenic response. Samples of serum can be collected and tested by ELISA assay to determine the titer of the antibodies and then harvested.

Polyclonal antibodies can be purified by passing the harvested antibodies through an affinity column. However, monoclonal antibodies are preferred over polyclonal antibodies and can be generated according to standard methods known in the art of creating an immortal cell line which expresses the antibody.

Nonhuman antibodies are highly immunogenic in human thus limiting their therapeutic potential. In order to reduce their immunogenicity, nonhuman antibodies need to be humanized for therapeutic application. Through the years, many researchers have developed different strategies to humanize the nonhuman antibodies. One such example is using “HuMAb-Mouse” technology available from MEDAREX, Inc. (Princeton, N.J.). “HuMAb-Mouse” is a strain of transgenic mice that harbors the entire human immunoglobin (Ig) loci and thus can be used to produce fully human monoclonal Rhamm antibodies.

Immunoblotting using the specific antibodies of the invention with a control sequence should not produce a detectable signal at preferably 0.5-10 fold molar excess (relative to the Rhamm detection), more preferably at 50 fold molar excess and most preferably no signal is detected at even 100 fold molar excess.

Hall, C L, Wang FS and Turley E A, Src−/− fibroblasts are defective in their ability to disassemble focal adhesions in response to phorbol ester/hyaluronan treatment, Cell Commun Adhes 2002 September-December; 9(5-6):273-83 describe the preparation of antibodies, which may find use as a targeting component in the present application.

Antisense nucleic acids, aptamers and siRNA. In another embodiment, the targeting component is an antisense nucleic acid or oligonucleotide. Such antisense oligonucleotides may include but are not limited to, siRNA oligonucleotides, antisense oligonucleotides, peptide inhibitors and aptamer sequences that bind to CD44/Rhamm complexes. “RNAi molecule” or an “siRNA” refers to a nucleic acid that forms a double stranded RNA, which double stranded RNA has the ability to reduce or inhibit expression of a gene or target gene when the siRNA expressed in the same cell as the gene or target gene. “siRNA” thus refers to the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is 15-50 nucleotides in length, and the double stranded siRNA is about 15-50 base pairs in length, preferable about preferably about 20-30 base nucleotides, preferably about 20-25 nucleotides in length, e.g., 20, 21, 22, 23, 24, 25, 26, 27, 28, 29, or 30 nucleotides in length.

In one embodiment, RNA interference is used to generate small double-stranded RNA (small interference RNA or siRNA) inhibitors to affect the expression of a candidate gene generally through cleaving and destroying its cognate RNA. Small interference RNA (siRNA) is typically 19-22 nt double-stranded RNA. siRNA can be obtained by chemical synthesis or by DNA-vector based RNAi technology. Using DNA vector based siRNA technology, a small DNA insert (about 70 bp) encoding a short hairpin RNA targeting the gene of interest is cloned into a commercially available vector. The insert-containing vector can be transfected into the cell, and expressing the short hairpin RNA. The hairpin RNA is rapidly processed by the cellular machinery into 19-22 nt double stranded RNA (siRNA). In a preferred embodiment, the siRNA is inserted into a suitable RNAi vector because siRNA made synthetically tends to be less stable and not as effective in transfection.

siRNA can be made using methods and algorithms such as those described by Wang L, Mu F Y. (2004) A Web-based Design Center for Vector-based siRNA and siRNA cassette. Bioinformatics. (In press); Khvorova A, Reynolds A, Jayasena S D. (2003) Functional siRNAs and miRNAs exhibit strand bias. Cell. 115(2):209-16; Harborth J, Elbashir S M, Vandenburgh K, Manninga H, Scaringe S A, Weber K, Tuschl T. (2003) Sequence, chemical, and structural variation of small interfering RNAs and short hairpin RNAs and the effect on mammalian gene silencing. Antisense Nucleic Acid Drug Dev. 13(2):83-105; Reynolds A, Leake D, Boese Q, Scaringe S, Marshall W S, Khvorova A. (2004) Rational siRNA design for RNA interference. Nat. Biotechnol. 22(3):326-30 and Ui-Tei K, Naito Y, Takahashi F, Haraguchi T, Ohki-Hamazaki H, Juni A, Ueda R, Saigo K. (2004) Guidelines for the selection of highly effective siRNA sequences for mammalian and chick RNA interference. Nucleic Acids Res. 32(3):936-48, which are hereby incorporated by reference.

Other tools for constructing siRNA sequences are web tools such as the siRNA Target Finder and Construct Builder available from GenScript (http://www.genscript.com), Oligo Design and Analysis Tools from Integrated DNA Technologies (URL:<http://www.idtdna.com/SciTools/SciTools.aspx>), or siDESIGN™ Center from Dharmacon, Inc. (URL:<http://design.dharmacon.com/default.aspx?source=0>). siRNA are suggested to built using the ORF (open reading frame) as the target selecting region, preferably 50-100 nt downstream of the start codon. Because siRNAs function at the mRNA level, not at the protein level, to design an siRNA, the precise target mRNA nucleotide sequence may be required. Due to the degenerate nature of the genetic code and codon bias, it is difficult to accurately predict the correct nucleotide sequence from the peptide sequence. Additionally, since the function of siRNAs is to cleave mRNA sequences, it is important to use the mRNA nucleotide sequence and not the genomic sequence for siRNA design, although as noted in the Examples, the genomic sequence can be successfully used for siRNA design. However, designs using genomic information might inadvertently target introns and as a result the siRNA would not be functional for silencing the corresponding mRNA.

Rational siRNA design should also minimize off-target effects which often arise from partial complementarity of the sense or antisense strands to an unintended target. These effects are known to have a concentration dependence and one way to minimize off-target effects is often by reducing siRNA concentrations. Another way to minimize such off-target effects is to screen the siRNA for target specificity.

In one embodiment, the siRNA can be modified on the 5′-end of the sense strand to present compounds such as fluorescent dyes, chemical groups, or polar groups. Modification at the 5′-end of the antisense strand has been shown to interfere with siRNA silencing activity and therefore this position is not recommended for modification. Modifications at the other three termini have been shown to have minimal to no effect on silencing activity.

It is recommended that primers be designed to bracket one of the siRNA cleavage sites as this will help eliminate possible bias in the data (i.e., one of the primers should be upstream of the cleavage site, the other should be downstream of the cleavage site). Bias may be introduced into the experiment if the PCR amplifies either 5′ or 3′ of a cleavage site, in part because it is difficult to anticipate how long the cleaved mRNA product may persist prior to being degraded. If the amplified region contains the cleavage site, then no amplification can occur if the siRNA has performed its function.

In another embodiment, antisense oligonucleotides (“oligos”) can be designed. Antisense oligonucleotides are short single-stranded nucleic acids, which function by selectively hybridizing to their target mRNA, thereby blocking translation. Translation is inhibited by either RNase H nuclease activity at the DNA:RNA duplex, or by inhibiting ribosome progression, thereby inhibiting protein synthesis. This results in discontinued synthesis and subsequent loss of function of the protein for which the target mRNA encodes.

In a preferred embodiment, antisense oligos are phosphorothioated upon synthesis and purification, and are usually 18-22 bases in length. It is contemplated that the PVT1 and other candidate gene antisense oligos may have other modifications such as 2′-O-Methyl RNA, methylphosphonates, chimeric oligos, modified bases and many others modifications, including fluorescent oligos.

In a preferred embodiment, active antisense oligos should be compared against control oligos that have the same general chemistry, base composition, and length as the antisense oligo. These can include inverse sequences, scrambled sequences, and sense sequences. The inverse and scrambled are recommended because they have the same base composition, thus same molecular weight and Tm as the active antisense oligonucleotides. Rational antisense oligo design should consider, for example, that the antisense oligos do not anneal to an unintended mRNA or do not contain motifs known to invoke immunostimulatory responses such as four contiguous G residues, palindromes of 6 or more bases and CG motifs.

Antisense oligonucleotides can be used in vitro in most cell types with good results. However, some cell types require the use of transfection reagents to effect efficient transport into cellular interiors. It is recommended that optimization experiments be performed by using differing final oligonucleotide concentrations in the 1-5 μm range with in most cases the addition of transfection reagents. The window of opportunity, i.e., that concentration where you will obtain a reproducible antisense effect, may be quite narrow, where above that range you may experience confusing non-specific, non-antisense effects, and below that range you may not see any results at all. In a preferred embodiment, down regulation of the targeted mRNA (e.g. Rhamm cDNA SEQ ID NO: 1) will be demonstrated by use of techniques such as northern blot, real-time PCR, cDNA/oligo array or western blot. The same endpoints can be made for in vivo experiments, while also assessing behavioral endpoints.

For cell culture, antisense oligonucleotides should be re-suspended in sterile nuclease-free water (the use of DEPC-treated water is not recommended). Antisense oligonucleotides can be purified, lyophilized, and ready for use upon re-suspension. Upon suspension, antisense oligonucleotide stock solutions may be frozen at −20° C. and stable for several weeks.

In another embodiment, aptamer sequences which bind to specific RNA or DNA sequences can be made. Aptamers are synthetic oligonucleotides designed and selected to bind a certain target with high affinity and sensitivity, based upon unique folding and tertiary structure. Aptamers can be used as alternative candidates to antibodies in the present methods. As used herein, the terms “aptamer(s)” or “aptamer sequence(s)” are meant to refer to single stranded nucleic acids (RNA or DNA) whose distinct nucleotide sequence determines the folding of the molecule into a unique three dimensional structure. Aptamers comprising 15 to 120 nucleotides can be selected in vitro from a randomized pool of oligonucleotides (10¹⁴-10¹⁵ molecules). The “aptamers or aptamer sequences” comprise a degenerate sequence, and can further comprise fixed sequences flanking the degenerate sequence. The term “aptamer” as used herein further contemplates the use of both native and modified DNA and RNA bases, e.g. beta-D-Glucosyl-Hydroxymethyluracil.

Nucleic acids are easily synthesized or amplified by PCR; therefore a vast supply of consistent quality is available. Also, nucleic acids can easily be modified to incorporate tags, such as biotin or fluorescent molecules, for detection and/or immobilization. Additionally, aptamers are smaller (<25 kDa) and more stable than antibodies. Moreover, unlike the requirement of milligram quantities of protein or peptide for antibody production, only microgram quantities of protein or peptide are required for aptamer SELEX.

The idea of using single stranded nucleic acids (aptamers) as affinity molecules for proteins has shown modest progress. See Tuerk C, Gold L. (1990) Systematic evolution of ligands by exponential enrichment: RNA ligands to bacteriophage T4 DNA polymerase. Science. August 3; 249(4968):505-10; Ellington A D, Szostak J W. (1990) In vitro selection of RNA molecules that bind specific ligands. Nature. August 30; 346(6287):818-22; and Ellington A D, Szostak J W. (1992) Selection in vitro of single-stranded DNA molecules that fold into specific ligand-binding structures. Nature. February 27; 355(6363):850-2. The concept is based on the ability of short oligomer (20-80 mer) sequences to fold, in the presence of a target, into unique 3-dimensional structures that bind the target with high affinity and specificity. Aptamers are generated by a process that combines combinatorial chemistry with in vitro evolution, commonly known as SELEX (Systematic Evolution of Ligands by Exponential Enrichment). Following the incubation of a protein with a library of DNA or RNA sequences (typically 10¹⁴ molecules in complexity) protein-DNA complexes are isolated, the DNA is amplified, and the process is repeated until the sample is enriched with sequences that display high affinity for the protein of interest. Since the selection pressure is high affinity for the target, aptamers with low nanomolar affinities may be obtained. Aptamers offer advantages over protein-based affinity reagents because nucleic acids possess increased stability, ease of regeneration (PCR or oligonucleotide synthesis), and simple modification for detection and immobilization.

Aptamer sequences can be isolated through methods such as those disclosed in co-pending U.S. patent application Ser. No. 10/934,856 (published as U.S. Patent Publication No. 20050142582), which is hereby incorporated by reference.

Recombinant expression products. In yet another embodiment, the targeting component is a recombinant protein. Substantially identical nucleic acids encoding sequences of Rhamm inhibitors can be isolated using nucleic acid probes and oligonucleotides under stringent hybridization conditions, by screening libraries. Alternatively, expression libraries can be used to clone these sequences, by detecting expressed homologues immunologically with antisera or purified antibodies made against the core domain of nucleic acids encoding Rhamm inhibitor sequences. See Yang, B., et al., Identification of a common hyaluronan binding motif in the hyaluronan binding proteins RHAMM, CD44 and link protein. Embo J, 1994. 13(2): p. 286-96 hereby incorporated by reference.

Gene expression of RHAMM and CD44 can also be analyzed by techniques known in the art, e.g., reverse transcription and amplification of mRNA, isolation of total RNA or poly A+ RNA, northern blotting, dot blotting, in situ hybridization, RNase protection, probing DNA microchip arrays, and the like.

To obtain high level expression of a cloned gene or nucleic acid sequence, such as those cDNAs encoding nucleic acid sequences encoding CD44 and Rhamm recombinant proteins, CD44 and/or Rhamm inhibitors such as CD44 and Rhamm cDNAs or an siRNA targeting Rhamm and related nucleic acid sequence homologues, one typically subclones a sequence (e.g., nucleic acid sequences encoding CD44 and Rhamm recombinant proteins) into an expression vector that is subsequently transfected into a suitable host cell. The expression vector typically contains a strong promoter or a promoter/enhancer to direct transcription, a transcription/translation terminator, and for a nucleic acid encoding a protein, a ribosome binding site for translational initiation. The promoter is operably linked to the nucleic acid sequence encoding CD44 or Rhamm protein such as a CD44 or Rhamm cDNA or a subsequence thereof. Suitable bacterial promoters are well known in the art and described, e.g., in Sambrook et al. and Ausubel et al. The elements that are typically included in expression vectors also include a replicon that functions in a suitable host cell such as E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to the recombinant CD44 or RHAMM recombinant expression products (e.g., proteins, inhibitors or peptides) to provide convenient methods of isolation, e.g., His tags. In some case, enzymatic cleavage sequences (e.g., Met-(His)g-Ile-Glu-GLy-Arg which form the Factor Xa cleavage site) are added to the recombinant CD44 or RHAMM recombinant expression products. Bacterial expression systems for expressing the recombinant CD44 or RHAMM recombinant expression products and nucleic acids are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available.

Standard transfection methods are used to produce cell lines that express large quantities of a Rhamm inhibitor, which can then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of cells is performed according to standard techniques (see, e.g., Morrison, J. Bact. 132:349-351 (1977); Clark-Curtiss & Curtiss, Methods in Enzymology 101:347-362 (Wu et al., eds, 1983). For example, any of the well known procedures for introducing foreign nucleotide sequences into host cells may be used. These include the use of calcium phosphate transfection, lipofectamine, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing RHAMM inhibitor peptides and nucleic acids.

After the expression vector is introduced into the cells, the transfected cells are cultured under conditions favoring expression of recombinant CD44 or RHAMM recombinant expression products such as a recombinant CD44 or RHAMM recombinant proteins, peptides and related nucleic acid sequence homologues.

Peptides and peptidomimetics. In another embodiment, the targeting component is a peptide or peptidomimetic. In a preferred embodiment, the peptide or peptidomimetic is hyaluronan (HA), which is a ligand for these complexes, hyaluronan fragments, hyaluronan peptide or small chemical mimetics, Rhamm peptide mimetics or small chemical mimetics, and CD44 peptide mimetics or small chemical mimetics.

In another embodiment, a hyaluronan binding peptide is used to target CD44/Rhamm complexes. In a preferred embodiment, a peptide such as the peptides described by one of the inventors previously in Turley, U.S. Pat. No. 6,271,344, which is hereby incorporated by reference, is used. The sequence of a preferred hyaluronan binding peptide is substantially identical to SEQ ID NO: 5, STMMRSHKTRSHHV, and is similar to the HA binding region of Rhamm in that both have coiled coil secondary structure and similar spacing of key binding basic amino acids (BX7B “motifs”). The residues that are bolded and underlined above in SEQ ID NO:5 are responsible for Rhamm binding to HA.

The Rhamm HA binding domain sequence is KIKHVVKLK (SEQ ID NO: 6). The underlined and bolded residues show the residues in the peptide which bind to hyaluronan and mimic the Rhamm sequence. The phage peptide is expected to compete with Rhamm for hyaluronan and is essentially a Rhamm peptide mimetic.

In other embodiments, polypeptides which mimic Rhamm co-factors and Rhamm mimetics, are used to target CD44/Rhamm complexes. In one embodiment, such peptides can be made or designed based on Rhamm-binding protein sequences and functional portions of those sequences.

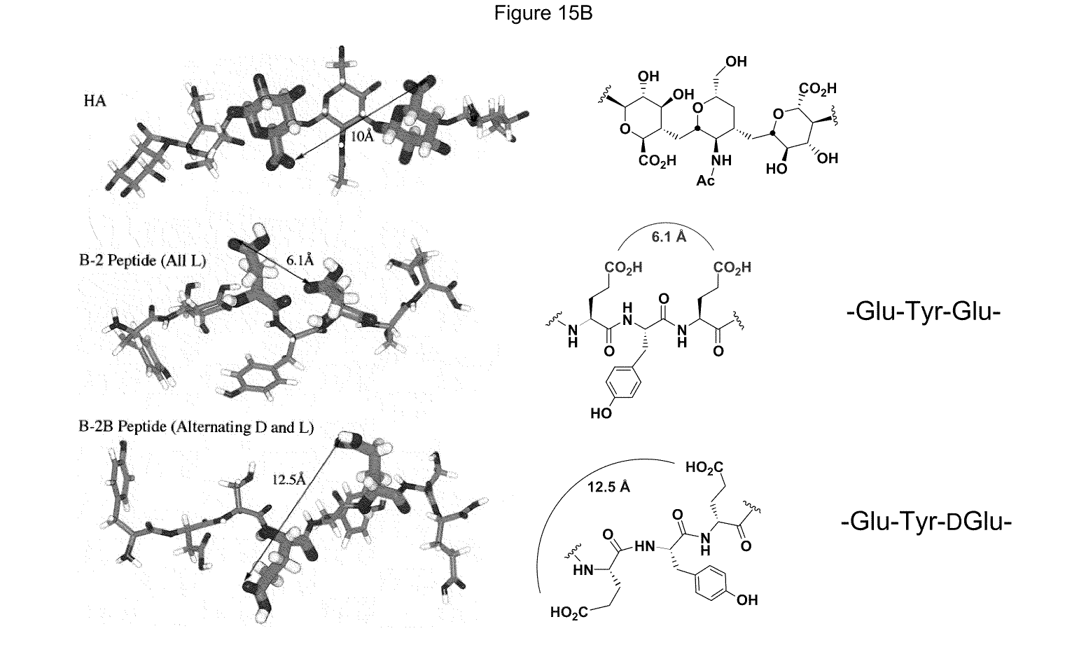

In another embodiment, a hyaluronan mimicking peptide is used to target CD44/Rhamm complexes. The sequence of a preferred hyaluronan mimicking peptide is substantially identical to the following peptides: YDSEYESE (SEQ ID NO: 8), YDSeYeSe (SEQ ID NO: 9) and YDSEYeSE (SEQ ID NO:10), GCU-NAG (hyaluronic acid), and is similar to HA such that the HA-binding region of Rhamm will recognize the polypeptide and bind to it. In another embodiment, an HA-binding peptide, whose structure is shown in FIG. 15A, is used. The HA binding peptide was isolated from a random and biased 8-mer peptide libraries screened for receptor affinity and is exhibits a Rhamm affinity of approximately 8 nM (KD). The peptide is also further described in Ziebell M R, Zhao Z-G, Luo B, Luo Y, Turley E A, and Prestwich G D. 2001. Peptides that mimic glycosaminoglycans: high-affinity ligands for a hyaluronan binding domain. Chemistry and Biology v 8: 1081-1094, hereby incorporated by reference. The peptide also exhibits competitive displacement by HA at 1 mg/mL conc.

Such a peptide is expected target CD44/Rhamm complexes and target highly tumorigenic progenitor cell populations in vivo. Structures shown in FIG. 15B can aid one having skill the art in designing HA mimetics and HA-binding peptides. Methods for designing, making and preparing HA mimetics are also described in Ziebell, M R and Prestwich G D, 2004, Interaction of Peptide Mimics of Hyaluronic Acid with the Receptor of Hyaluronan Mediated Motility (Rhamm), J. of Computer Aided Molecular Design, v 18, 597-614; and Ziebell M R, Zhao Z-G, Luo B, Luo Y, Turley E A, and Prestwich G D. 2001. Peptides that mimic glycosaminoglycans: high-affinity ligands for a hyaluronan binding domain. Chemistry and Biology v 8: 1081-1094], the teachings of both of which are hereby incorporated by reference in their entirety for all purposes.

The polypeptides can be chemically synthesized using methods well known in the art including, e.g., solid phase synthesis (see, e.g., Merrifield, J. Am. Chem. Soc., 85:2149-2154 (1963) and Abelson et al., Methods in Enzymology, Volume 289: Solid-Phase Peptide Synthesis (1st ed. 1997)). Polypeptide synthesis can be performed using manual techniques or by automation. Automated synthesis can be achieved, for example, using Applied Biosystems 431A Peptide Synthesizer (Perkin Elmer). Alternatively, various fragments of the polypeptide can be chemically synthesized separately and then combined using chemical methods to produce the full length polypeptide. The sequence and mass of the polypeptides can be verified by GC mass spectroscopy.

In yet another embodiment, peptide mimetics of the polypeptides of the present invention are provided. A “peptide mimetic” or “peptidomimetic” includes any modified form of an amino acid chain, including, but not limited to, phosphorylation, capping, fatty acid modifications and including unnatural backbone and/or side chain structures. It will be readily apparent to those of skill in the art that a peptide mimetic comprises the structural continuum between an amino acid chain and a non-peptide small molecule. Peptide mimetics generally retain a recognizable polypeptide-like polymer unit structure. Thus, a peptide mimetic typically retains the function of binding to any target molecule that a natural polypeptide binds to. Other peptidomimetics and methods of making same will be known to those of skill in the art.

The polypeptides can be comprised of D- or L-amino acid residues. Once synthesized, the polypeptides can be modified, for example, by N-terminal acetyl- and C-terminal amide-groups. It is also contemplated that all polypeptides presently described can be made using modified amino acid residues. In certain embodiments, the peptides of the invention may further comprise modifications analogous to post-translational modifications. Such modifications include, but are not limited to, acetylation, carboxylation, glycosylation, phosphorylation, lipidation, and acylation. As a result, the modified peptidomimetics may contain non-amino acid elements, such as polyethylene glycols, lipids, poly- or mono-saccharide, and phosphates. Effects of such non-amino acid elements on the functionality of a polypeptide can be tested using the assay methods disclosed herein

Synthesized polypeptides can be further isolated by HPLC to a purity of at least about 80%, preferably 90%, and more preferably 95%.

The polypeptides described herein can also be expressed recombinantly, especially when the polypeptide does not comprise a “D” amino acid residues. This embodiment relies on routine techniques in the field of recombinant genetics. Generally, the nomenclature and the laboratory procedures in recombinant DNA technology described herein are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. Basic texts disclosing the general methods of use in this invention include Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 1994)).

Polymerase chain reaction or other in vitro amplification methods may also be useful, for example, to clone nucleic acid sequences that code for the polypeptides to be expressed, to make nucleic acids to use as probes for detecting the presence of encoding mRNA in physiological samples, for nucleic acid sequencing, or for other purposes. Nucleic acids amplified by the PCR reaction can be purified from agarose gels and cloned into an appropriate vector.

In another embodiment, a method for develop a new molecular imaging probe, wherein first a new probe is designed or synthesized, an in vitro assay to determine affinity to CD44/Rhamm complexes is carried out, then if affinity for these complexes is of sufficient specificity, the probe is labeled and then tested in vivo. Using such techniques, the leading compound thus far is hyaluronan and HA mimics.

Imaging Component. The imaging component of the probe generally comprises a label. Methods of labeling are well known to those of skill in the art. Preferred labels are those that are suitable for use in in vivo imaging. The anti-Rhamm probes may be detectably labeled prior to detection. Alternatively, a detectable label which binds to the hybridization product may be used. Such detectable labels include any material having a detectable physical or chemical property and have been well-developed in the field of immunoassays.

A label for use in the present invention is any composition detectable by spectroscopic, photochemical, biochemical, immunochemical, or chemical means. Useful labels in the present invention include radioactive labels (e.g., ³²P, ¹²⁵I, ¹⁴C, ³H, and ³⁵S), fluorescent dyes (e.g. fluorescein, rhodamine, Texas Red, etc.), electron-dense reagents (e.g. gold), enzymes (as commonly used in an ELISA), colorimetric labels (e.g. colloidal gold), magnetic labels (e.g. DYNABEADS™), and the like. Examples of labels which are not directly detected but are detected through the use of directly detectable label include biotin and dioxigenin as well as haptens and proteins for which labeled antisera or monoclonal antibodies are available.

The particular label used is not critical to the present invention, so long as it does not interfere with the detection of CD44/Rhamm complexes. However, in a preferred embodiment, the targeting component is a radionuclide (e.g. ¹⁸F, ¹¹C, ¹³N, ⁶⁴Cu, ⁶⁸Ga, ¹²³I, ¹¹¹In, ^{99m}Tc, etc.) due to the ease of using such techniques as SPECT, CT and PET imaging for in vivo detection of CD44/Rhamm complexes and tumor progenitor cells. Decision as to appropriate imaging component for agents used in SPECT or PET imaging can also be determined by whether the radionuclide is generated by generator or cyclotron or is an chelator or organic/halide. The labeled probes may use labels such as positron-emitting tracers, including but not limited to, PET radiopharmaceuticals such as, [¹¹C]choline, [¹⁸F]fluorodeoxyglucose (FDG), [¹¹C]methionine, [¹¹C]choline, [¹¹C]acetate, or [¹⁸F]fluorocholine, which may be applied to imaging in vivo using whole-body PET cameras.

A direct labeled probe, as used herein, is a probe to which a detectable label is attached. Because the direct label is already attached to the probe, no subsequent steps are required to associate the probe with the detectable label. In contrast, an indirect labeled probe is one which bears a moiety to which a detectable label is subsequently bound, typically after the probe is hybridized with the target CD44/Rhamm complexes.

In other embodiments, the presence of CD44/Rhamm complexes is detected by contacting the sample with a first antibody that specifically binds to CD44 and a second antibody that binds to Rhamm. Optionally, the first, second, or both antibodies can be labeled with a detectable label as described above.

In another embodiment, the imaging component is a metal, a semiconductor material, multi-layers of metals, a metal oxide, an alloy, a polymer, or carbon nanomaterials. In certain embodiments the imaging component is a particle comprising a metal selected from the group consisting of Ga, Au, Ag, Cu, AI, Ta, Ti, Ru, Ir, Pt, Pd, Os, Mn, Hf, Zr, V, Nb, La, Y, Gd, Sr, Ba, Cs, Cr, Co, Ni, Zn, Ga, In, Cd, Rh, Re, W, Mo, and oxides, and/or alloys, and/or mixtures, and/or nitrides, and/or sintered matrix thereof.

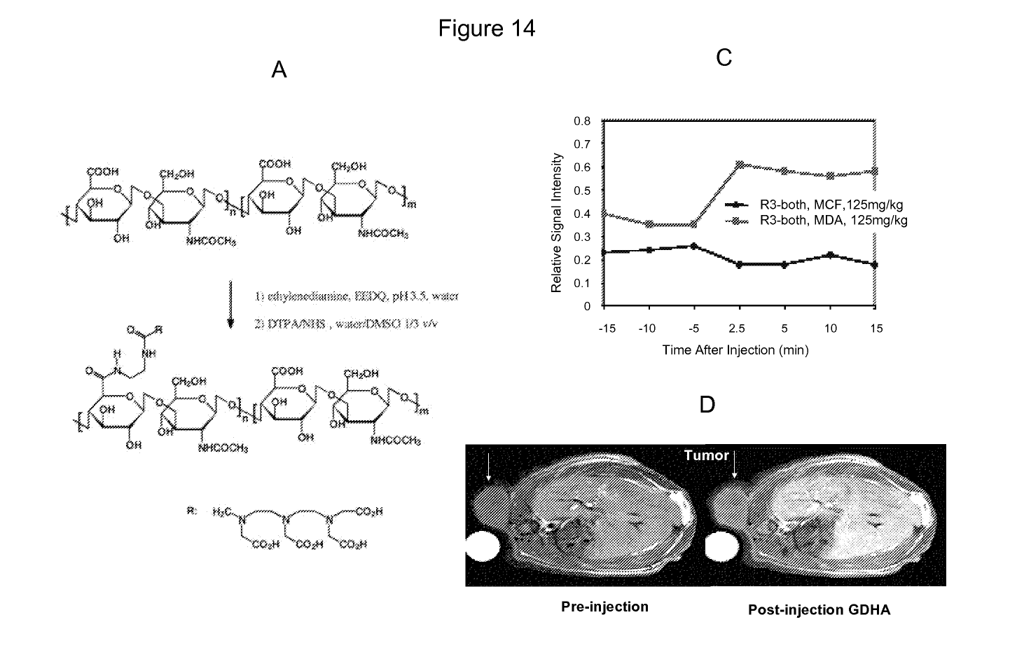

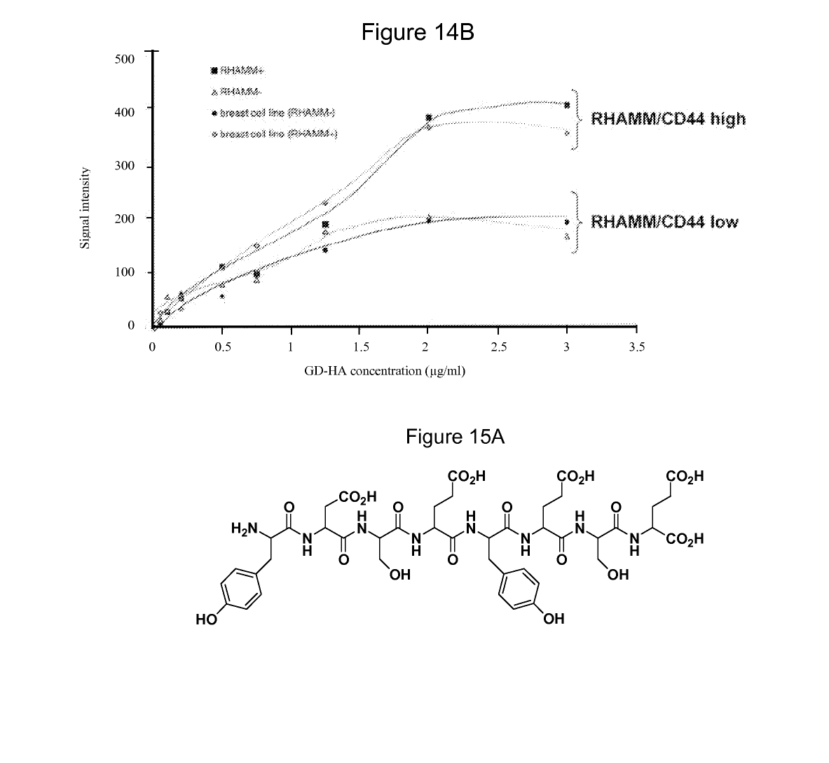

In one embodiment, t may be preferred to use labeled HA as the labeled probe because it is known in the art that HA can be labeled with metals including gadolinium, gold, superparamagnetic Fe₂O₃ beads and CdSe/ZnS quantum dots. See the methods described in Gouin, S, and F. M. Winnik, 2001. Quantitative assays of the amount of diethylenetriaminepentaacetic acid conjugated to water-soluble polymers using isothermal titration calorimetry and colorimetry. Bioconjug Chem. 12(3): p. 372-7, and Shen, F., C. Poncet-Legrand, S. Somers, A. Slade, C. Yip, A. M. Duft, F. M. Winnik, and P. L. Chang, 2003. Properties of a novel magnetized alginate for magnetic resonance imaging. Biotechnol Bioeng. 83(3): p. 282-92, hereby incorporated by reference. FIG. 14A shows a schematic for modification of HA with a metal chelator, DTPA, for the attachment of gadolinium. In another embodiment, the chelator is a chelated paramagnetic ion or a labeled chelator such as DTPA, or DOTA (DOTA=1,4,7,10-tetrakis(carboxymethyl)-1,4,7,10-tetraazacyclododecane), or other known chelators as is known in the art, which can be labeled with metal ions such as Gd³⁺ and ⁶⁴Cu, and other contrast agents for MRI.

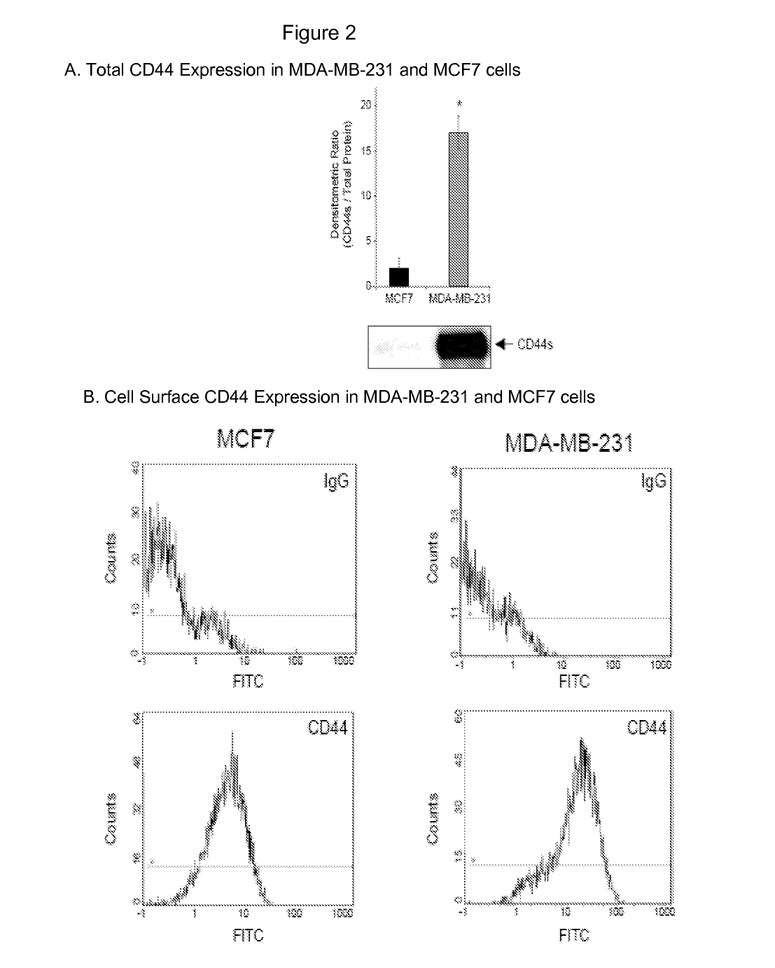

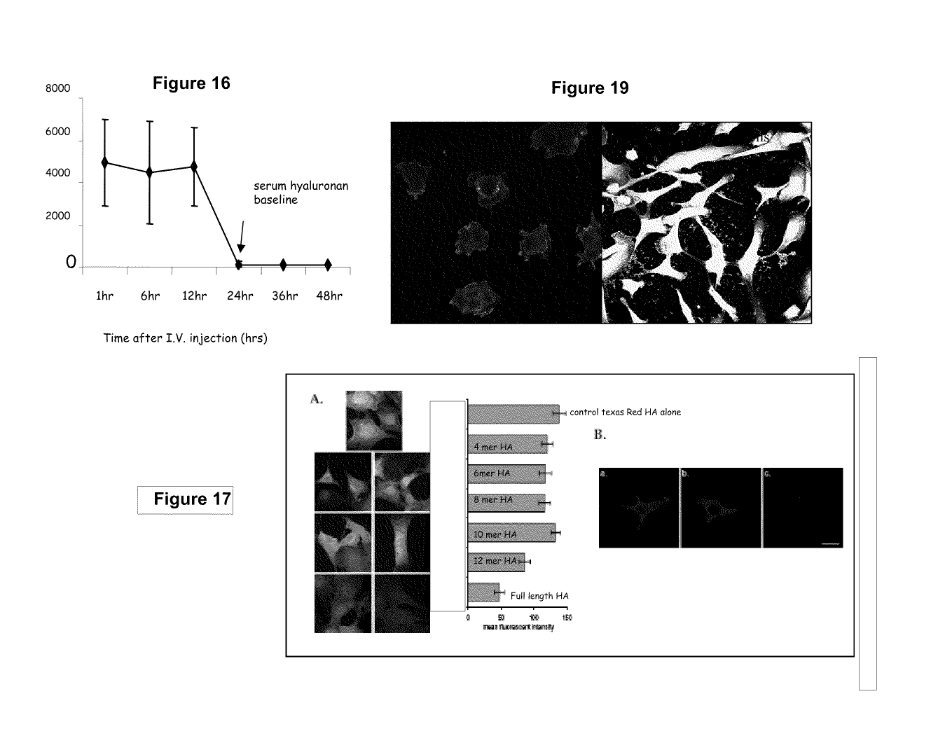

In another embodiment, the presence of CD44/Rhamm complexes is detected by contacting the sample with HA-metal nanoparticles to image highly tumorigenic cells in vivo to diagnose and prognose cancer in patients because it was found that HA alone or HA-gadolinium (GD-HA) nanoparticles administered as intravenous (I.V.) infusions bind preferentially to sites of high HA receptor expression, such as those found in blood vessels, MDA-MB-231 breast tumor xenografts which express high levels of Rhamm and CD44 (FIG. 2) and in the liver whose endothelial cells express the HA endocytic receptor, Hare (FIG. 14D). It was also found that 3-12 mg/kg HA, injected as I.V. infusions into either humans or mice, exhibits a half-life of up to 12 hrs (FIG. 16). Thus, preferential binding in vivo of HA and HA-metal nanoparticles to CD44/Rhamm complexes permits the detection of tumor progenitor cells and enables methods for the diagnosis and prognosis cancer in patients.

In one embodiment, the probe is an HA-metal nanoparticle comprising HA or an HA mimetic, decorated with a metal and a nanoparticle. The nanoparticles (e.g., semiconductor nanocrystals and the like) typically comprise a core and a shell. The core and the shell may comprise the same material or different materials. The shell may further comprise a hydrophilic coating or another group that facilitates conjugation of a CD44/Rhamm complex targeting component to the nanoparticle (i.e., via a linking agent). In some embodiments, the semiconductor nanocrystals comprise a core upon which a hydrophilic coating has been deposited.

The core and the shell may comprise, e.g., an inorganic semiconductive material, a mixture or solid solution of inorganic semiconductive materials, or an organic semiconductive material. Suitable materials for the core and/or shell include, but are not limited to semiconductor materials, carbon, metals, and metal oxides. In a preferred embodiment, the nanoparticles comprise a semiconductor nanocrystal. In a particularly preferred embodiment, the semiconductor nanocrystals comprise a CdSe core and a ZnS shell which further comprises a SiO₂ hydrophilic coating.

Suitable semiconductor materials for the core and/or shell include, but are not limited to, elements of Groups II-VI (ZnS, ZnSe, ZnTe, CdS, CdSe, CdTe, HgS, HgSe, HgTe, MgS, MgSe, MgTe, CaS, CaSe, CaTe, SrS, SrSe, SrTe, BaS, BaSe, BaTe, and the like) and III-V (GaN, GaP, GaAs, GaSb, InN, InP, InAs, InSb, and the like) and IV (Ge, Si, and the like), and alloys or mixtures thereof. Suitable metals and metal oxides for the core and/or shell include, but are not limited to, Au, Ag, Co, Ni, Fe₂O₃, TiO₂, and the like. Suitable carbon nanoparticles include, but are not limited to, carbon nanospheres, carbon nano-onions, and fullerene.

Semiconductor nanocrystals can be made using any method known in the art, hereby listed and incorporated by reference. For example, methods for synthesizing semiconductor nanocrystals comprising Group III-V semiconductors or Group II-VI semiconductors are set forth in, e.g., U.S. Pat. Nos. 5,751,018; 5,505,928; and 5,262,357. The size of the semiconductor nanocrystals can be controlled during formation using crystal growth terminators U.S. Pat. Nos. 5,751,018; 5,505,928; and 5,262,357. Methods for making semiconductor nanocrystals are also set forth in Gerion et al., J. Phys. Chem. 105(37):8861-8871 (2001) and Peng et al., J. Amer. Chem. Soc., 119(30):7019-7029 (1997).

### VI. Inhibiting CD44/Rhamm Inhibits Cancer Cell Metastasis

The invention further provides methods to inhibit cancer cell metastasis by inhibiting CD44/Rhamm. In one embodiment, the method comprises contacting a cancer cell with a compound that inhibits formation of CD44/Rhamm complexes. In one embodiment, the compound would prevent CD44 and Rhamm from forming complexes with one another and/or with HA.

Another embodiment provides for a method for identifying a compound that inhibits cancer cell metastasis. The method comprises contacting a cancer cell with a compound suspected of inhibiting metastasis of cancer cells, and detecting the presence or amount of CD44/Rhamm complexes, whereby a reduction in the amount of CD44/Rhamm complexes identifies the compound as an inhibitor of cancer cell metastasis.

In one embodiment, the cancer cell is in a mammal and further, that mammal is a human. In another embodiment, the cancer cell is biopsied from a subject. The cancer cell is contemplated to be any kind of cancer cell such as a breast cancer cell, a colon cancer cell, a melanoma cancer cell, etc. In a preferred embodiment, the cell is a breast cancer cell.

Compounds that can be used to inhibit formation of CD44/Rhamm complexes include, but are not limited to, an antibody, a small molecule, a mimetic, a peptide, a siRNA, an antisense oligo, or an aptamer. In a preferred embodiment, the CD44/Rhamm inhibitor compound is an antibody. Antibodies that specifically bind or inhibit Rhamm or CD44, may be used to inhibit cancer cell metastasis. Such use of antibodies to treat cancer has been demonstrated by others and may be useful in the present invention to inhibit or down-regulate Ras-MAP kinase pathway activation, which in turn will inhibit CD44/Rhamm complex formation and thereby inhibit cancer cell metastasis.

Another embodiment of the invention is a method for identifying a compound that inhibits cancer cell metastasis. The method comprises contacting a cancer cell with a compound suspected of inhibiting metastasis of cancer cells, and detecting the presence or amount of CD44/Rhamm complexes, whereby a reduction in the amount of CD44/Rhamm complexes identifies the compound as an inhibitor of cancer cell metastasis.

In some embodiments, the compound suspected of being an inhibitor is an antibody, a small molecule, a peptide, a mimetic, a siRNA, an antisense oligo, or an aptamer.

Further methods for identifying such compounds are described in co-pending International patent application, “Modulation of Rhamm (CD168) for Selective Adipose Tissue Development,” which was filed on the same day as this application, incorporated by reference in its entirety.

### VIII. Imaging Progenitor Cell Populations In Vivo

The CD44/Rhamm complex probe of the invention can be administered directly to a mammalian subject using any route known in the art, including e.g., by injection (e.g., intravenous, intraperitoneal, subcutaneous, intramuscular, or intradermal), inhalation, transdermal application, rectal administration, or oral administration. In one embodiment, the CD44/Rhamm complex detecting probe is administered subcutaneously. In another embodiment, the CD44/Rhamm complex detecting probe is administered intravenously. In a preferred embodiment, an effective amount of the CD44/Rhamm complex probe is administered via non-systemic, local administration, such as by peripheral administration which includes peripheral intramuscular, intraglandular, and subcutaneous administration routes, and allowing the probe several hours in vivo to be carried to sites of CD44/Rhamm complexes and tumorigenic cell populations.

The dose administered to a patient, in the context of the present invention should be sufficient to effectively image sites of CD44/Rhamm complexes and tumorigenic cell populations with sufficient specificity for a surgeon to perform a biopsy or other procedure to remove tumorigenic cell populations detected by imaging CD44/Rhamm complexes. The dose will be determined by the efficacy of the particular vector (e.g. peptide or nucleic acid) employed and the condition of the patient, as well as the body weight or surface area of the patient to be treated. The size of the dose also will be determined by the existence, nature, and extent of any adverse side-effects that accompany the administration of a probe in a particular patient.

For administration, CD44/Rhamm complex detecting probes of the present invention can be administered at a rate determined by the LD-50 of the polypeptide or nucleic acid, and the side-effects of the polypeptide or nucleic acid at various concentrations, as applied to the mass and overall health of the patient. Administration can be accomplished via single or divided doses, e.g., doses administered on a regular basis (e.g., daily) for a period of time (e.g., 2, 3, 4, 5, 6, days or 1-3 weeks or more).

In still further embodiments, about 5 to about 2000 LD 50 units of the labeled probe are administered to said subject using recognized clinical standards and practices.

The pharmaceutical compositions of the invention may also comprise a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 17th ed., 1989).

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

Once the probe is administered to the patient, the patient is positioned in an imaging station such as for SPECT, CT, PET, MRI, or NIR imaging. In some embodiments, known and commercially available PET or PET/CT scanners can be used to carry out imaging of tumorigenic cell populations in vivo. Such scanners include those commercially sold by Siemens (e.g., Biograph, ECAT ACCEL, or ECAT EXACT HR+), GE Healthcare (e.g., Discovery PET/CT), and Philips (e.g., CPET, Gemini, or Allegro).

### IX. Assessing Progenitor Cell Populations

In one embodiment, to confirm that the present findings that identification of CD44/Rhamm complexes indicates highly tumorigenic cell populations the following can be carried out as described in Example 6. Primary tumors from advanced cancer patients will be cut into small pieces some of which will be immediately engrafted into mammary fat pads to assess tumorigenicity and to provide additional material for analysis. The remainder of the tumor tissue will be digested with collagenase to obtain single cell suspensions. Cells will be characterized and sorted for a tumorigenic surface phenotype, for example, CD44+/ESA+/CD24−/lineage−, with FACS using the following antibodies obtained from Biomeda (CA): Anti-CD44, Anti-CD24, and anti-ESA. The lineage negative properties of these cells will be verified by the use of the following lineage antibodies by way of example: Anti-CD2, CD3, CD10, CD16, CD18, CD31, CD64, and CD140b. These cell subsets will then be assessed for tumorigenicity by measuring tumor size following transplantation into mammary fat pads of NOD-SCID mice.

This method could be adapted for clinical management of breast or other tumor patients by providing further (e.g. additional to the phenotype of HA uptake, CD44+, Rhamm+ denoting aggressive tumor subsets) evidence of tumor aggression. This method could also be used to identify which treatments are most effective in shrinking the tumors before treating the patient. Imaging progenitor cell populations in vivo also allows a clinician to non-invasively monitor tumor shrinkage, cell progression, metastasis and efficacy of administered therapies.

In another embodiment, it is contemplated that a third therapeutic component is linked, attached or conjugated to the presently described probes. For example, a known cancer therapeutic (e.g., an anti-ErbB2 antibody or an anti-Her2 antibody), alone or coupled with a carrier compound, can be attached to the HA mimetic as a therapeutic component. Delivery to the site of tumorigenic cell populations can be confirmed by imaging in vivo. In one embodiment, the labeled probe having a therapeutic is labeled differently than another probe having only the targeting and imaging component for contrast.

### X. EXAMPLES

### Example 1

### Materials and Methods

Reagents (Antibodies, Growth factors, Hyaluronan and Inhibitors)—Medical grade HA prepared from bacterial fermentation (provided by Hyal Pharmaceutical Co., Mississauga, ON) was free of detectable proteins, DNA or endotoxins. The MW range was approximately 250-300 kD. Primary antibodies used were ERK1 and non-immune IgG (Santa Cruz); phospho-ERK1,2 (Cell Signaling); p21Ras (Oncogene Science, Cambridge, Mass.); CD44 (IM7, Pharmingen); CD44 (Hermes-3, kind gift of Dr. Sirpa Jalkanen, University of Kuopio, Finland). Polyclonal Rhamm antibodies (Zymed, San Diego, Calif.) used in this study were prepared against the following sequences: Antibody-1 was prepared against peptide KSKFSENGNQKN (aa 150-162; SEQ ID NO:11), antibody-2 was against peptide VSIEKEKIDEKS (aa 217-229; SEQ ID NO:12), and antibody-3 against peptide QLRQQDEDFR (aa 543-553; SEQ ID NO:13) of human Rhamm (48,49). Specificity of Rhamm antibodies were determined using Rhamm−/− lysates and peptide competition. Secondary antibodies used were the following: For western blot detection, horseradish peroxidase (HRP)-conjugated anti-mouse (Bio-Rad Laboratories, Hercules, Calif.), anti-rabbit (Amersham, Oakville, ON), and anti-rat (Santa Cruz); For immunofluorescense amalysis, anti-rabbit Alexa 555 and anti-rat Alexa 433 (Molecular Probes). The MEK1 inhibitor, PD098059 (2-[2′-amino-3′-methoxyphenyl]-oxanaphthalen-4-one]) compound, was purchased from Calbiochem Biosciences (Mississauga, ON). An HA binding peptide (YKQKIKHVVKLK; SEQ ID NO:15) was synthesized based upon the sequence reported by Savani et al to block HA-mediated migration of macrophages (Savani, R. C., Hou, G., Liu, P., Wang, C., Simons, E., Grimm, P. C., Stern, R., Greenberg, A. H., DeLisser, H. M., and Khalil, N. (2000) Am J Respir Cell Mol Biol 23, 475-48 and its reported ability to bind to HA (Yang, B., Yang, B. L., Savani, R. C., and Turley, E. A. (1994) Embo J 13, 286-296). A scrambled peptide, YLKQKKVKKHIV (SEQ ID NO:14) was used as a control for the HA-binding peptide.

Cell Culture—Human breast carcinoma cell lines MDA-MB-231 and MCF7 were obtained from American Type Culture Collection (Manassas, Va.) and were cultured in Dulbecco's Modified Eagle's Medium (DMEM) (Gibco BRL, Burlington, Ontario) supplemented with 10% (v/v) heat-inactivated fetal bovine serum (FBS) (Hyclone Laboratories Inc., Logan, Utah) and 10 mM HEPES (Sigma Chemical Co., St. Louis, Mo.), at pH 7.2 Immortalized normal human breast epithelial cell lines MCF10A transfected with the empty pH106 plasmid containing the neomycin resistance gene and MCF10A cells transfected with the human mutant H-Ras oncogene (mutated at G12-V12) were a kind gift of Dr. Channing Der (North Carolina) and grown as previously described (46,47). Briefly, the cells were grown in DMEM/F-12 (1:1) supplemented with 5% equine serum, 0.1 μg/mL cholera toxin, 10 μg/mL insulin (Gibco BRL), 0.5 μg/mL hydrocortisone (Sigma) and 0.02 μg/mL epidermal growth factor (Collaborative Research Inc., Palo Alto, Calif.). All cultures were incubated in a humidified atmosphere of 5% CO₂ at 37° C.

Western Immunoblotting—Cells plated at 50% subconfluency for 12 hours were washed with ice-cold phosphate buffered saline (PBS) and lysed in ice-cold RIPA buffer (25 mM Tris-HCl, pH 7.2, 0.1% SDS, 1% Triton-X-100, 1% sodium deoxycholate, 0.15M NaCl, 1 mM EDTA, and 50 nM HEPES [pH 7.3]) containing the protease inhibitors leupeptin (1 μg/mL), phenylmethylsulfonyl fluoride (PMSF, 2 mM), pepstatin A (1 g/mL), aprotinin (0.2 TIU/mL) and 3,4-dichloroisocoumarin (200 mM), sodium orthovanadate, and 1 mM NaF (Sigma Chemical Col, St. Louis, Mo.). Cell lysates were then micro-centrifuged at 13,000×g for 20 minutes at 4° C. (Heraeus Biofuge 13, Baxter Diagnostics, Mississauga, ON) after standing for 20 minutes on ice. Protein concentrations of the supernatants were determined using the DC protein assay (Bio-Rad). 10 μg of total protein from each cell lysate was loaded and separated by electrophoresis on a 10% SDS-PAGE gel together with prestained molecular weight standards (Gibco BRL). Following electrophoresis, proteins were transferred to nitrocellulose membranes (Bio-Rad) in a buffer containing 25 mM Tris-HCl (pH 8.3), 192 mM glycine and 20% methanol using electrophoretic transfer cells (Bio-Rad) at 100V for 1.5 hour at 4° C. Additional protein binding sites on the membrane were blocked with 5% defatted milk in TBST (10 mM Tris base (pH 7.4), 150 mM NaCl, and 0.1% Tween-20 (Sigma)). The membranes were incubated with the primary antibody for Rhamm, CD44, Ras or ERK1,2 (all diluted at 1:1000 or 1 μg/mL in 1% defatted milk in TBST) for 2 hours at room temperature. The membranes were washed three times at 15 minute intervals with 1% defatted milk in TBST. Immunodetection was performed using secondary antibodies conjugated to HRP (diluted 1:5000 or 1 mg/mL) in 1% defatted milk in TBST for 1 hour at room temperature followed by three washed with TBST. Blotting was visualized by the enhanced chemiluminescence (ECL) Western blotting detection system (Amersham Pharmacia Biotech, Piscataway, N.J.) according to the manufacturer's instructions. Quantification of optical densities of the reactive protein bands was performed on a Bio-Rad Video Densitometer. The specificity of the anti-Rhamm antibody was confirmed by probing blots with either non-immune rabbit IgG, or anti-Rhamm antibody pre-incubated with Rhamm fusion protein as stated above. To account for variations in loading, parallel SDS gels were carried out with the experiments and equal amounts of the protein were separated on these gels. These other gels were then stained with Coommassie blue dye in order to confirm equal loading. The densitometric results were presented as a mean of three experiments±standard deviations.

Immunoblot analysis of EGF stimulated ERK1,2 activation −5×10⁴ MCF7 and MDA-MB-231 cells were plated in complete growth medium (DMEM, 10% FCS) on 6 cm cell culture plates and allowed to attach for 4 hours. The growth medium was replaced by defined medium (DMEM, 4 μg/ml insulin, 8 μg/ml transferrin). After overnight culture, cells were stimulated with 20 ng/ml EGF (Sigma) in defined medium. For antibody blocking experiments, cell surface Rhamm and/or CD44 function was blocked by pre-incubating cells for 30 minutes in the presence of either anti-CD44 antibody (IM-7, 10 μg/ml), anti-Rhamm antibody (10 μg/ml), IgG (10 μg/ml) or a combination of anti-CD44 and anti-Rhamm antibodies prior to EGF stimulation. EGF stimulation, protein isolation and SDS-PAGE were performed as described above. For immunoblot analysis, antibodies were used at following dilutions: anti-phospho-ERK1,2 (Sigma) at 1:3000, anti-ERK1,2 (Santa Cruz) at 1:3000, anti-rabbit secondary (Bio Rad) at 1:4000, and anti-mouse secondary (Bio Rad) at 1:4000. Quantification was performed as described above and the ratio pERK1,2:total ERK1,2 was calculated. Statistical analysis is based on triplicate samples with significance level P<0.05.

Measurement of HA production—Cells were plated at sub-confluence in DMEM+ 10% FCS for 12 hours, which was replaced with serum free medium for 24-48 hours. HA released into the medium was collected and assayed using an ELISA assay (Amersham Pharmacia Biotech, Piscataway, N.J.) as per the manufacturer's instructions.

Fluorescence Activated Cell Sorting (FACS)—Cells were grown to 50% subconfluence on 15 cm culture plates in growth media, 12 hours after subculturing, and rinsed in Ca²⁺-free Hank's Buffered Saline Solution (HBSS)/20 mM HEPES, pH 7.3. Cells were harvested with non-enzymatic HBSS-based cell dissociation solution (Sigma) and resuspended in 5 mL cold PBS and centrifuged at 1200 rpm for 3 minutes. Cells were washed in another 5 mL cold PBS and then blocked in cold 10% FCS/HBSS/HEPES (FACS buffer) for 30 minutes. The viability of released cells was established to be between 85% and 95%, by Trypan blue exclusion. For detection of cell surface Rhamm, an aliquot of 2×10⁶ cells was incubated with anti-Rhamm antibody (1:100, 1 μg/μL) in a total volume of 200 μl of FACS buffer for 30 minutes on ice, and washed three times in cold FACS buffer. Rabbit IgG (1:100 of 1 μL/mL) was used as a negative control for each cell line. Fluorescein isothiocyanate (FITC)-conjugated goat anti-rabbit IgG (1:300 dilution, Sigma) in FACS buffer was then added and incubated for 30 minutes in the dark on ice. The cells were washed again and examined with a flow cytometer (Beckman Coulter) using FACS Calibur with Cell Quest acquisition and analysis software (Becton Dickinson, Lincoln Park, N.J.). For detection of cell surface CD44, 1×10⁶ cells were incubated with 1 μg anti-CD44 antibody (clone IM7, Pharmingen) or 1 μg rat IgG antibody (Santa Cruz) in PBS/2% BSA for 1 hour on ice after which time they were washed with cold PBS/2% BSA. Cells were then incubated with rabbit anti-rat Alexa 488 (diluted 1:100, Molecular Probes) in PBS/2% BSA for 1 hour on ice. Cells were washed in cold PBS/2% BSA. Cells were resuspended in 1 mL fresh, cold PBS/2% paraformaldehyde (Sigma) and were stored overnight at 4° C. Before flow cytometric analysis, samples were filtered (cell strainer caps, Becton Dickinson Labware). Data was collected using a Beckman Coulter flow cytometer. Viable cells were gated based on forward and side scatter to eliminate dead aggregates and debris, and then the distribution of fluorescence intensity was calculated.

Immunoprecipitation assays and in vitro pulldown binding assays—Co-immunoprecipitation analyses were performed using 400 μg of protein from each cell lysate mixed with 5 μg of either anti-Rhamm, anti-CD44, anti-ERK1, anti-rabbit IgG (for polyclonal antibodies), or anti-mouse IgG antibodies (for monoclonal antibodies). After 12 hours of incubation at 4° C. on a rotator, 25 μl of a 50% suspension of protein A/G-Sepharose beads (Gibco BRL) was added to each tube and the samples were mixed end-over-end for another 4 hours at 4° C. The beads were pelleted by brief centrifugation at 7000×g and washed three times with cold 0.5% Triton-X-100/PBS. Bound proteins were released from the beads by heating the samples in 25 μl of 2× Laemmli buffer for 5 minutes. Protein samples were subjected to 12% SDS-PAGE and immunoblotted as described above.

In vitro pulldown binding assays were performed using recombinant Rhamm protein (63 kDa isoform) that was purified as a GST fusion protein as previously described (53). Briefly, 1 mg of cellular lysate was incubated with recombinant Rhamm-GST or recombinant GST protein on glutathione sepharose beads (Amersham) overnight at 4° C. Beads were then pelleted by centrifugation and washed five times with 1 mL of cold lysis buffer. Bound proteins were released from the beads by heating the samples in 25 μl of 2× Laemmli buffer for 5 minutes. Protein samples were subjected to 10% SDS-PAGE and immunoblotted as described above.

Time-Lapse Cinemicrography—Random motility analyses were performed to quantify the effect of the blocking Rhamm and CD44 antibodies, HA binding peptide, hyaluronan, and the MEK1 inhibitor, PD098059 on cell motility. Cells were seeded on T-25 flasks (Costar, Cambridge, Mass.) at 1×10⁵ cells/flask. Cells were incubated with anti-Rhamm antibody (30 μg/ml), anti-CD44 antibody (30 μg/ml), a mixture of anti-Rhamm (30 μg/ml) and anti-CD44 antibodies (30 μg/ml), HA binding peptide (1 μg/ml) or its scrambled control (1 μg/ml), and/or 50 μM PD098059 for 30 minutes prior to filming. Alternatively, cells were stimulated with 50 μg/L of HA immediately prior to filming. As a control, a mixture of mouse and rabbit IgG (30 μg/ml each), DMSO (for PD098059) or PBS (for HA) were used. Cell locomotion was monitored for a period of 6 hours using a 10× modulation objective (Zeiss, Germany) attached to a Zeiss Axiovert 100 inverted microscope equipped with Hoffman Modulation contrast optical filters (Greenvale, N.Y.) and a 37° C. heated stage. Cell images were captured with a CCD video camera module attached to a Hamamatsu CCD camera controller. Motility was assessed using Northern Exposure 2.9 image analysis software (Empix Imaging, Mississauga, Ontario). Nuclear displacement of 20-30 cells was measured and data were subjected to statistical analysis (see below). Each experiment was repeated at least three times. PD098059 MEK inhibitor was used to test the involvement of the MAP kinase pathway in the motility of these cells. The cells were incubated with the MEK1 inhibitor at 50 μM in complete culture medium 30 minutes before the beginning of motility filming. DMSO alone was used as a control. The results of motility analyses were expressed as means (μm/hour) means±standard deviations, unless otherwise indicated.

Confocal Microscopy—MCF7 and MDA-MB-231 cells were plated sparsely (approximately 5000 cells/well) on coverslips in a 24-well dish. The cells were incubated overnight in D-MEM supplemented with 10% fetal calf serum (FCS) in a humidified atmosphere of 5% CO₂ at 37° C. Cells were rinsed briefly with 1% BSA/TBS and were then fixed in fresh 3.7% paraformaldehyde in TBS for 10 minutes at room temperature. Cells were rinsed with 1% BSA/TBS and were then permeabilized with 0.5% Triton X-100 in 1% BSA/TBS for 15 minutes at room temperature. Cells were again rinsed in 1% BSA/TBS and blocked in 5% FCS in 1% BSA/TBS for 1 hour at room temperature. For the phospho-ERK1,2/CD44 double staining, cells were incubated with anti-phospho p44/p42 MAP kinase (Thr202/Tyr204, Cell Signaling Technology) and anti-CD44 (IM7, BD Pharmingen) antibodies, each diluted 1/100 in 1% BSA/TBS, for 1 hour at room temperature. For the Rhamm/CD44 double staining, cells were first incubated with an anti-Rhamm antibody, diluted 1/100 in 1% BSA/TBS, overnight at 4° C. After the overnight incubation, cells were further incubated with the anti-CD44 antibody (IM7, BD Pharmingen), diluted 1/100 in 1% BSA/TBS, for 1 hour at room temperature. After incubation with primary antibodies, cells were rinsed four times for 10 minutes each in 1% BSA/TBS. Primary antibodies were then visualized by incubating cells with anti-rabbit Alexa 555 (Molecular Probes) and anti-rat Alexa 488 (Molecular Probes), diluted 1/150 in 1% BSA/TBS, for 1 hour at room temperature. After incubation with secondary antibodies, cells were rinsed four times for 10 minutes each in 1% BSA/TBS. Cells were briefly incubated with DAPI (4′,6-Diamidino-2-phenylindole) diluted in 1% BSA/TBS and then mounted (IF mounting medium, DAKO) on slides. A Zeiss LSM510 Meta Multiphoton confocal microscope was used to visualize the cells (Dept. Anatomy and Cell Biology, UWO).

Statistical analysis—Statistically significant (p<0.05) differences between means were assessed by the unpaired, two-tailed Student's t-test method. Cell motility was based on means of >20 cells per experiment and western blot quantification was based on triplicate samples, unless otherwise indicated. Hyaluronan production was reported as a mean of 10 separate cultures.

### Example 2

### Highly Invasive Breast Tumor Cell Lines Produce High Levels of Endogenous HA that Sustains their Rapid Motility

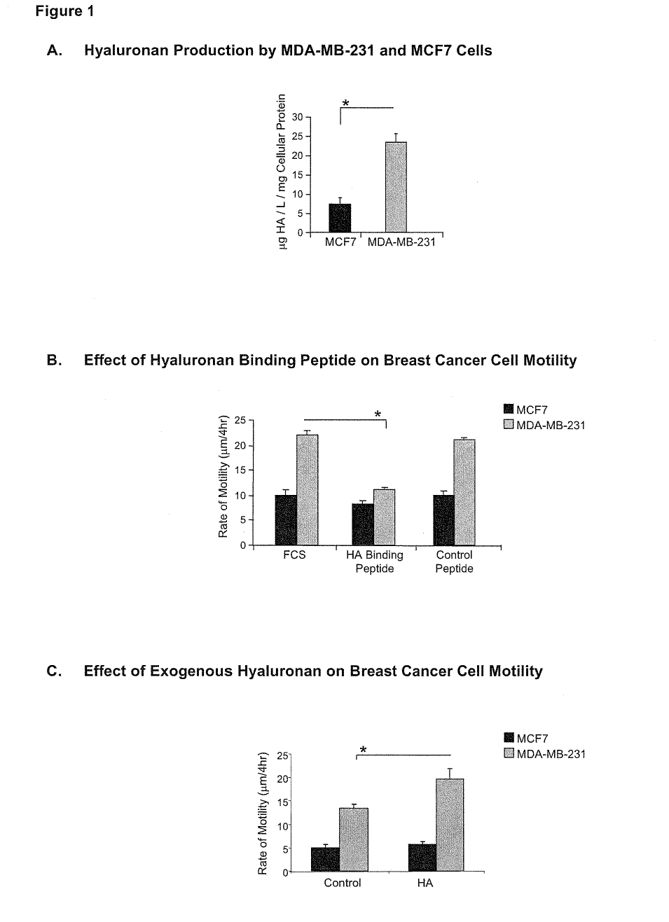

HA, a motogenic factor that is strongly correlated with clinical progression of breast cancer, has been linked in numerous studies to both ERK1,2 activation and tumor cell motility/invasion. Therefore, we first examined the potential importance of endogenous HA in sustaining the motility of breast tumor cell lines in vitro. HA levels in the medium of cultured cells were significantly higher in MDA-MB-231 cells than in MCF7 cells (FIG. 1A); similar differences were also observed between cultures of Ras-MCF10A and MCF10A cells (data not shown). A synthetic peptide mimicking Rhamm sequence (YKQKIKHVVKLK; SEQ ID NO:15) that was previously shown to bind to HA and inhibit HA-mediated macrophage motility (51) was tested for its effects on motility of the breast cancer cell lines. Exposure of MDA-MB-231 cells to this HA-binding peptide significantly reduced motility (FIG. 1B) but had no effect on MCF7 cells (FIG. 1B). A scrambled control peptide (YLKQKKVKKHIV; SEQ ID NO:14), which does not affect HA-mediated macrophage motility (51), had no effect on the motility of either cell line. To compare the responsiveness of the cell lines to exogenous HA, cultures were serum-starved for 24-48 hours to reduce endogenous levels of HA production (<1 ng/ml) (FIG. 1C). The addition of 25-50 μg/ml exogenous HA significantly stimulated motility of serum starved MDA-MB-231 cells (FIG. 1C) but had no effect on the motility rate of MCF7 cells. These results indicate that invasive breast tumor cell lines such as MDA-MB-231 cells have established an HA dependent autocrine mechanism in the presence of growth factors (e.g. serum supplemented medium) that sustain high levels of motility. By contrast, poorly invasive breast cancer cells (e.g. MCF7) lack both the ability to produce high levels of HA and the ability to respond to exogenous HA provided in their microenvironment.

### Example 3

### MDA-MB-231 and Ras-MCF10A Breast Tumor Cells Express Cell Surface CD44 and Rhamm and Higher Levels of Cell Surface Rhamm than Either MCF7 or MCF10A Cells

Since the invasive breast cancer cell lines respond in an autocrine fashion to HA, we next evaluated the potential importance of CD44 and Rhamm in mediating this response as both receptors have been implicated in the motility and invasion of breast cancer cells, and in motogenic response to HA. CD44 and Rhamm protein expression was quantified by both flow cytometry and western blot analysis. Levels of total CD44s protein (FIG. 2A) and cell surface CD44 (FIG. 2B) were significantly higher in MDA-MB-231 than MCF7 cells. Similar differences in CD44 were also observed between Ras-MCF10A cells and their parental, non-transformed counterparts (Suppl. FIG. Ia and data not shown).

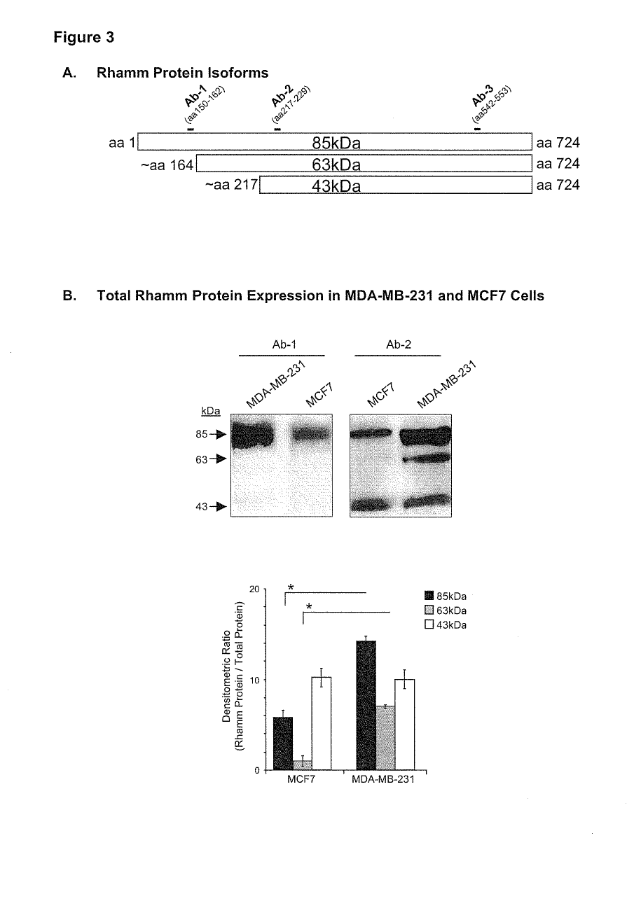

Western blot analysis using an anti-Rhamm antibody that recognizes all Rhamm protein forms (FIG. 3A, Ab-3, data not shown) revealed the presence of multiple immuno-reactive bands in both MDA-MB-231 and MCF7 cells. Rhamm protein expression consisted primarily of two major protein species of 85 kD and 43 kD (FIG. 3B, Ab-2; Ab-3, data not shown), while a 63 kD protein form was expressed at low levels. In contrast, MDA-MB-231 cells expressed high levels of all three protein species (FIG. 3B, Ab-2; Ab-3, data not shown). The densitometric ratios of each of the Rhamm protein isoforms/total Rhamm protein (obtained by totaling the densitometric values of each Rhamm immunoreactive band recognized by Ab-2) were significantly higher in MDA-MB-231 than in MCF7 cells (FIG. 3B). The 63 kD Rhamm protein that is highly expressed by MDA-MB-231 cells corresponds to an oncogenic Rhamm isoform (Hall, C. L., et al., Overexpression of the hyaluronan receptor RHAMM is transforming and is also required for H-ras transformation. Cell, 1995. 82(1): p. 19-26) that is expressed in many invasive human tumor cells (Turley, E. A., P. W. Noble, and L. Y. Bourguignon, Signaling properties of hyaluronan receptors. J Biol Chem, 2002. 277(7): p. 4589-92) and that is transforming in fibroblasts. Furthermore, this molecular weight is similar to a cell surface form of Rhamm expressed by macrophages (Zaman, A., et al., Expression and role of the hyaluronan receptor RHAMM in inflammation after bleomycin injury. Am J Respir Cell Mol Biol, 2005. 33(5): p. 447-54). In order to further determine what the Rhamm protein forms expressed in these cells were or to determine where each of the isoforms started, Rhamm antibodies that were specific for different regions of the Rhamm protein were used. Rhamm antibody-1 (FIG. 3A, Ab-1), which is specific for a sequence within the N-terminal region of Rhamm, only reacted with the 85 kD full-length protein (FIG. 3B). However, Rhamm antibody-2 (FIG. 3A, Ab-2), recognized 85 kD, 63 kD, and 43 kD isoforms in both cell lines, suggesting that the 63 and 43 kD isoforms are N-terminal truncated Rhamm proteins. Furthermore, this provides additional evidence that the highly expressed 63 kD isoform seen in many human tumours and in the aggressive cell lines is the same Rhamm protein form that was transforming when overexpressed in fibroblasts.

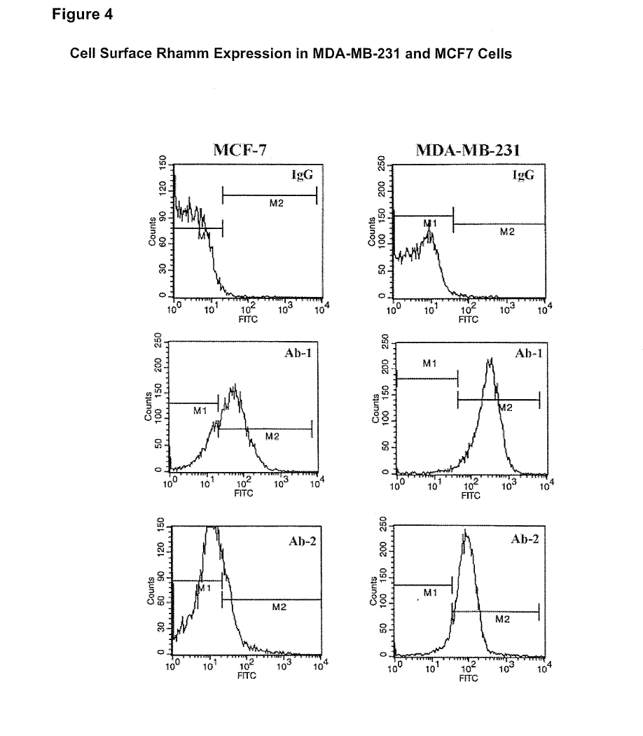

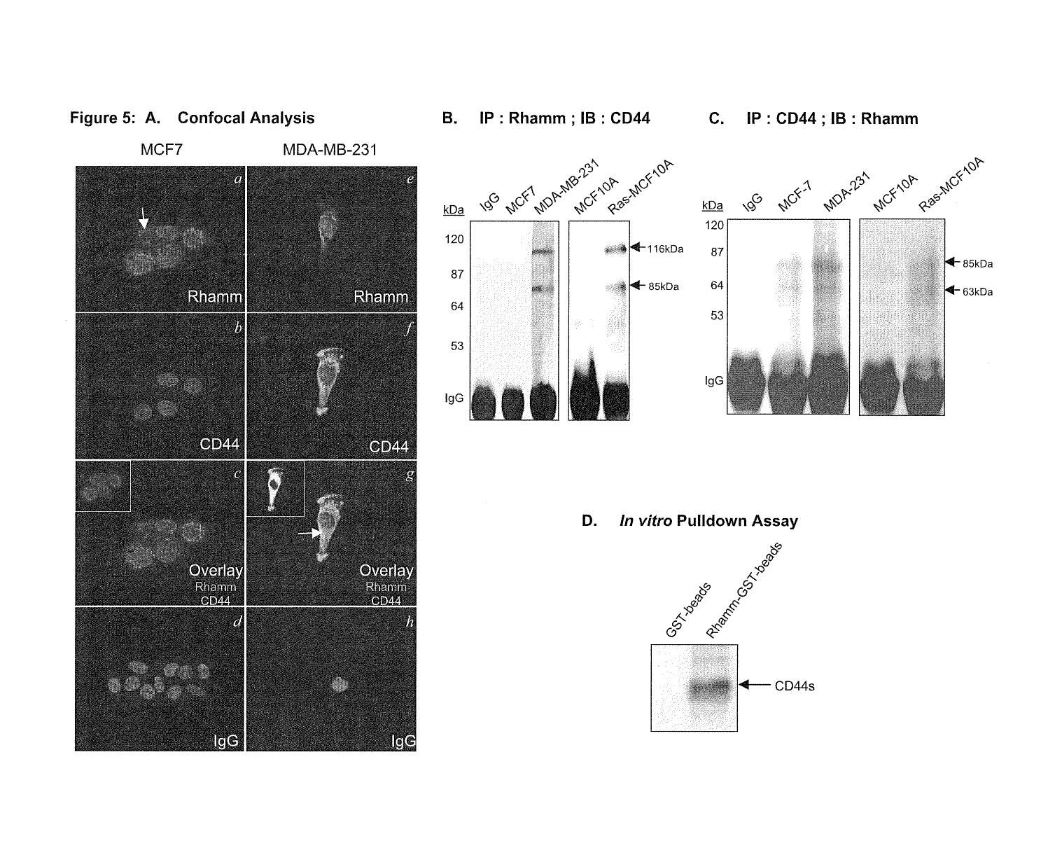

MDA-MB-231 cells expressed high levels of cell surface Rhamm as detected by flow cytometry, while MCF7 cells lacked detectable cell surface Rhamm (FIG. 4). Furthermore, the 85, 63, and 43 kD isoforms were all detected on the surface of the MDA-MB-231 cells (FIG. 4). Confocal analysis of Rhamm expression in these two cell lines confirmed different subcellular compartmentalization of Rhamm protein suggested by FACS analysis (FIG. 5A). Rhamm appeared largely intracellular in MCF7 and decorated the cytoskeleton, likely interphase microtubules (FIG. 5A, panel a). In contrast, Rhamm appeared in cell processes and the perinuclear area of MDA-MB-231 cells (FIG. 5A, panel e). Similar quantitative and qualitative differences in Rhamm expression were also observed when comparing Ras-MCF10A and parental MCF10A cells (Suppl. FIG. 1b). In particular, only Ras-MCF10A cells expressed cell surface Rhamm as detected by flow cytometry (data not shown).

Confocal analysis of dual CD44 and Rhamm staining confirmed the low CD44 protein expression in MCF-7 cells and revealed a co-distribution of these two HA receptors in cell processes and in the peri-nuclear region of MDA-MB-231 cells (FIG. 5A, panel g; panel inset shows enhancement of co-localization in white). The co-association of Rhamm and CD44 was further investigated using immunoprecipitation assays (FIG. 5B, C) and in vitro pull-down binding assays (FIG. 5D). Anti-Rhamm antibodies co-immunoprecipitated two major CD44 isoforms in MDA-MB-231 and Ras-MCF10A cells, including a species with a similar mobility to the standard 85 kD protein (CD44s) and an additional band of approximately 120 kD, which likely represents a splice variant or post-translationally modified form (FIG. 5B). Anti-Rhamm antibodies did not co-immunoprecipitate detectable CD44 proteins in either MCF7 or MCF10A cells (FIG. 5B and data not shown). In the recipricol assay, anti-CD44 antibodies co-immunoprecipitated detectable levels of both 63 and 85 kD Rhamm isoforms in the invasive MDA-MB-231 and Ras-MCF10A cells and to a much lesser extent in the non-invasive MCF7. Anti-CD44 antibodies only co-immunoprecipitated the full-length 85 kD Rhamm protein in the non-turmoigenic, parental MCF10A cells (FIG. 5C). An ability of the 63 kD Rhamm protein, which likely represents a cell surface form of Rhamm (Soule, H. D., et al., Isolation and characterization of a spontaneously immortalized human breast epithelial cell line, MCF-10. Cancer Res, 1990. 50(18): p. 6075-86), to associate with CD44 was confirmed using recombinant Rhamm-GST protein (63 kD isoform)-Sepharose in pull-down assays. The 63 kD recombinant Rhamm protein co-associated with CD44s and a higher molecular weight protein form from MDA-MB-231 lysates only (FIG. 5D). These results suggest that high expression of both CD44 and Rhamm at the cell surface, where they also likely co-associate, are associated with HA responsiveness and an aggressive tumorigenic phenotype. Since CD44 has previously been shown to promote motility of breast cancer cell lines and Rhamm has been shown to promote motility of fibroblasts, we next compared the relative roles played by these HA receptors in the motility of the fibroblastic MDA-MB-231 and Ras-MCF10A tumor cells with the epithelial MCF7 and MCF10A cells, using function blocking antibodies specific to each of these proteins.

### Example 4

### CD44 and cell surface Rhamm are Necessary for Motility of MDA-Mb-231 and Ras-MCF10A Cells but not for MCF7 or MCF10A Cells

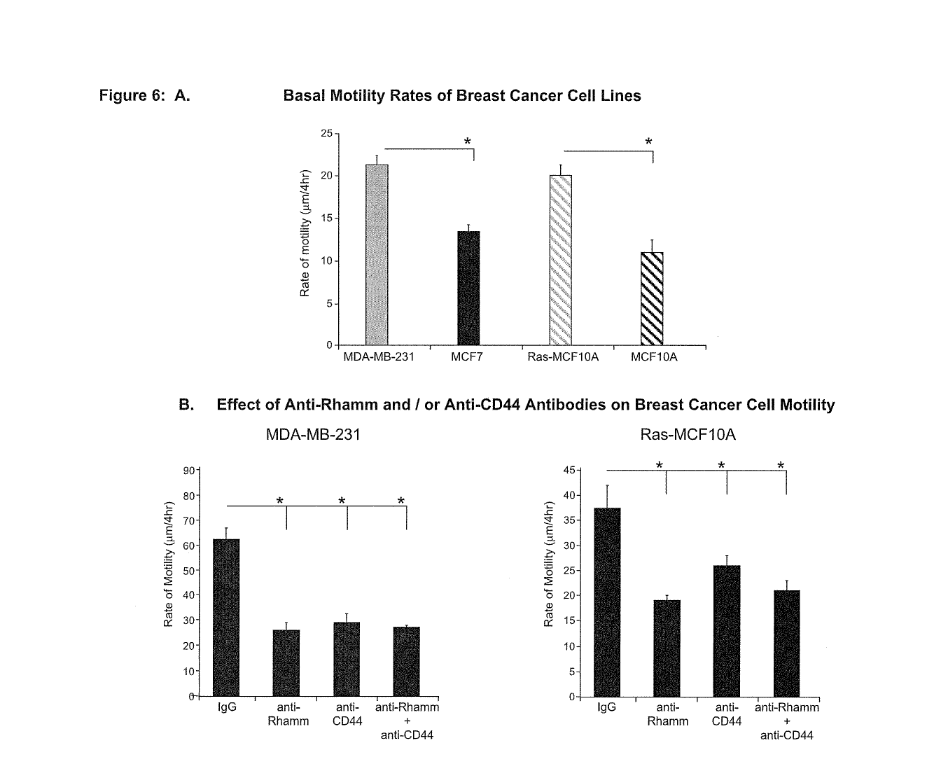

In order to be invasive, tumor cells must acquire the ability to migrate (1,2). We therefore first compared the relative motility rates of invasive and non-invasive cell lines and analyzed whether differences in motility are related to differences in HA receptor expression. MDA-MB-231 and Ras-MCF10A cells were significantly more motile than either MCF7 or MCF10A cells (FIG. 6A). We used inhibitory antibodies against CD44 and Rhamm, alone or in combination, to assess their relative roles in the high rate of motility of MDA-MB-231 and Ras-MCF10A cells. Motility of both of these cell lines was significantly inhibited by both anti-CD44 and anti-Rhamm antibodies (FIG. 6B). However, the addition of both antibodies together had no additive inhibitory effect (FIG. 6B). Similar results were observed after adding anti-CD44 and/or anti-Rhamm antibodies to Ras-MCF10A cells (FIG. 6B). In contrast, these inhibitory antibodies had only minor effects on the motility of either MCF7 or MCF10A cells (data not shown). These results indicate that both CD44 and cell surface Rhamm contribute to high motility rates of aggressive breast cancer cell lines, appearing to act on the same motogenic pathway, but are much less important for the motility of poorly invasive breast tumor cell lines.

Sustained activation of ERK1,2 motogenic pathways are important factors in promoting invasive and metastatic behavior of the above aggressive breast cancer cell lines (39-41). Since both CD44 and cell surface Rhamm regulate ERK1,2 activity (49,58), their role in sustaining the activation state of ERK1,2 and ERK1,2-regulated motility in aggressive breast cancer cell lines was next assessed.

### Example 5

### CD44 and Rhamm Occur as Complexes with ERK1,2 and Both HA Receptors are Required for Sustained ERK1,2 Activation and for ERK1,2-Mediated Motility in Invasive Breast Cancer Cell Lines

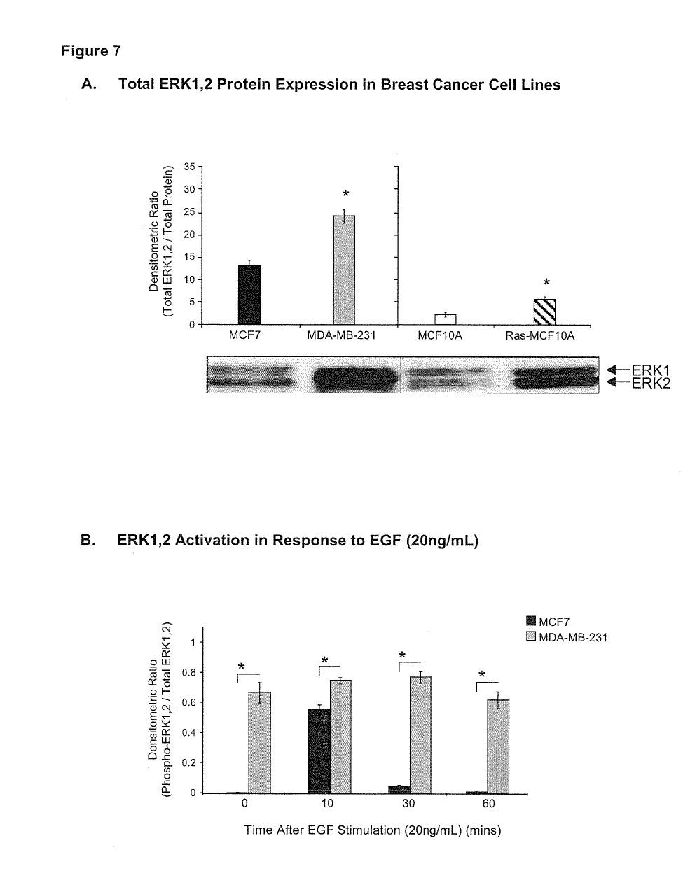

Both MDA-MB-231 and Ras-MCF10A cells were confirmed to express higher levels of total ERK1,2 protein than either MCF7 or MCF10A cells (FIG. 7A) (47,59). Under standard culture conditions, MDA-MB-231 cells also exhibited significantly higher levels of constitutively active (phospho-) ERK1,2 than MCF7 cells (FIG. 7B), as did Ras-MCF10A cells vs. parental MCF10A cells (data not shown). The high activity levels of ERK1,2 in MDA-MB-231 and Ras-MCF10 were associated with expression of mutant active Ras (H-Ras) (data not shown).

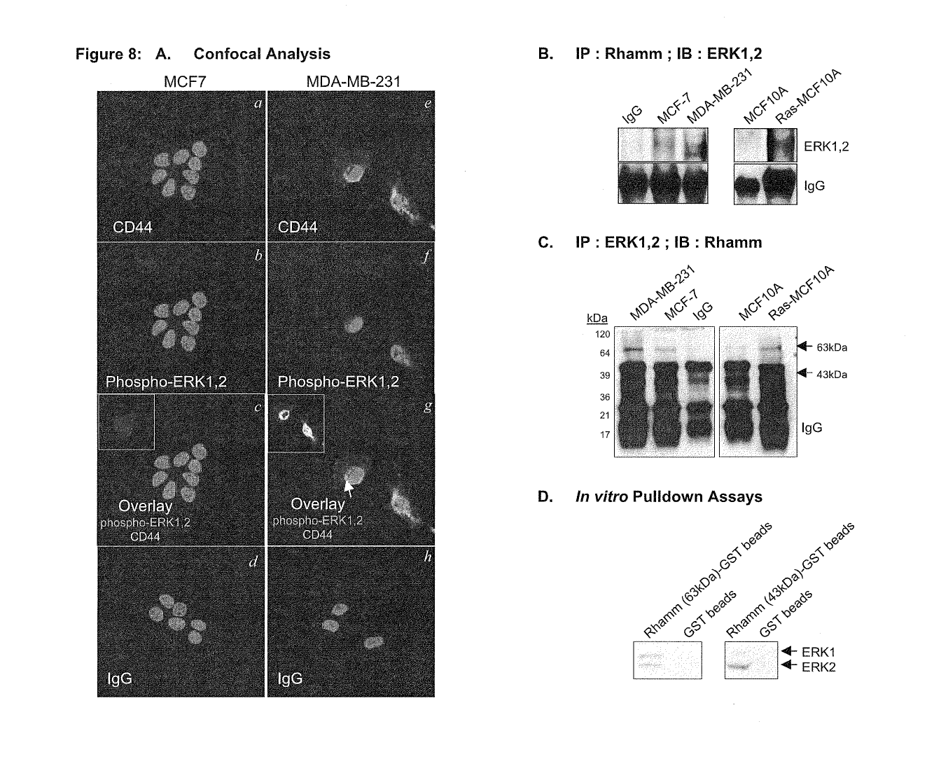

MDA-MB-231 and MCF7 tumor cells differed in their ability to activate ERK1,2 in response to EGF, a growth factor linked to breast cancer progression. MDA-MB-231 cells, which have been reported to express high endogenous levels of EGF (Martinez-Carpio, P. A., et al., Constitutive and regulated secretion of epidermal growth factor and transforming growth factor-beta1 in MDA-MB-231 breast cancer cell line in 11-day cultures. Cell Signal, 1999. 11(10): p. 753-7), maintained significantly higher ERK1,2 activity than MCF7 cells in serum free medium (FIG. 7B). The addition of EGF did not further increase ERK1,2 activity in MDA-MB-231 cells. In contrast, the addition of EGF to MCF7 cells increased ERK1,2 activation, which reached maximal levels by 10 minutes then dropped to baseline by 30 minutes (FIG. 7B). However, the activation of ERK1,2 in MCF7 cells was significantly less than that of MDA-MB-231 cells even when maximally stimulated by EGF. Thus, activation of ERK1,2 was transient in MCF7 while MDA-MB-231 cells sustained high levels of ERK1,2 activity throughout the experimental period. These differences correlated with co-localization of CD44/Rhamm with ERK1,2 in MDA-MB-231 and MCF7 cells (FIG. 8A). Active ERK1,2 and CD44 co-localized as vesicular structures in the perinucleus and to a more limited extent in the nucleus of MDA-MB-231 cells (FIG. 8A, panel g; panel inset shoes enhanced co-localization as white). These results imply that the majority of the CD44/Rhamm/activated ERK1,2 complexes occur as vesicles near the nucleus. ERK1,2 were co-immunoprecipitated by anti-Rhamm antibodies in all cell lines (FIG. 8B). However, although ERK1,2 co-immunoprecipitated with Rhamm in all cell lines, only the 63 kD and not the 85 kD (full length) Rhamm protein form was detected in western blots (FIG. 8C). The ability of ERK1,2 to co-immunoprecipitate with the 43 kDa Rhamm protein form was not clear (FIG. 8C) so in vitro pulldown assays were used to assess this possible association. Recombinant Rhamm-GST protein (both the 63 kDa and 43 kDa isoforms) were able to pull down ERK1 and ERK2 from MDA-MB-231 cell lysates in this assay (FIG. 8D), suggesting that both Rhamm protein forms could associate with ERK1,2 in culture. The role of these HA receptors in sustaining high consititutive ERK1,2 activation in MDA-MB-231 cells was next assessed.

MDA-MB-231 cells maintained in standard culture conditions were exposed to isotype-matched non-immune IgG and either anti-Rhamm or anti-CD44 antibodies, and the effect on ERK1,2 activity was quantified (FIG. 9A). ERK1,2 activity was significantly reduced in MDA-MB-231 cells in the presence of anti-Rhamm antibodies (FIG. 9A). Anti-CD44 antibodies also reduced ERK1,2 activity but the effect did not reach a significance level of p<0.05. The addition of both inhibitory antibodies together had no greater inhibitory effect on ERK1,2 activation compared to either antibody alone (FIG. 9A).

These results show that ERK1,2 associates with Rhamm/CD44 complexes and that both HA receptors regulate activation of these MAP kinases. Since ERK1,2 activity is required for motility and invasion of breast cancer cell lines such as MDA-MB-231 cells,

We next assessed if these HA receptors mediate motility rates via an ERK1,2 dependent pathway in these cells. As expected, the addition of the MEK1 inhibitor PD098059 alone significantly inhibited the basal motility rate of MBA-MB-231 cells (FIG. 9B). Anti-Rhamm antibodies also significantly inhibited the basal motility of these highly invasive cells but it had no greater inhibitory effect on motility when added in the presence of the MEK1 inhibitor (FIG. 9B). In contrast, neither anti-Rhamm nor the MEK1 inhibitor had any detectable effect on the basal motility of MCF7 cells. Similar results were observed using an inhibitory anti-CD44 antibody in the presence or absence of the MEK1 inhibitor (data not shown). These results suggest that Rhamm and CD44-regulated ERK1,2 activity is required for the high constitutive motility of MDA-MB-231 cells but is not required for basal motility of MCF7 cells.

Collectively, these results suggest that although Rhamm can complex with both ERK1,2 and CD44 in all of the breast cancer lines examined, the formation of these complexes and their role in promoting ERK1,2 activation and cell motility in the less aggressive breast cancer cell lines is limited by cell surface display of CD44. These results are therefore consistent with the hypothesis that CD44/Rhamm/ERK1,2 complexes sustain high basal ERK1,2 activity, which in turn drives a high rate of basal motility typical of the aggressive breast cancer cell lines.

### Example 6

### Using HA Uptake as a Mechanism to Identify Tumor-Initiating Progenitor Cells

As shown in the previous Examples, the two HA receptors, CD44 and Rhamm, co-associate and are also functionally linked to promote invasion and anchorage-dependent survival. Importantly, such cell lines internalize exogenous HA more rapidly than less tumorigenic breast tumor cell lines that do not bear progenitor markers and that express low levels of CD44 or Rhamm (e.g. FIG. 14B). HA uptake by MDA-MB-231 cells requires Rhamm and CD44, as uptake is blocked by either anti-CD44 or anti-Rhamm antibodies, which specially inhibit HA binding to each receptor (FIG. 14B).

We have developed methods for decorating HA with metals including gadolinium, gold, superparamagnetic Fe₂O₃ beads and CdSe/ZnS quantum dots. Such methods are described in Gouin, S, and F. M. Winnik, 2001. Quantitative assays of the amount of diethylenetriaminepentaacetic acid conjugated to water-soluble polymers using isothermal titration calorimetry and colorimetry. Bioconjug Chem. 12(3): p. 372-7; and Shen, F., C. Poncet-Legrand, S. Somers, A. Slade, C. Yip, A. M. Duft, F. M. Winnik, and P. L. Chang, 2003. Properties of a novel magnetized alginate for magnetic resonance imaging. Biotechnol Bioeng. 83(3): p. 282-92, both references which are hereby incorporated by reference.

Further, we have shown that 3-12 mg/kg HA, injected as I.V. infusions into either humans or mice, exhibits a half-life of up to 12 hrs, and that HA alone or HA-gadolinium (GD-HA) nanoparticles administered as I.V. infusions bind preferentially to sites of high HA receptor expression, such as those found in blood vessels (Thierry, B., F. M. Winnik, Y. Merhi, J. Silver, and M. Tabrizian, 2003. Bioactive coatings of endovascular stents based on polyelectrolyte multilayers. Biomacromolecules. 4(6): p. 1564-71, and Thierry, B., F. M. Winnik, Y. Merhi, and M. Tabrizian, 2003. Nanocoatings onto arteries via layer-by-layer deposition: toward the in vivo repair of damaged blood vessels. J Am Chem. Soc. 125(25): p. 7494-5, hereby incorporated by reference), MDA-MB-231 breast tumor xenografts which express high levels of Rhamm and CD44 (FIG. 2A) and the liver, whose endothelial cells express the HA endocytic receptor, Hare (See FIG. 14D and Weigel, P. H. and J. H. Yik, 2002. Glycans as endocytosis signals: the cases of the asialoglycoprotein and hyaluronan/chondroitin sulfate receptors. Biochim Biophys Acta. 1572(2-3): p. 341-63).

A major goal is to determine the potential efficacy of using HA-metal nanoparticles to image highly tumorigenic cells with this phenotype in vivo with the longer-term goal of developing this technology to better image and diagnose tumours in patients. Subsets of the highly tumorgenic CD44⁺/CD24^{-/low}/lineage⁻/ESA⁺ breast tumor cells should also selectively express high levels of Rhamm protein and that as a consequence of this surface phenotype, these tumor cells subsets will likely internalize HA more rapidly than surrounding normal/less tumorigenic cells. Therefore, the use of HA-metal conjugates should permit detection of these highly tumorigenic subsets.

HA-drug or liposome complexes have been used to target to sites of high HA receptor expression in vivo. See for example, Thierry, B., F. M. Winnik, Y. Merhi, J. Silver, and M. Tabrizian, 2003. Bioactive coatings of endovascular stents based on polyelectrolyte multilayers. Biomacromolecules. 4(6): p. 1564-71; Mastrobattista, E., R. H. Kapel, M. H. Eggenhuisen, P. J. Roholl, D. J. Crommelin, W. E. Hennink, and G. Storm, 2001. Lipid-coated polyplexes for targeted gene delivery to ovarian carcinoma cells. Cancer Gene Ther. 8(6): p. 405-13; and Eliaz, R. E., S, Nir, C. Marty, and F. C. Szoka, Jr., 2004. Determination and modeling of kinetics of cancer cell killing by doxorubicin and doxorubicin encapsulated in targeted liposomes. Cancer Res. 64(2): p. 711-8; the methods and compositions of which are hereby incorporated by reference. However, the use of HA nanoparticles for the imaging and detection of tumorigenic progenitor cells has not yet been described.

HA is normally present in serum in minute amounts (15 ng/ml) and the T^{1/2} of such serum HA is in the order of minutes. However, this half-life is significantly prolonged in humans when HA is infused at high (1-12 mg/kg or serum 20-300 μg/ml) concentrations or coated onto surfaces of liposomes. References describing such methods include Peer, D. and R. Margalit, 2004. Loading mitomycin C inside long circulating hyaluronan targeted nano-liposomes increases its antitumor activity in three mice tumor models. Int J. Cancer. 108(5): p. 780-9; Yerushalmi, N. and R. Margalit, 1998. Hyaluronic acid-modified bioadhesive liposomes as local drug depots: effects of cellular and fluid dynamics on liposome retention at target sites. Arch Biochem Biophys. 349(1): p. 21-6; Eliaz, R. E., S, Nir, C. Marty, and F. C. Szoka, Jr., 2004. Determination and modeling of kinetics of cancer cell killing by doxorubicin and doxorubicin encapsulated in targeted liposomes. Cancer Res. 64(2): p. 711-8; and Eliaz, R. E. and F. C. Szoka, Jr., 2001. Liposome-encapsulated doxorubicin targeted to CD44: a strategy to kill CD44-overexpressing tumor cells. Cancer Res. 61(6): p. 2592-601, all of which are hereby incorporated by reference.

Importantly, we have shown that GD-HA nanoparticles infused IV into nude tumor bearing mice is sufficiently retained in the circulation to permit targeting and deposition within tumor xenografts of which can be detected with MRI (FIG. 14C and FIG. 14D).

The present Example consists of three sections: 1) prepare and characterize uptake of HA-metal nanoparticles (GD-HA and HA-QD/ferrous liquids) in a model of a progenitor, highly tumorigenic human breast cancer cell line (MDA-MB-231) in vitro and in vivo; 2) isolate primary tumor subsets exhibiting a tumorigenic surface phenotype (CD44+/ESA+/CD24−/lineage−) and assess these subsets for Rhamm (CD168) and HA uptake; and 3) assess progenitor capabilities of the highly tumorigenic subsets that rapidly take up HA.

1. Preparation and Characterization of HA Nanoparticles in MDA-MD-231

There are two goals associated with this section: A) to prepare HA-metal nanoparticles optimized for selective uptake into breast cancer cell lines in vivo that express the highest level of HA receptors and progenitor markers; B) to develop superparamagnetic reagents (e.g. ferrofluids) that will permit rapid and selective magnetic separation of breast cancer cells subsets exhibiting high HA uptake.

Methods: The GD-HA nanoparticles were prepared via a three-step procedure as described in Gouin, S, and F. M. Winnik, 2001. Quantitative assays of the amount of diethylenetriaminepentaacetic acid conjugated to water-soluble polymers using isothermal titration calorimetry and colorimetry. Bioconjug Chem. 12(3): p. 372-7. Briefly, the procedure involves (1) the attachment of ethylenediamine to the glucuronic carboxylic acid groups; (2) linking diethylenetriaminepentaacetic acid (DTPA) units to the terminal amine groups DTPA-HA (FIG. 14A); and (3) treatment the DTPA-HA with Gd³⁺. The final step has to occur quantitatively, relative to the level of DTPA incorporation, to ensure that all the DTPA chelates linked to HA are converted to Gd³⁺ complexes and that GD-HA is devoid of highly toxic free Gd³⁺ This was achieved by performing a quantitative titration of a solution of GdCl₃ into a solution of HA-DTPA, for which the level of DTPA conjugation had been measured by isothermal titration calorimetry. The Gd³⁺ content of the resulting GD-HA samples ranged from 9.2 to 15.9 weight % for polymers of molecular weight ˜100,000 daltons (Da). Thus in the most decorated sample, approximately up to 33% of the disaccharide units of HA bear a GD DTPA function. Furthermore, when adsorbed onto a solid surface (e.g. bead) such as might occur upon uptake into cells, GD-HA exhibits superior signal intensity to free GD (data not shown). Using GD-HA (9.2%, Mw100,000 Da), we have obtained a signal intensity within MDA-MB-231 cells that is 35-50% higher than that obtained with free GD. More importantly, MDA-MB-231 xenografts exhibit three-fold greater signal intensity than the less aggressive, non-progenitor MCF-7 breast cancer cell line (FIG. 14C).

These preliminary results lead us to advance the hypothesis that breast stem cells which acquire a tumor initiating function and express CD44 also express elevated levels of Rhamm, amongst other tumorigenic progenitor cell markers. The tumorigenicity of human breast cancer cell lines also correlates with an enhanced ability to internalize exogenous HA.

This signal intensity is nevertheless several fold lower than that of liver tissue and we will endeavour to increase both the signal intensity and selectivity of GD-HA uptake by optimizing the percent decoration of HA with GD and using HA of varying molecular weights. GD-HA or complexes will be prepared following procedures already mastered for modifications of high molecular weight (HMW) HA but the methodology will be applied towards the preparation of HA-metal complexes of controlled size in order to achieve more selective targeting of progenitor cells and to facilitate uptake in vivo. The structural characteristics and purity of the GD-HA samples will be assessed by ¹H and ¹³C NMR spectroscopy, colorimetric assays, gel permeation chromatography (GPC) coupled with multiangle laser light scattering (MALLS), UV and RI detection. HA fragments of defined molecular weight will be prepared by hyaluronidase degradation of high MW HA carried out under strictly controlled timing and conditions.

In addition commercial HA samples of selected size (e.g. 40 kDa and 120 kDa) will be employed. HA samples and polymers of, for example, 5, 10, 40, 120, and 600 kDa will initially be tagged with Texas red. The purpose of these experiments is to optimize the molecular weight of the HA oligomers used for subsequent studies, since our initial studies suggest that oligomers in the range of 30 kD are most effectively internalized by MD-MB-231 cells. The uptake of Texas red-HA oligomers will be monitored by flow cytometry, which can yield a highly quantitative estimate of the extent of HA internalization. Furthermore, labelled breast tumor cells will be evaluated by confocal microscopy to verify that labelled HA is intracellular. Hyaluronidase treatment of labelled cells may also be used in conjunction with both flow cytometry and confocal microscopy to determine the relative percentage of label associated with the extracellular plasma membrane and/or any potential pericellular HA matrix that might be elaborated by the tumor.

To address whether a lower molecular weight HA might better penetrate the tumor for improved imaging, we first determined the smallest size of HA that is taken up in a Rhamm/CD44 dependent fashion (FIG. 17). These studies have indicated that a 12 mer of HA, which has a weight of 2100 daltons, is the smallest size of HA able to detectably compete with full length HA for uptake into Rhamm and CD44 expressing cells. We then prepared GD-HA that has been depolymerized by hyaluronidase to an average molecule weight of 10,000 daltons and also purchased purified HA fragments of 9,580 daltons (40 mer; LifeCore Biosciences, MN) which will decorated with GD to varying extents (9-50%).

As an alternative approach, we have also synthesized 7 mer HA mimetic peptides, described and shown in Example 2 above, to address tumor penetration, since these peptide mimic HA in their ability to bind to Rhamm and possibly CD44, and should easily penetrate tumor xenografts in vivo. These peptides can compete with 350 kDa HA for binding to Rhamm and so may be better taken up by tumor progentiro cells that are embedded in a microenvironment containing HA than HA fragments themselves.

The different genetic backgrounds of MDA-MB-MDA-231 and MCF-7 may make data interpretation difficult. Therefore, we will screen our HA-metal reagents using parental MDA-MB-231 cells as an example of a highly tumorigenic progenitor like-breast cancer cell line, and E-cadherin transfected MDA-MB-231 cells, to provide a less tumorigenic counterpart to the parental cells and “reverted” or differentiated MDA-MB-231 cells achieved in vitro using blocking antibodies to β1 integrin (or inhibitors of MAP Kinase and PI3Kinase) combined with transfection of E-cadherin to select for those reagents that are most rapidly and selectively taken up into the parental MDA-MB-231 cells. Reagents will initially be selected using the above cell line combinations grown in 3D MATRIGEL and allowed to invade or differentiate into ductal epithelial cells as we have described. Aggregates will be harvested from the gels at 4° C. and then initially incubated with various concentrations of GD-HA, then washed and fixed in glutaraldehyde. The cell aggregates will be sent for measurement of GD-HA uptake using MRI.

It is contemplated that preparation of smaller labelled HA fragments will increase tumour labelling due to better penetration of the tumor mass. It is also contemplated to increase the density for GD decoration of HA up to one Gd3+ molecule per hexasaccharide which is likely the optimal degree of decoration as we have shown for Texas Red HA that still interacts with HA receptors. The GD-HA nanoparticles with the highest and most selective tumor cell uptake profiles in vitro will be used to image tumour xenografts in NOD-SCID mice. For these experiments, we will implant varying numbers of parental vs. E-cadherin transfected MDA-MB-231 cells or MDA-MB-231 cells forced to differentiate into ductal epithelial cells and cultured as aggregates in Matrigel plugs, into mammary fat pads of NOD-SCID mice.

Animals will be infused I.V. with selected GD-HA preparations into the tail or penile vein and uptake into the liver and tumor xenografts will be measured at timed intervals after injection an example of which is shown in FIG. 14D. Although the liver gives the strongest signal intensity for uptake of circulating GD-HA, this does not preclude detecting the weaker tumour signal. However, if this becomes an issue for other imaging procedures, uptake of HA into liver endothelial cells can be blocked by pre-infusion of animals with chrondroitin sulfate.

Concurrently, HA-iron oxide complexes will be prepared, starting from high molecular weight HA (˜600 kDa) as well as HA oligomers (˜5 kDa) using methodology established for the preparation of alginate based ferrofluids and described in Shen, F., C. Poncet-Legrand, S. Somers, A. Slade, C. Yip, A. M. Duft, F. M. Winnik, and P. L. Chang, 2003. Properties of a novel magnetized alginate for magnetic resonance imaging. Biotechnol Bioeng. 83(3): p. 282-92. Initial results indicate that this route can be easily applied to HA, as anticipated from the fact that HA and alginate share similar structural features. Next, we will proceed towards the synthesis of HA-quantum dot (QD) complexes, with the goal of taking advantage of the superior photophysical properties, in particular the higher quantum yield and light stability of QDs, compared to organic dyes. QD of various sizes (hence emission properties) will be prepared as described in Lovric, J., et al., Differences in subcellular distribution and toxicity of green and red emitting CdTe quantum dots. J Mol Med, 2005. 83(5): p. 377-85) and complexed with thiol (SH) functionalized HA as described in Shu, X. Z., Y. Liu, F. Palumbo, and G. D. Prestwich, 2003. Disulfide-crosslinked hyaluronan-gelatin hydrogel films: a covalent mimic of the extracellular matrix for in vitro cell growth. Biomaterials. 24(21): p. 3825-34).

Concurrently, we will prepare HA-superparamagnetic QD complexes, using methods such as that described in Wang, D., J. He, N. Rosenzweig, and Z. Rosenzweig, 2004. Superparamagnetic Fe2O3/CdSe ZnS quantum dot core shell particles for cell separation. Nanoletters (January) and hereby incorporated by reference. These luminescent/magnetic HA-tagged nanoparticles can serve not only to visualize cells that overexpress HA, but also to isolate them by passage through a magnetic cell separator. They will be used both to image tumors in vivo and to isolate tumor cell subsets using magnetic separation procedures. It is anticipated that uptake of these complexes will facilitate the purification of defined tumor cell subsets, however, it remains to be seen if the HA complexes that are optimally internalized will be of sufficient mass to effectively image heterogeneous subsets of tumor cells in more complex samples.

2. Identification of Cell Subsets from Primary Tumors

The objective of this specific aim is to identify cell subsets from primary breast tumors that are highly tumorigenic (CD44+/ESA+/CD24−/lineage−), assess whether or not these exhibit enhanced HA uptake and express Rhamm.

Methods: Fresh primary breast tumor samples (from 10-20 advanced breast cancer patients available/yr) will be obtained from the London Regional Cancer Centre, London Ontario Canada in collaboration with Dr. F. Pererra, a Radiation Oncologist, who will collect the samples and ensure that they are rapidly delivered to the Turley laboratory on ice. Primary tumors from advanced breast cancer patients will be cut into small pieces some of which will be immediately engrafted into mammary fat pads to assess tumorigenicity and to provide additional material for analysis. The remainder of the tumor tissue will be digested with collagenase to obtain single cell suspensions. Cells will be characterized and sorted for a tumorigenic surface phenotype (CD44+/ESA+/CD24−/lineage−) with FACS using the following antibodies obtained from Biomeda (CA): Anti-CD44, Anti-CD24, and anti-ESA. The lineage negative properties of these cells will be verified by the use of the following lineage antibodies purchased from PharMingen: Anti-CD2, CD3, CD10, CD16, CD18, CD31, CD64, and CD140b. These cell subsets will then be assessed for tumorigenicity by measuring tumor size following transplantation into mammary fat pads of NOD-SCID mice.

Cell subsets confirmed to be highly tumorigenic will be incubated for several hours at 37° C. with Texas red or QD/ferrous liquid-HA, washed and sorted by FACS to enrich for those with highest uptake of the fluorochrome. Primary tumor cells exhibiting high Texas Red HA uptake will then be analyzed and sorted by both FACS for expression of cell surface Rhamm (CD168) using antibodies prepared in the Turley laboratory and shown to be specific for Rhamm by screening against Rhamm−/− cells (available from the Rhamm knockouts made). Sorted cell subsets exhibiting high HA uptake will be further purified using magnetized particles. Isolated cell subsets will be assessed for their tumorigenic potential using the above mammary fat pad injection xenograft model and their tumor initiating capacity will be compared to the population exhibiting a CD44+/ESA+/CD24−/lineage− surface phenotype.

Using the present methods, the presence of highly tumorigenic cell subsets that likely express the following surface phenotype: CD44⁺/CD24^{-/low}/lineage⁻/ESA⁺ subsets (representing 2-5% of tumor mass) can be detected, but if insufficient starting material limits the number of progenitor cells isolated, we will expand progenitor cells either in culture or in mammary fat pad tissue. Mice injected with sorted tumor cells will be monitored weekly for tumor growth. Once we have determined the tumorigenic potential and surface phenotype of primary tumor cell subsets that rapidly take up Texas Red HA, we will transplant these as xenographs in Nod Scid mice as described above and quantify uptake of GD-HA into primary tumors using MRI. We will also assess the role of Rhamm and/or CD44 in the uptake of GD-HA into tumor cell subsets in vitro using either Rhamm function blocking monoclonal antibodies or siRNA to knockdown expression of either of these HA receptors.

3. Assessment of Highly Tumorigenic Cell Subsets as Progenitor Cells.

The objective of this specific aim is to determine whether or not the primary breast tumor cell subsets identified in section 2 are progenitor cells.

Methods: Cell subsets identified in the previous section 2 to be highly tumorigenic and further characterized for their HA uptake and cell surface phenotype will be isolated in larger numbers by utilizing supra-paramagnetic iron-decorated HA prepared as described in previous section 1. If other characteristics are associated with tumor initiating activity, these cells will be sorted further for the surface characteristics most closely linked to tumorigenic potential. Once isolated, these tumorigenic cell subsets will be cultured as aggregates in Matrigel gels containing EGF and inhibitors of (31 integrin and ERK kinase (PD98059) conditions that we have previously shown force tumor cells to either differentiate or die. Tumor cells that are able to differentiate into ductal epithelium, judged by the presence of Keratin 18/19, ESA markers will be further characterized for expression of other stem cell markers in order to define the nature of the progenitor cells responsible for the tumorigenic potential and to permit further refinement of reagents for identifying and ultimately treating these subsets. We will utilize these HA-metal complexes to determine the smallest number of cells that can be detected both in culture and in vivo using the methods described in the preceding two aims, with goals of testing the efficacy of using HA-metal complexes for in vivo imaging in patients.

### Example 7

### SPECT/CT Imaging HA Uptake as a Mechanism to Identify Tumor-Initiating Progenitor Cells In Vivo

Since MRI is not as sensitive as SPECT-CT cameras in detecting small populations of cells, we will also label HA mimetics, which are not detected by the HARE receptors of the liver, or tagged HA fragments which are smaller than those required for uptake by HARE receptors (e.g. 4-timers) but which still bind to Rhamm (but not CD44) labeled with I¹²³, inject HA preparations into the thigh to prevent rapid metabolism in the liver and image tumors several hours after injection of HA imaging agent. Several hours later tumor xenografts are measured at timed intervals after injection, an similar example of which is shown in FIG. 14D which shows GD-HA uptake.

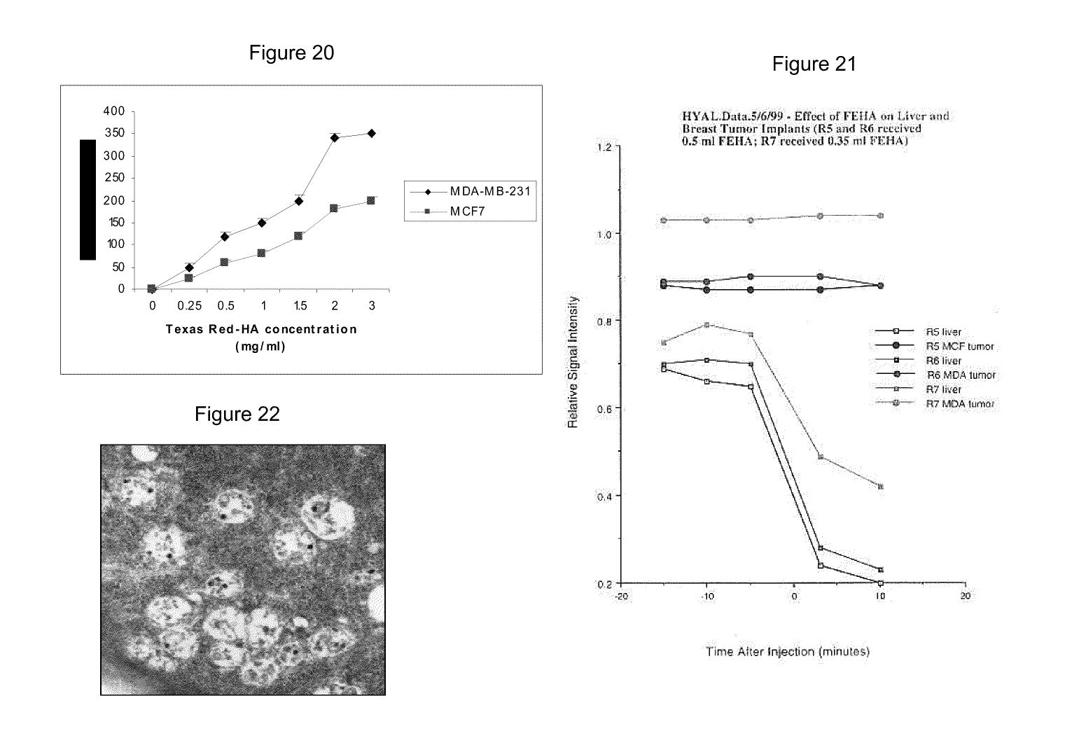

Preparation of Metal Tags: Methods for Preparing Gadolinium-Hyaluronan (GD-HA) nanoparticles and hyaluronan coated colloidal nanoparticles have recently been described in Gouin, S, and F. M. Winnik, Quantitative assays of the amount of diethylenetriaminepentaacetic acid conjugated to water-soluble polymers using isothermal titration calorimetry and colorimetry. Bioconjug Chem, 2001. 12(3): p. 372-7, hereby incorporated by reference. We originally prepared and used GD-HA and Iron (FE) HA nanoparticles to image MDA-MB-231 tumors grown as xenografts on the flanks of immune compromised rats. GD-hyaluronan uptake into MDA-MB-231 and MCF7 breast tumor cells were grown as aggregates in collagen gels, exposed to GD-HA and uptake was quantified with MRI. The MDA-MB-231 cell line was used as an example of a CD44+/CD24− breast tumor progenitor cell while the MCF7 cell line was used as an example of a non-progenitor luminal A breast tumor cell. As shown in FIG. 20, GD-HA uptake was greater in MDA-MB-231 than in MCF tumor cell aggregates consistent with our hypothesis that HA is selectively and rapidly taken up by breast tumor progenitor cells.

MDA-MB-231 tumor xenografts were next grown on the flanks of immune-compromised rats, GD-HA was administered I.V. and 10-20 min later, animals were imaged with MRI. As shown in FIG. 14D, GD-HA was taken up by both the liver and tumor while uptake into other tissues was low. FE-HA was also selectively taken up by liver and tumor tissue (FIG. 21). Uptake is considered to be specific since it is limited to tissues that are known to express high levels of HA receptors: liver endothelium express HARE receptors^{[18-20, 28]} while the tumor expresses both CD44 and RHAMM. However, neither GD-HA nor FE-HA uptake into the tumor is high and in the case of GD-HA the uptake appears to be primarily limited to the tumor edges. Since tumor progenitor cells have been calculated to represent 1-5% of a primary tumor, the results of these experiments suggest that GD-HA/MRI likely do not have high enough tumor penetration or resolution to probe the sub-populations. We therefore next prepared Gold-HA (Au-HA) nanoparticles for use in CT imaging, which provides higher practical resolution than current MRI instruments.

Another advantage of using Au is its ability to enhance ionizing radiation-mediated DNA destruction: Au-HA thus offers the dual potential of acting as both an imaging and therapeutic agent. Au-HA nanoparticles were prepared as described for GD-HA nanoparticles. We monitored binding and uptake into tumor cells using transmission electron microscopy respectively (FIG. 22). These results show that Au-HA is endoctyosed by tumor cells. Experiments designed to quantify uptake and to image tumors in vivo using CT are ongoing. While both of the above modalities (MRI or CT) will eventually provide useful clinical information, they currently lack the detection sensitivity that is required for targeting and analysis of our tumor subpopulations. Thus we next used fluorescence modality that has the highest sensitivity (molecular resolution) and the widest detection span, i.e. from molecular to anatomical.

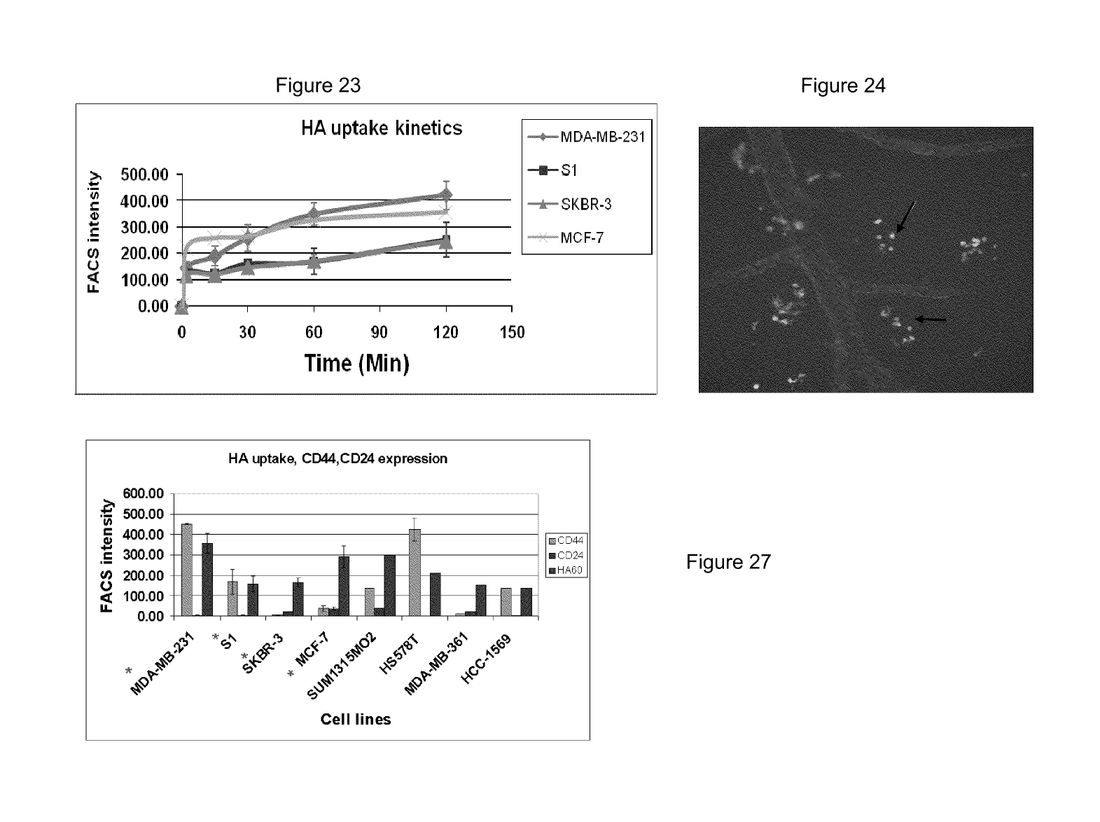

Fluorescent Tags: Texas Red, Alexa Fluor 488, Alexa Fluor 647 and Cy5.5-HA. Fluorochromes were linked to soluble HA (MWe=350,000 daltons) as described in Collis, L., et al., Rapid hyaluronan uptake is associated with enhanced motility: implications for an intracellular mode of action. FEBS Lett, 1998. 440(3): p. 444-9 hereby incorporated by reference, with some modification. Fluorescent-HA uptake by MDA-MB-231 and MCF-7 breast cancer cells was compared using both confocal microscopy and flow cytometry. Uptake in confocal images were quantified using Image J analysis program (available from the NIH). As shown in FIG. 19, confocal analysis reveals that Texas Red HA is more rapidly taken up into the progenitor-like tumor cells (defined by CD24−/CD44+ e.g. MDA-MB-231 shown) than into putative non-progenitor cells (defined by CD24+/CD44−, Table 1, e.g. MCF-7 shown). A similar trend was observed when uptake was quantified using flow cytometry (FIG. 23) except that uptake difference was prominent after 30 minutes of HA treatment. While results confirmed our hypothesis for cells grown in culture, they identified the practical range for sorting of subpopulation of highest HA uptake (being at least 1 hour post HA treatment).

Other observation is the distinct initial trend of HA uptake in MCF-7 cells from that of MDA-MB-231 cells which raises the possibility that MCF-7 cells could be of a different progenitor type than MDA-MB-231. This entails further characterization of sub-populations as we proceed.

We next assessed whether these fluorescent hyaluronan polymers can be used to image small nests of MDA-MB-231 cells in vivo using a chick chorioallantoic membrane (CAM) model (See Zijlstra, A., et al., The inhibition of tumor cell intravasation and subsequent metastasis via regulation of in vivo tumor cell motility by the tetraspanin CD151. Cancer Cell, 2008. 13(3): p. 221-34) (FIG. 24). Calcein-green tagged MDA-MB-231 cells were injected into blood vessels in chick embryos grown exvivo. Once calcein-marked tumor cells had extravasated from blood vessels into the surrounding tissue, Cy5.5-HA was injected into blood and uptake into the tumor cells was monitored in real time for 48 hr. However, Cy5.5 HA uptake could not be detected in the extravasted MDA-MB-231 tumor cells. We are currently optimizing the dose and the binding specificity assays in order to establish a protocol for screening new such probes in CAM model comprising the steps of: Fluorescent-HA probes are initially assessed for differential uptake by MDA-MB-231 and MCF-7 breast tumor cell cultures, selected probes are then assessed for uptake into extravasated breast tumor cells in a CAM model and finally into tumor xenografts implanted into mammary fat pads of immune compromised mice.

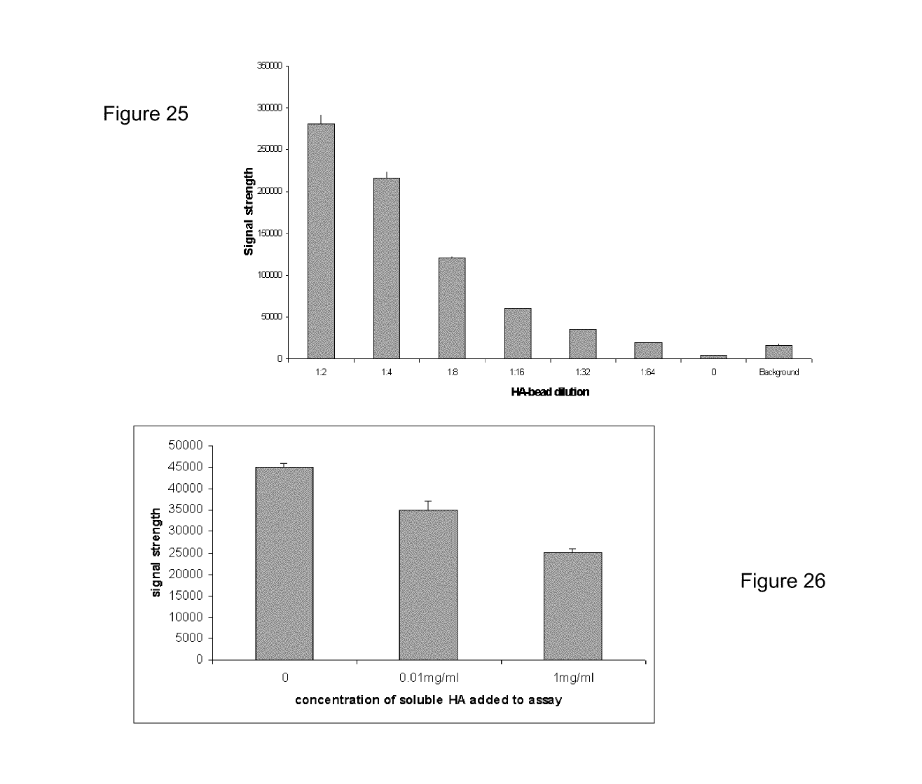

Fluorescent microsphere-HA: We next developed a method for linking HA to fluorescent microspheres (Invitrogen, 200 nm). HA was linked to amine-modified fluorescent microspheres using cyanoborohydride. Beads were tested for their ability to bind to recombinant RHAMM protein, used as an example of an HA binding protein. The specificity of this binding was assessed by quantifying the ability of soluble HA to compete with microsphere-HA for binding to RHAMM. As shown in FIGS. 25 and 26, microsphere-HA exhibit linear binding to recombinant Rhamm and this binding is competed by excess soluble HA indicating that the interaction with Rhamm protein is specific. We have now begun to assess the uptake of microsphere-HA by human breast tumor cell lines using confocal microscopy and flow cytometry. We are also in the process of designing dual mode (MRI/fluorescence) HA nano-particles with either polylactide-co-glyclide as described in Hyung, W., et al., Novel hyaluronic acid (HA) coated drug carriers (HCDCs) for human breast cancer treatment. Biotechnol Bioeng, 2008. 99(2): p. 442-54 and incorporated by reference, or other stealth coatings for use in imaging breast tumor progenitor cells in vivo.

### Example 8

### The Relationship Between HA Uptake and Breast Cancer Cell Line Phenotypes

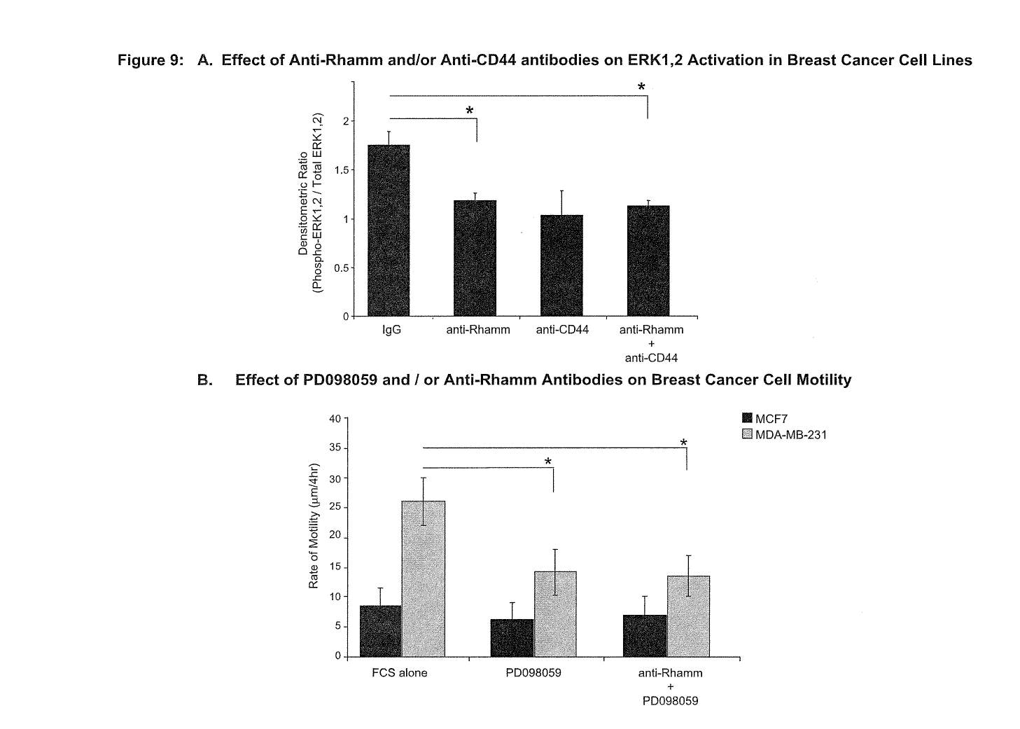

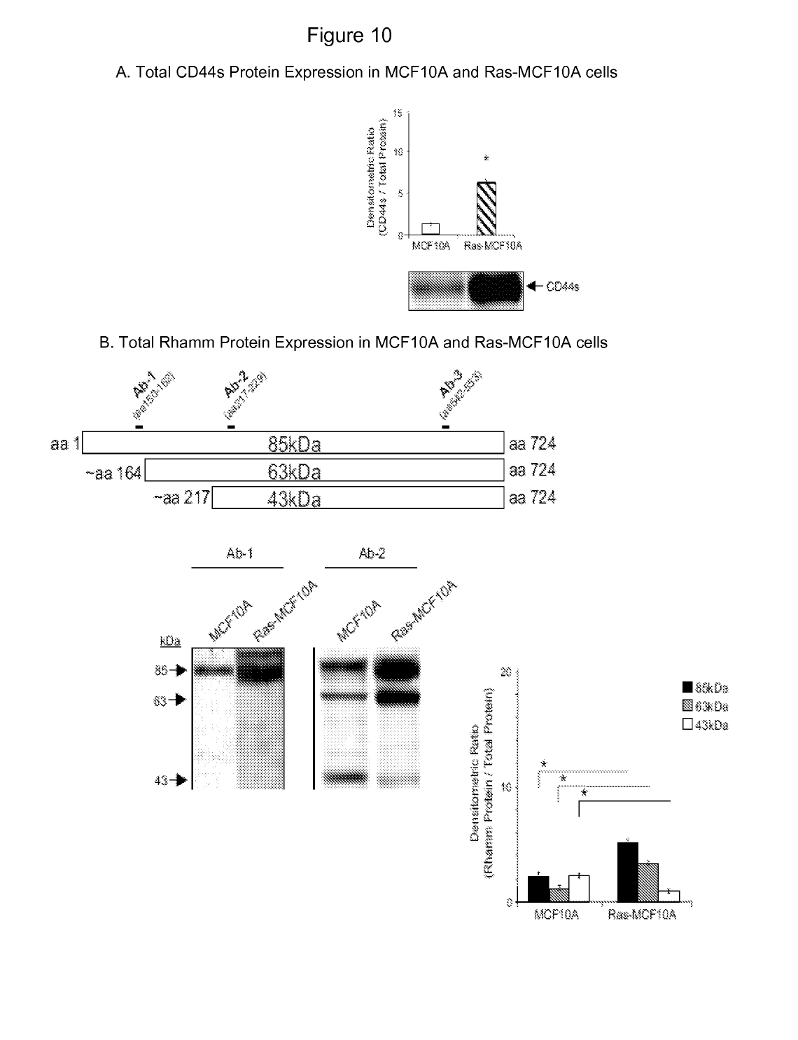

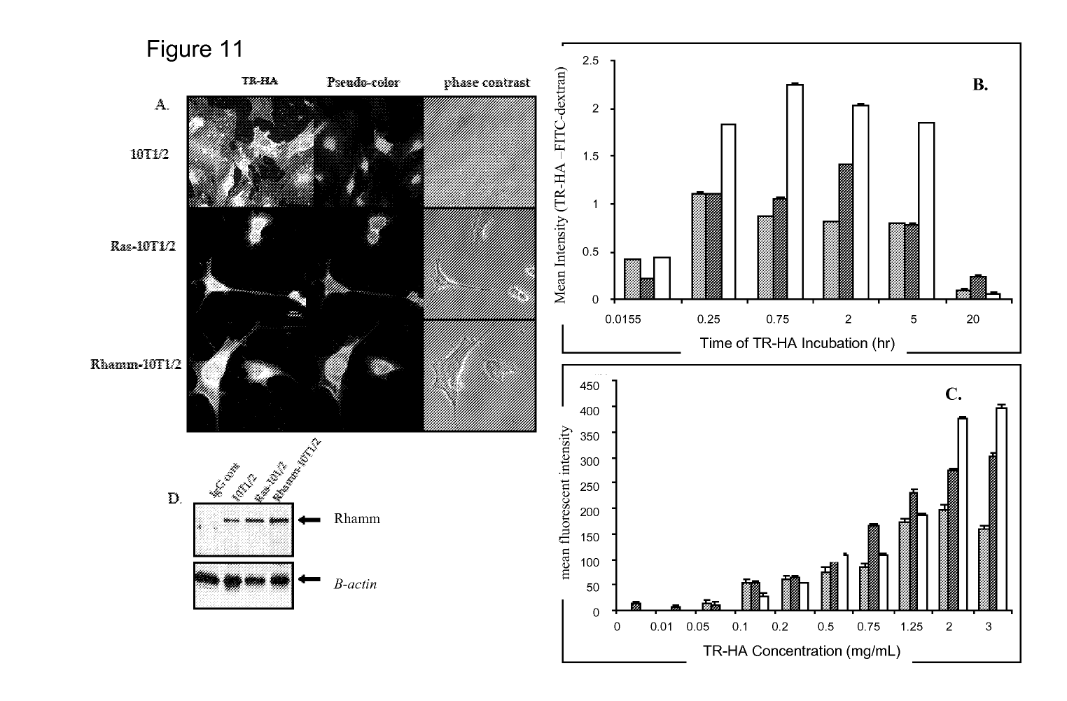

Our preliminary data showed that MDA-MB-231 breast tumor cells (CD44+/CD24−/basal b subtype) endocytosed Texas Red and GD-HA more rapidly and to a greater extent than CD24+/CD44+, CD24+/CD44− and that belonged to the basal b, basal a or luminal breast tumor subtype were selected (Table 1). Four basal B (MDA-MB-231, HS578T, SUM1315MO2 and S1), 1 basal A (HCC-1569) and 3 luminal (MDA-MB-361, MCF-7, and SKBR3) subtypes were chosen to analyze in detail for expression of both CD44 and CD24 using flow cytometry and for HA uptake using confocal microscopy and flow cytometry. Flow cytometry was also used to compare the rates of HA uptake amongst each of these cell lines. Flow cytometry analysis confirmed that MDA-MB-231 and Hs578T breast tumor cell lines are CD24^{-/low}/CD44⁺ and that MDA-MB468, MCF7, and SKBR3 are CD24⁺/CD44^{-/low} (FIG. 27) in agreement with Sheridan, C. et al. ^{[33]}. Using these cell lines, we next assessed if HA uptake is related to CD44 expression levels (FIG. 10),breast tumor subtype or a CD24⁻/CD44⁺ phenotype.

TABLE 1
Characteristics of breast cancer cell lines

| # | Cell Lines | Type | CD44 | CD24 | HA | ER | PR | HER2 | 3D Morphology |
|---|---|---|---|---|---|---|---|---|---|
| 1 | MCF-10A | Basal B | + | | | − | − | +/_WT | |
| 2 | S1 | Basal B | + | − | + | | | | Round |
| 3 | T4-2 | Basal B | + | | | | | | Mass |
| 4 | MDA-MB-231 | Basal B | ++ | − | +++ | − | − | | Stellate |
| 5 | SUM1315MO2 | Basal B | + | + | ++ | − | − | | |
| 6 | HS-578T | Basal B | +++ | − | ++ | − | − | | Stellate |
| 7 | MDA-MB-468 | Basal A | ++ | + | | − | − | | Grape-like |
| 8 | HCC-1569 | Basal A | + | − | + | − | − | + | Mass |
| 9 | BT-20 | Basal A | ++ | | | − | − | | |
| 10 | MCF-7 | Luminal | + | + | ++ | + | + | | Mass |
| 11 | BT474 | Luminal | − | + | | + | + | + | Mass |
| 12 | SK-BR3 | Luminal | − | <+ | + | − | − | + | Grape-like |
| 13 | AU565 | Luminal | | | | − | − | + | Grape-like |
| 14 | MDA-MB-361 | Luminal | − | <+ | + | + | − | + | Grape-like |

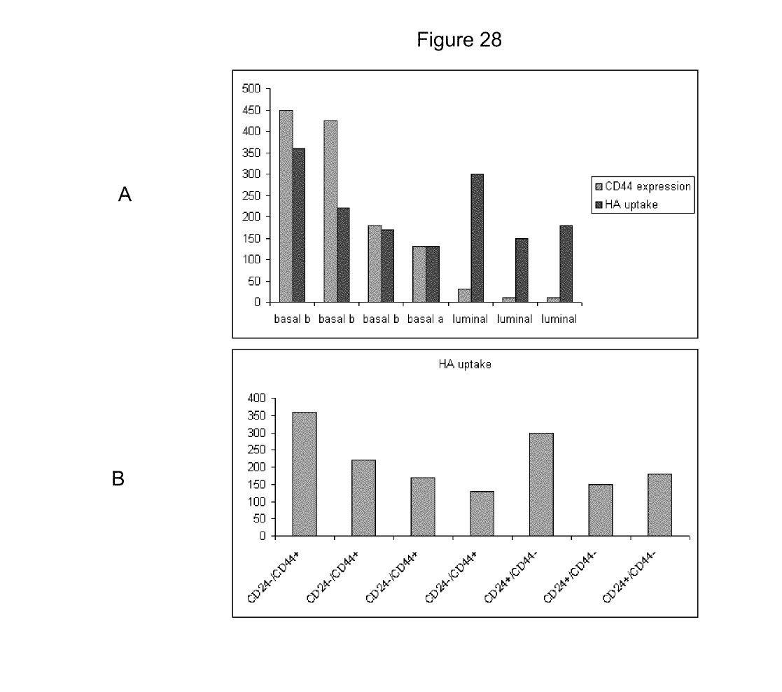

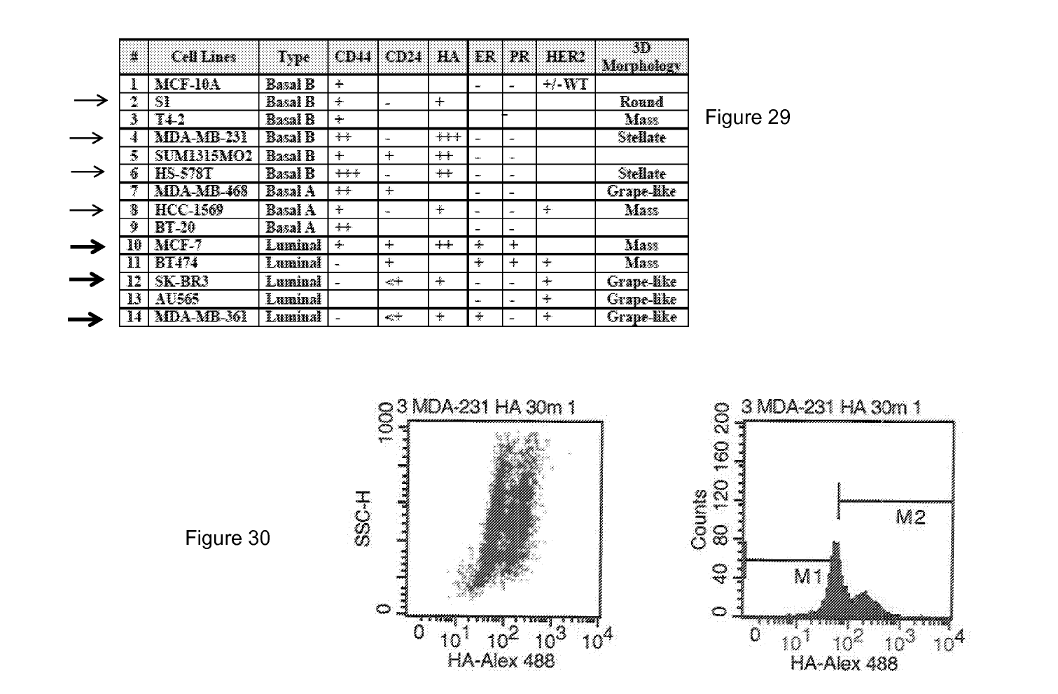

Cell lines expressing higher CD44 levels and a CD24^{-/low}/CD44⁺ phenotype grouped in the basal B tumor subtype while CD24⁺/CD44^{-/low} expressing tumor cells grouped within the luminal tumor subtype (FIG. 28). However, HA uptake did not relate to CD44 expression, or CD24⁻/CD44⁺ phenotype (FIG. 28). Uptake was however generally greater in molecular subtypes basal A and B. Since quantification of HA uptake was done at a single timepoint (1 hour), we also considered the possibility that rates of uptake differ in a complex manner with CD24−/CD44+ phenotype, which would not be detected in a single time point. Analysis of HA uptake overtime revealed a difference in initial rates of HA uptake between MDA-MB-231 and MCF-7 breast tumor cells, which was identical to previous preliminary data (Collis, L. Masters Thesis, University of Toronto, 1999), but this differential timing in uptake was not observed amongst any of the other breast tumor cell lines (FIG. 29). Since these breast cancer cell lines are composed of phenotypic subpopulations that differ in the extent to which they express for example, CD44, our assessment of the shift of signal for total population rather than subpopulations may have prevented detection of a relationship between CD44 and HA uptake. An alternative possibility is that other HA receptors, such as LYVE1, layillin and RHAMM/HMMR are required for HA uptake. To address both of these issues, we analyzed the heterogeneity of HA uptake using flow cytometry and we have begun to quantify expression of additional HA receptors beginning with RHAMM/HMMR since it is a prognostic factor in poor outcome of breast cancer patients (Pujana, M. A., et al., Network modeling links breast cancer susceptibility and centrosome dysfunction. Nat Genet, 2007. 39(11): p. 1338-49; Wang, C., et al., The overexpression of RHAMM, a hyaluronan-binding protein that regulates ras signaling, correlates with overexpression of mitogen-activated protein kinase and is a significant parameter in breast cancer progression. Clin Cancer Res, 1998. 4(3): p. 567-76) and a novel breast tumor susceptibility gene (Pujana, M. A., et al., Network modeling links breast cancer susceptibility and centrosome dysfunction. Nat Genet, 2007. 39(11): p. 1338-49). As shown in FIG. 27, flow cytometry analysis of uptake of Alexa fluor 488-HA by MDA-MB-231 breast tumor cells reveals distinct subpopulations that display uptake differences.

We have developed a method for sorting subpopulations based upon Alexa 488 HA uptake and demonstrated that these cells are viable after sorting. We are in the process of characterizing their surface phenotype (e.g. CD24−/CD44+) and assessing their cell cycle status.

Experiments were also initiated to assess the importance of CD44 and RHAMM/HMMR in HA uptake in transformed vs. non transformed cells and to assess the role of these HA receptors in determining aggressive behavior of human breast cancer cell lines representing Basal B and luminal subtypes. Results from these experiments are expected to clarify if HA uptake/HA receptor expression is related to tumor cell aggression even if it is not associated with a CD24−/CD44+ tumor progenitor phenotype.

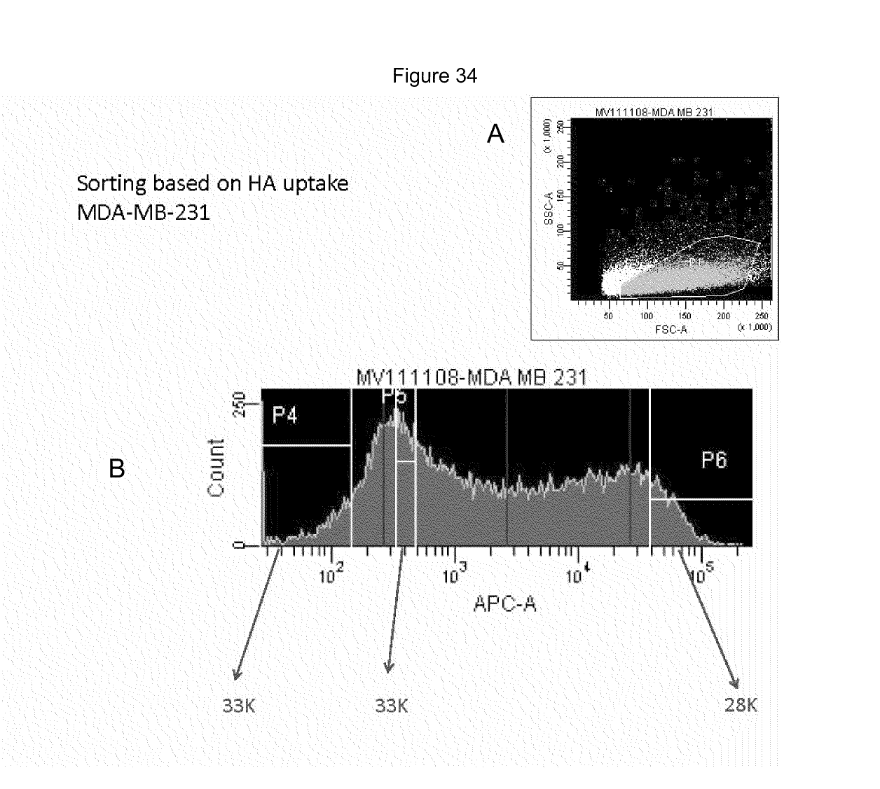

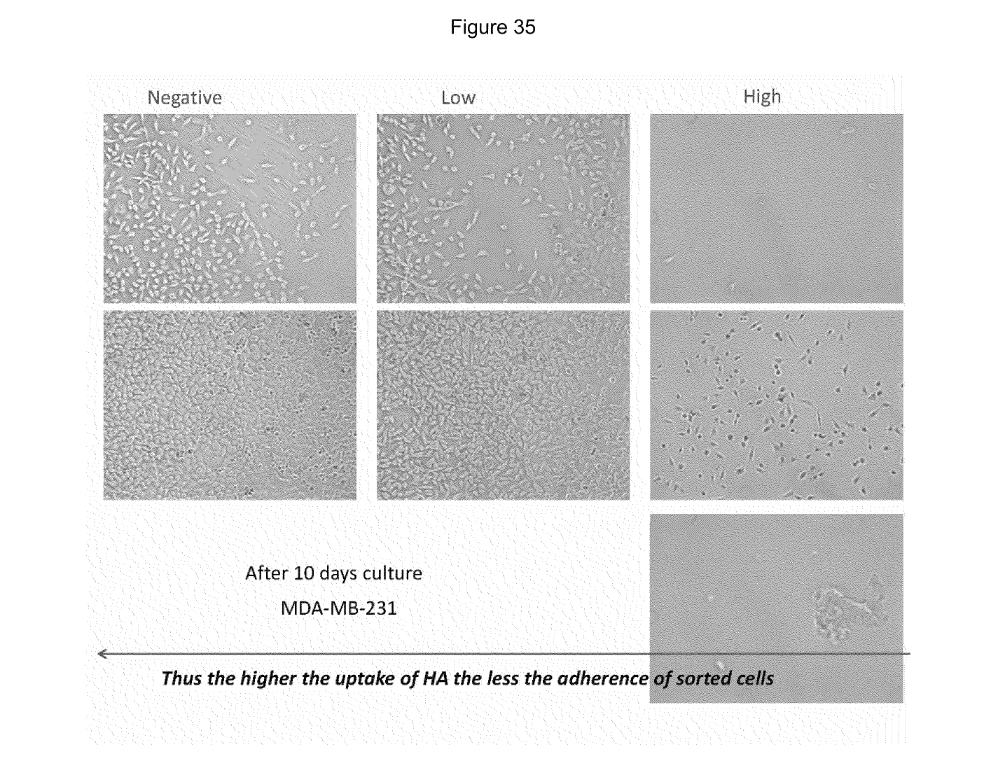

Referring now to FIG. 35, FACS analysis showed that these progenitor like breast cancer cells have subpopulations that taken up very little HA (eg. Culture figures on the left) and very high levels of HA (culture figures on the right hand side of FIG. 35). Thus FIG. 34 shows the uptake of fluorescent HA varies form 100 to >100,000 units (x axis scale on bottom of graph). When cells that take up very little HA are cultured and compared to cells that take up a lot of HA (FIG. 35), the cell morphology observed of high uptake cells is very variable consistent with them being plastic which is consistent with these cells having more of a progenitor phenotype than low uptake cells

In summary, when a number of human breast cancer cell lines were analyzed for HA uptake (FIG. 29), it became clear that the cells taking the highest amount of HA were of a basal (a and b, light gray arrows) molecular subtype while luminal subtypes generally took up less HA (darker arrows).

### Example 9

### Roles of CD44 and Rhamm and HA Uptake in Transformed Cells

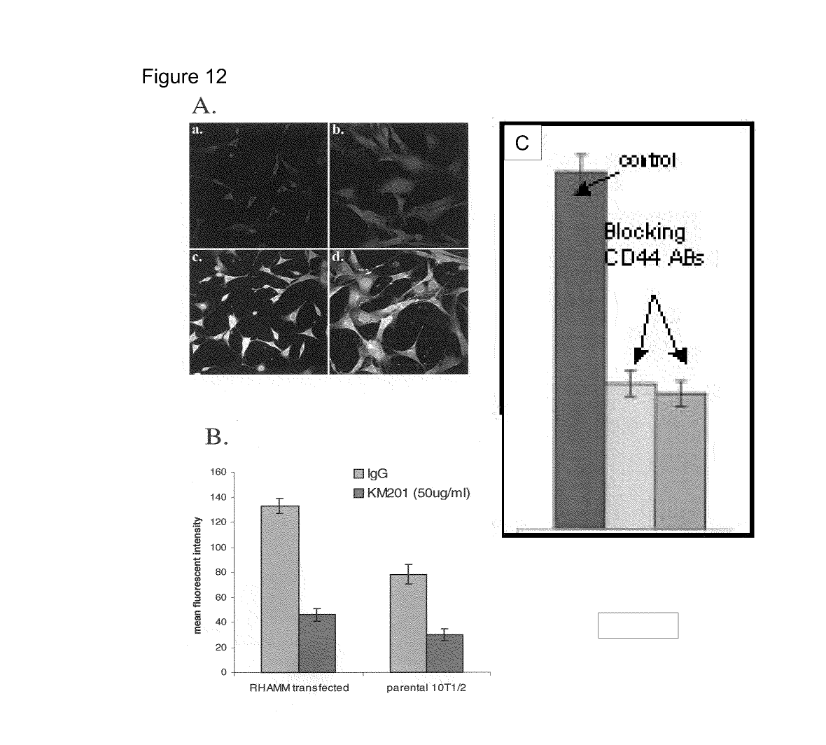

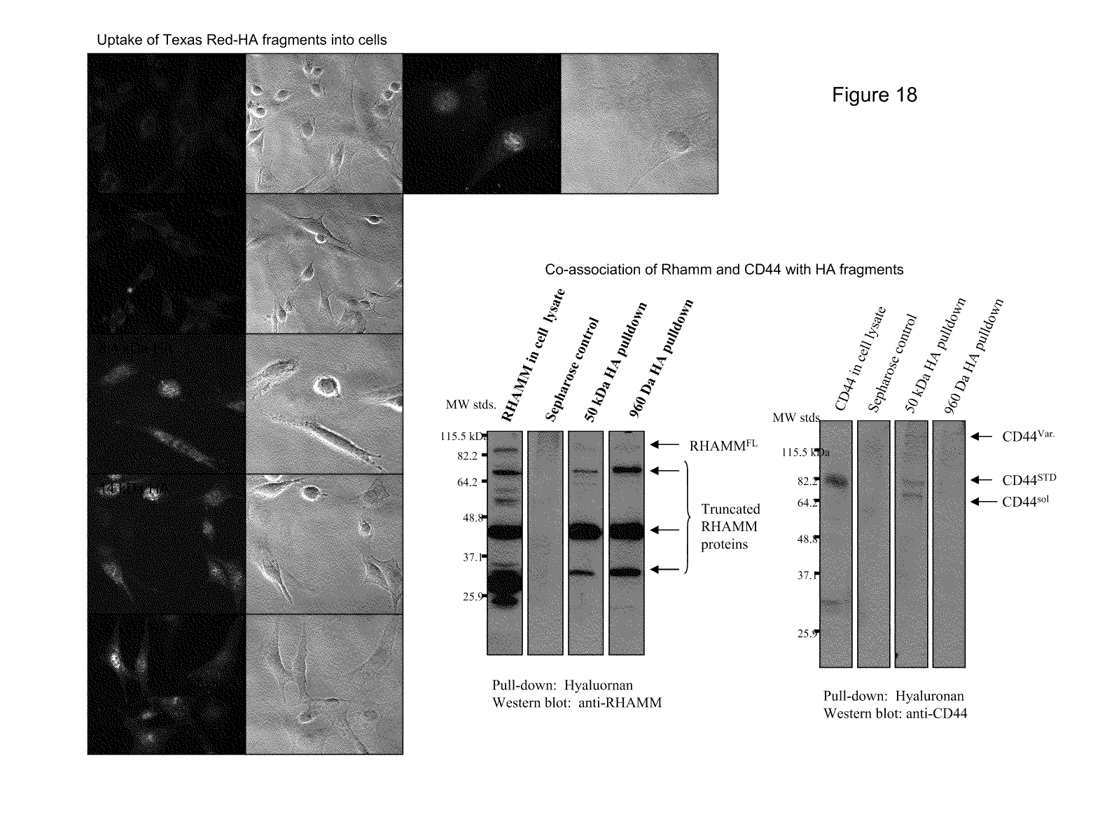

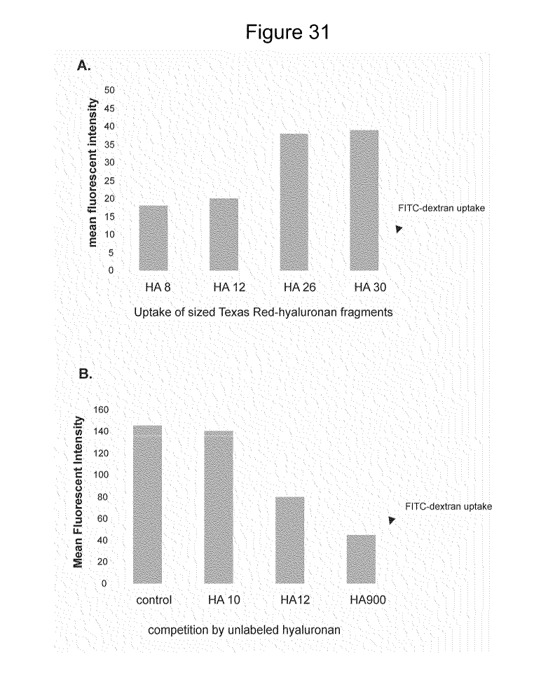

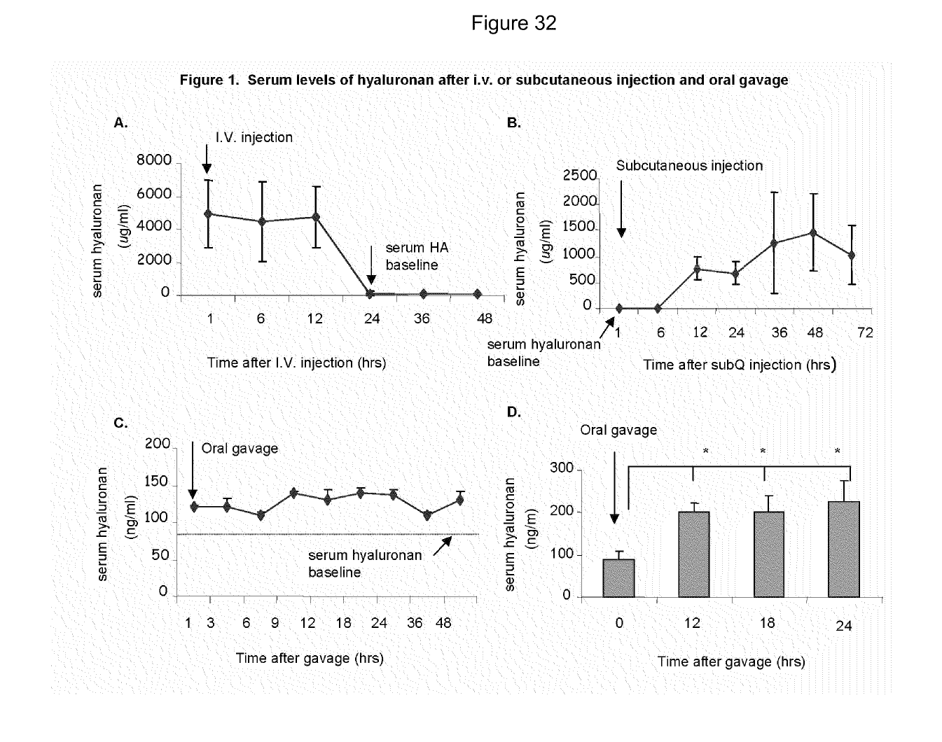

In order to assess the relative importance of CD44 and RHAMM on the rapidity of HA uptake, we utilized mouse fibroblasts since we had developed immortalized fibroblast lasts that are Rhamm^{-/-}, CD44^{-/-} and Rhamm^{-/-}:CD44^{-/-} originally isolated from Rhamm−/−, CD44−/− and double knockout mouse strains that we developed. Use of these cell lines has permitted us to unequivocally assess not only the relative importance of Rhamm vs CD44 in HA uptake but to determine if other HA receptors can compensate for the loss of Rhamm and CD44 in this function. Uptake of Texas Red HA was found to be saturable (data not shown), and HA is size dependent and is competed for by soluble unlabeled HA (FIGS. 31A and B) indicating that it is receptor mediated. Rapid uptake of Texas-Red HA occurs immediately following subculture of cells (e.g. in the first 2-12 hrs, see FIG. 24), and after scratch wounding and in Ras-transformed fibroblasts (data not shown). These results indicate that HA uptake or metabolism is greatest during response-to-injury (e.g. subculture response to trypsin and to scratch wounding) and after neoplastic transformation. These results are consistent with evidence that HA metabolism is enhanced in some human cancers (Alvarez, A. and V. B. Lokeshwar, Bladder cancer biomarkers: current developments and future implementation. Curr Opin Urol, 2007. 17(5): p. 341-6). Rapid HA uptake correlates with expression of Rhamm and CD44 cell surface display and both anti-CD44 antibodies and Rhamm peptide antagonists block uptake (FIG. 12). Cy5.5-HA uptake is highest in mouse embryonic fibroblasts (MEF) that express both Rhamm and CD44 than in either Rhamm^{-/-} or CD44^{-/-} MEF (data not shown). These results suggest that both CD44 and Rhamm are required for rapid uptake of HA into transformed cells. We reasoned that if CD44 and Rhamm are both necessary for rapid uptake of HA, surface forms of both proteins should bind to HA. To assess this, we linked HA to Sepharose as we have previously described in Turley, E. A., Purification of a hyaluronate-binding protein fraction that modifies cell social behavior. Biochem Biophys Res Commun, 1982. 108(3): p. 1016-24, hereby incorporated by reference, and used this reagent in a pull-down assay using lysates from mouse fibroblasts that express both Rhamm and CD44 (FIG. 18). As expected, native HA of similar MW that we have used in the above described experiments (e.g. 220 kDa) binds to both Rhamm and standard and variant CD44 forms. However, small HA fragments bind only to Rhamm. These results suggest that if HA is highly fragmented binding to CD44 will be restricted. Rhamm is a peripheral protein and binds to integral cell surface proteins such as CD44. These results predict that Rhamm may associate with other HA receptors (e.g. Toll Like Receptors2, 4 (Land, W., Innate alloimmunity: history and current knowledge. Exp Clin Transplant, 2007. 5(1): p. 575-84; Jiang, D., J. Liang, and P. W. Noble, Hyaluronan in tissue injury and repair. Annu Rev Cell Dev Biol, 2007. 23: p. 435-61), Layilin (Chen, Z., et al., Down-regulation of Layilin, a novel hyaluronan receptor, via RNA interference inhibits invasion and lymphatic metastasis of A549 cells. Biotechnol Appl Biochem, 2007) or other as yet unidentified HA receptors to promote uptake of small fragments. These results provide additional rationale for assessing the surface display of several HA receptors in addition to CD44.

### Example 10

### Role of CD44 and RHAMM in Aggression of Human Breast Cancer Cells

Although CD44 expression is linked to a progenitor breast tumor and a basal phenotype, it is not clearly associated with rapid HA uptake. To assess if CD44/RHAMM/HA interactions (e.g. FIG. 18) that are associated with rapid uptake of HA in fibroblasts are linked to functional characteristics associated with aggression in breast cancer cell lines, we compared the association of these HA receptors in 3 basal B breast cancer cell lines and a luminal breast cancer cell line. See Table 1 above. Co-association of CD44 and RHAMM are greatest in breast cancer cell lines with an active Ras-Erk pathway, regardless of the subtype. Thus, the association is most apparent in MDA-MB-231 (basal b, CD24−/CD44+) and Ras-MCF10A (basal b) tumor cells and is less apparent in MCF (luminal, CD24+/CD44−) and MCF10A (basal b). Furthermore, tumor cells exhibiting a strong degree of CD44/RHAMM interaction respond to HA or serum by increased cell motility/invasion and this effect is blocked by inhibitors of Erk1,2 activation. Although mutations of the Ras-Erk pathway are rare in breast cancer, hyper-activation of this pathway is common and has been linked to poor prognosis. These results confirm that CD44/RHAMM interactions are associated with tumor aggression, particularly when Map kinase signaling pathways are activated, and provide evidence to support further assessment of the relationship amongst RHAMM expression, HA uptake, progenitor phenotype and tumor subtype.

### Example 11

### Kinetic Analysis of HA Injected I.V. into Humans and into Rodents

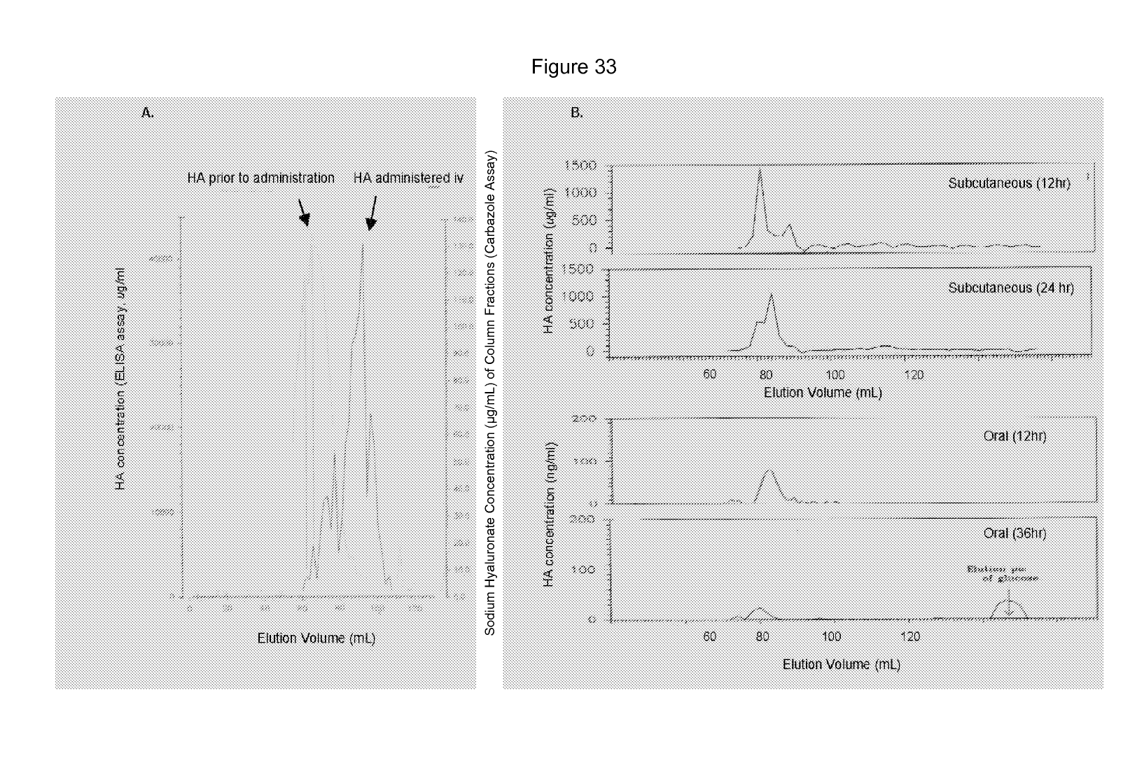

To assess if tagged-HA imaging agents exhibit a sufficiently long half life following their injection I.V., we quantified the kinetics of HA elimination from plasma by analyzing data obtained from Hyal Corporation (Toronto, Calif.) using healthy human subjects and rats. In both humans and rats, HA injected I.V. (1-12 mg/kg) was well tolerated and analysis of HA serum levels revealed a T1/2 of 12 hrs (for a 1.5 mg/kg dose) (FIGS. 32 and 33). These results indicate that the T1/2 of tagged-HA nanoparticles is sufficiently long for use as imaging agents in both experimental models (e.g. rodents) and humans.

Table 2 below is a summary of the kinetic analysis of HA in serum by different routes of administration.

TABLE 2
Pharmacokinetic parameters of HA in serum

| | Cmax | Tmax | | |
|---|---|---|---|---|
| Route of administration | (ug/ml) | (hr) | AUC@72 h¹ | F² |
| Intravenous | 5096.00 | 12 hr | 0 | 1.00 |
| Subcutaneous | 1454.00 | 72 hr | 72 | .79 |
| Oral | 0.15 | 36 hr | 6.01 | .0001 |

Serum hyaluronan levels were measured using a competitive binding ELISA. Assays were done in triplicate and background serum levels of hyaluronan were subtracted from experimental levels. Values used for kinetic analyses are from one experimental series.
Cmax = maximum concentration achieved
Tmax = time after administration when maximum concentration was achieved
¹AUC = area under the curve
²absorption rate constant

Thus, it can be seen that intravenous and subcutaneous injection may provide useful routes of administration for in vivo imaging of cancer progenitor cell populations.

The above examples are provided to illustrate the invention but not to limit its scope. Other variants of the invention will be readily apparent to one of ordinary skill in the art and are encompassed by the appended claims. All publications, databases, and patents cited herein are hereby incorporated by reference for all purposes.

What is claimed is:

1. A method of identifying cancer progenitor cells, the method comprising:
providing a sample; and
detecting a presence of CD44/Rhamm complexes,
wherein the presence of CD44/Rhamm complexes indicates the sample contains a cancer progenitor cell.

2. The method of claim 1, wherein the sample is taken from a mammal.

3. The method of claim 2, wherein the mammal is a human.

4. The method of claim 1, wherein the sample is a biopsy taken from the group consisting of breast cancer, colon cancer, gastric cancer, glioma, parenchymal tumors, leukemias, multiple myeloma, immune-cell related tumors, desmoid tumors, mesenchymal related tumors, skin cancers, basal cell carcinoma, and melanoma.

5. The method of claim 1, wherein the presence of CD44/Rhamm complexes is detected by contacting the cancer cell with an antibody or ligand that specifically binds to the CD44/Rhamm complex.

6. The method of claim 5, wherein the antibody or ligand is labeled with a detectable label.

7. The method of claim 1, wherein the presence of CD44/Rhamm complexes is detected by contacting the cancer cell with a first antibody or small chemical or peptide mimetic or ligand that specifically binds to CD44 and a second antibody, small chemical or peptide mimetic or ligand that specifically binds to Rhamm.

8. A method of prognosing cancer, the method comprising:
providing a cancer cell; and
detecting a presence of CD44/Rhamm complexes,
whereby the presence of the CD44/Rhamm complexes indicates that the cancer cell is aggressively metastatic.

9. The method of claim 8, wherein the cancer cell is in a mammal.

10. The method of claim 9, wherein the mammal is a human.

11. The method of claim 8, wherein the cancer is breast cancer, colon cancer, gastric cancer, glioma, parenchymal tumors, leukemias, multiple myeloma, immune-cell related tumors, desmoid tumors, mesenchymal related tumors, skin cancers, basal cell carcinoma, and melanoma.

12. The method of claim 8, wherein the presence of CD44/Rhamm complexes is detected by contacting the cancer cell with an antibody, CD44 or Rhamm small chemical or peptide mimetic, or a ligand, small chemical or peptide mimetic that specifically binds to the CD44/Rhamm complex.

13. The method of claim 12, wherein the antibody is labeled with a detectable label.

14. The method of claim 8, wherein the presence of CD44/Rhamm complexes is detected by contacting the cancer cell with a first antibody, small chemical or peptide mimetic that specifically binds to CD44 and a second antibody, small chemical or peptide mimetic that specifically binds to Rhamm.

15. A method of inhibiting cancer cell metastasis, the method comprising:
contacting a cancer cell with a compound that inhibits formation of CD44/Rhamm complex.

16. The method of claim 15 wherein the compound is an antibody, a small molecule, a mimetic, a peptide, a siRNA, an antisense oligonucleotide, or an aptamer.

17. The method of claim 15 wherein the compound is an antibody that specifically binds CD44.

18. The method of claim 15 wherein the compound is an antibody that specifically binds Rhamm.

19. The method of claim 15 where in the cancer cell is a breast cancer cell.

20. The method of claim 15 wherein the cancer cell is in a mammal.

21. The method of claim 20 wherein the mammal is a human.

22. A method of identifying a compound that inhibits cancer cell metastasis, the method comprising:
contacting a cancer cell with a compound suspected of inhibiting metastasis of cancer cells;
detecting CD44/Rhamm complexes;
whereby a reduction in the amount of CD44/Rhamm complexes identifies the compound as an inhibitor of cancer cell metastasis.

23. The method of claim 22, wherein the compound is selected from the group consisting of an antibody, a small molecule, a mimetic, a peptide, a siRNA, an antisense oligonucleotide, or an aptamer.

24. The method of claim 23, wherein the cancer cell is in a mammal.

25. The method of claim 24, wherein the mammal is a rodent.

26. A probe for identifying tumorigenic cell populations, comprising a targeting component and an imaging component, wherein said targeting component specifically binds to the CD44/Rhamm complexes, and wherein said imaging component is a detectable label.

27. The method of claim 5, wherein the ligand is hyaluronan, a hyaluronan fragment, hyaluronan mimetic peptides or small chemical mimics.

28. The method of claim 7, wherein the ligand is hyaluronan, a hyaluronan fragment, hyaluronan mimetic peptides or small chemical mimics
